(19) United States
(12) Patent Application Publication
(10) Pub. No.: US 2026/0294462 A1
(43) Pub. Date: Oct. 1, 2026

(54) ROTATION CONTROL ASSEMBLIES FOR TUBES

(71) Applicant: Edwards Lifesciences Corporation, Irvine, CA (US)

(72) Inventor: Peleg Harel, Haifa (IL)

(73) Assignee: Edwards Lifesciences Corporation, Irvine, CA (US)

(21) Appl. No.: 19/683,521

(22) Filed: May 20, 2026

Related U.S. Application Data
(63) Continuation of application No. PCT/US2024/056131, filed on Nov. 15, 2024.
(60) Provisional application No. 63/602,267, filed on Nov. 22, 2023.

(51) Int. Cl. A61B 17/32; A61F 2/24; A61M 25/01; A61M 25/00

(52) U.S. Cl. CPC A61B 17/32002; A61F 2/2427; A61M 25/0136; A61F 2220/0016; A61F 2230/0091; A61M 2025/0095

(57) ABSTRACT

A delivery system includes a rotation control assembly having a rotatable body defining an aperture, and a thin-wall tube extending through the aperture and comprising a first window and a second window. The rotation control assembly further includes a first protrusion and a second radial protrusion extending from the rotatable body into the first window and the second window. The first and second protrusions define first and second contact surfaces that face circumferentially opposite directions, such that rotation of the rotatable body in a clockwise or counterclockwise direction causes the appropriate contact surface to push against a contact edge of the corresponding window, to impart rotational movement of the tube therewith.

### CROSS-REFERENCE TO RELATED APPLICATIONS

This application is a continuation of International Application No. PCT/US2024/056131, filed Nov 15, 2024, which claims the benefit of U.S. Provisional Application No. 63/602,267, filed Nov 22, 2023, which is incorporated by reference herein.

### FIELD

The present disclosure relates to systems and assemblies configured to facilitate rotational movement of tubes, such as thin-walled tubes, and to methods and devices for angularly orienting components of systems designed to form an opening in a target tissue, such as a leaflet of a valve in which a guest prosthetic valve can be optionally implanted.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches, such as transcatheter aortic valve replacement (TAVR), are used in various procedures to deliver prosthetic medical devices to locations inside the body that are not readily accessible by surgery or where access without surgery is desirable.

Transcatheter aortic valve replacement (TAVR) is one example of a minimally-invasive surgical procedure used to replace a native aortic valve. In one specific example of the procedure, an expandable prosthetic heart valve is mounted in a crimped state on the distal end of a delivery apparatus and advanced through the patient’s vasculature (for example, through a femoral artery and the aorta) to the heart. The prosthetic heart valve is positioned within the native valve and expanded to its functional size.

A variant of TAVR is valve-in-valve (ViV) TAVR, where a new prosthetic heart valve replaces a previously implanted prosthetic valve. In one specific example of the procedure, a new expandable prosthetic heart valve ("guest valve") is delivered to the heart in a crimped state, as described above for the "native" TAVR. The guest valve is positioned within the previously implanted prosthetic valve ("host valve") and then expanded to its functional size. The host valve in a ViV TAVR procedure can be a surgically implanted prosthetic valve or a transcatheter prosthetic valve. The term "host valve" is also used herein to refer to the native aortic valve in a native TAVR procedure.

### SUMMARY

One potential technique for mitigating the risk of coronary ostial obstruction involves formation of a hole in one or more leaflets of the host valve (which can be an aortic bioprosthetic valve or a native aortic valve). A guest prosthetic valve can be optionally placed within the leaflet hole and expanded in a manner that tears the host leaflet and prevents it from obstructing the coronary ostium. Rotational movement of one or more tubes of a system that includes an appropriate cutting or lacerating apparatus may be required, such as rotation of an anchor designed to grasp and stabilize a leaflet during penetration of a lacerating member, such as a needle, therethrough, and/or angularly orienting an optionally bent lacerating member towards the leaflet in vicinity of the coronary ostium. However, the ability to rotate such a tubular component by a mechanism in the handle of the system may be challenging due to the small dimensions of the tube.

According to some aspects of the disclosure, there is provided a system comprising a handle comprising a rotation control assembly, the rotation control assembly comprising a rotatable body defining an aperture.

In some examples, the rotation control assembly comprises at least one first protrusion comprising a first contact surface and configured to extend radially into the aperture.

In some examples, the rotation control assembly comprises at least one second protrusion comprising a second contact surface and configured to extend radially into the aperture.

In some examples, the rotation control assembly comprises a tube comprising a tube wall having a tube wall thickness and defining a tube lumen, wherein the tube extends through the aperture.

In some examples, the tube comprises at least one first window radially extending through the tube wall and defining a first contact edge.

In some examples, the tube comprises at least one second window radially extending through the tube wall and defining a second contact edge.

In some examples, the at least one first protrusion extends into the at least one first window, and the at least one second protrusion extends into the at least one second window.

In some examples, the first contact surface and the second contact surface face circumferentially opposite directions.

In some examples, when the rotatable body is rotated in a first circumferential direction, the first contact surface is pushed against the first contact edge, causing the tube to rotate in the first circumferential direction along with the rotatable body.

In some examples, when the rotatable body is rotated in a second circumferential direction which is opposite to the first circumferential direction, the second contact surface is pushed against the second contact edge, causing the tube to rotate in the second circumferential direction along with the rotatable body.

In some examples, the at least one first protrusion and the at least one second protrusion do not protrude into the tube lumen.

In some examples, the tube wall thickness is not greater to 200µ.

In some examples, the tube wall thickness is between 50µ and 200µ.

In some examples, the tube wall thickness is less than 15% of an outer diameter of the tube.

In some examples, the tube wall thickness is less than 10% of the outer diameter of the tube.

In some examples, the tube wall thickness is less than 10% of the diameter of the aperture.

In some examples, a maximal contact surface radial length defined by any of the first contact surface and the second contact surface is configured to prevent extension of any of the at least one first protrusion and any of the at least one second protrusion into the tube lumen.

In some examples, the at least one first protrusion comprises two first protrusions, and the at least one second protrusion comprises two second protrusions.

In some examples, the first contact surface of each of the two first protrusions is circumferentially aligned with the second contact surface of the corresponding second radial protrusion of the two second protrusions.

In some examples, the at least one first protrusion comprises one first protrusion, and the at least one second protrusion comprises one second protrusion.

In some examples, the one first protrusion is axially offset from the one second protrusion.

In some examples, the first contact surface of the one first protrusion is circumferentially aligned with the second contact surface of the one second protrusion.

In some examples, the one first protrusion is circumferentially offset from the one second protrusion.

In some examples, the one first protrusion is axially aligned with the one second protrusion.

According to some aspects of the disclosure, there is provided a method comprising advancing a system comprising a tube, over a guidewire, to a target tissue, the system comprising a handle comprising a rotation control assembly.

In some examples, the rotation control assembly comprises a rotatable body defining an aperture through which the tube extends.

In some examples, the rotation control assembly comprises a first protrusion extending radially from the rotatable body into a first window of the tube.

In some examples, the rotation control assembly comprises a second protrusion extending radially from the rotatable body into a second window of the tube.

In some examples, the method comprises rotating the rotatable body in a first circumferential direction or a second circumferential direction.

In some examples, rotating the rotatable body in the first circumferential direction causes a first contact surface thereof to push against a first contact edge of the first window, thereby causing the tube to rotate in the first circumferential direction along with the rotatable body.

In some examples, rotating the rotatable body in the second circumferential direction causes a second contact surface thereof to push against a second contact edge of the second window, thereby causing the tube to rotate in the second circumferential direction along with the rotatable body.

In some examples, the first contact surface and the second contact surface face circumferentially opposite directions.

In some examples, the method further comprises forming, with a perforating member tip of a perforating member of the system extending distally from the handle, a pilot puncture within a target tissue.

In some examples, the perforating member comprises a needle, wherein the perforating member lumen comprises a needle lumen, and wherein the perforating member tip comprises a needle tip.

In some examples, the method further comprises, prior to the forming the pilot puncture, transitioning the needle to an uncovered bent state thereof.

In some examples, the needle is the tube.

In some examples, the rotating the rotatable body comprises orienting the needle tip, in the uncovered bent state of the needle, towards the target tissue.

In some examples, the system further comprises a steerable tube assembly defining a steerable assembly lumen through which the perforating member extends.

In some examples, the steerable tube assembly comprises an inner bendable tube.

In some examples, the steerable tube assembly comprises an outer bendable tube disposed around the inner bendable tube.

In some examples, the steerable tube assembly comprises a pull-member.

In some examples, the pull-member comprises a pull-ring portion affixed to an inner tube distal end portion of the inner bendable tube and to an outer tube distal end portion of the outer bendable tube.

In some examples, the pull-member comprises an elongated pull-arm extending proximally from the pull-ring portion and which is slidingly movable between and relative to the inner bendable tube and the outer bendable tube.

In some examples, the method further comprises, prior to the forming the pilot puncture, bending a distal portion of the steerable tube assembly by proximally pulling the elongated pull-arm.

In some examples, the tube comprises at least one of the outer bendable tube and the inner bendable tube.

In some examples, the rotating the rotatable body comprises orienting a distal tip portion of the steerable tube assembly towards the target tissue.

In some examples, the system further comprises a helical anchor at a distal end of an anchor shaft, the anchor shaft defining an anchor shaft lumen through which the perforation member extends.

In some examples, the anchor shaft is the tube.

In some examples, the rotating the rotatable body causes the anchor shaft and the helical anchor to rotate therewith.

In some examples, the rotating the rotatable body comprises, prior to the forming the pilot puncture, securing the helical anchor to the target tissue.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

### In the Figures

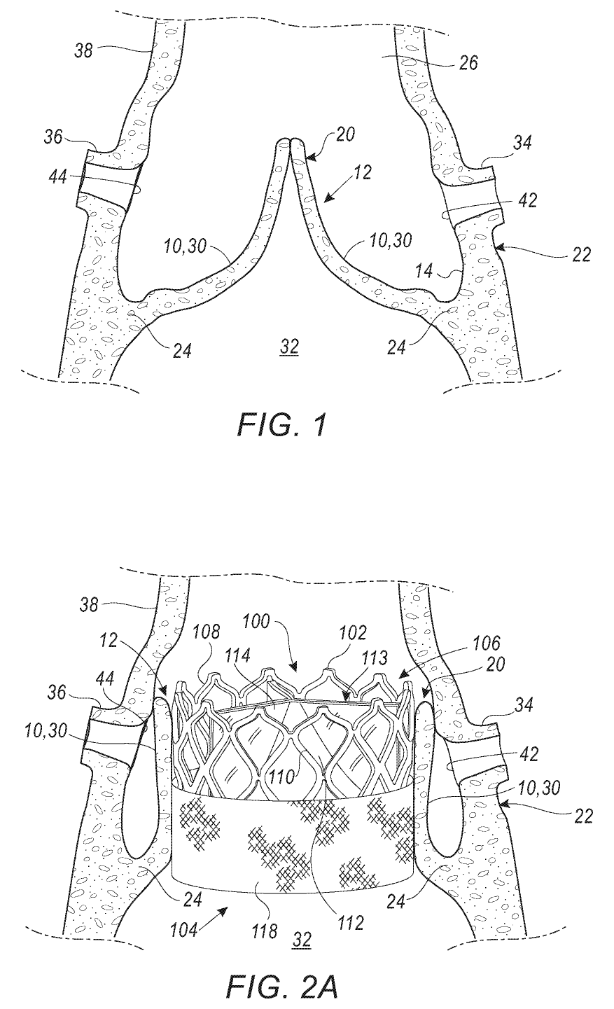

FIG. 1 is a sectional view of an aortic root.

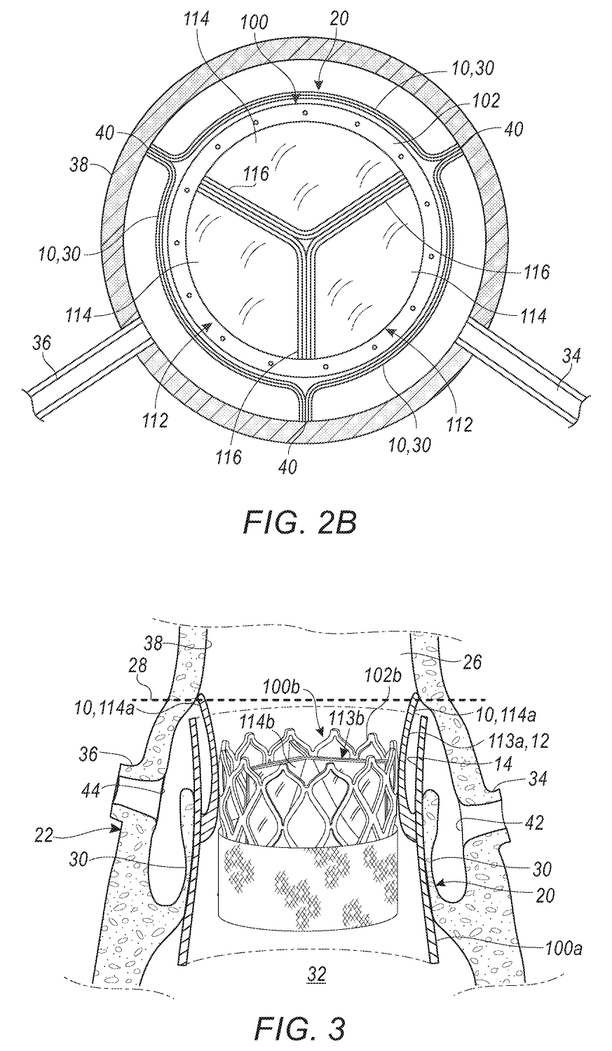

FIG. 2A shows a cross-sectional view of a prosthetic heart valve implanted in the native aortic valve of within the aortic root of FIG. 1, according to an example.

FIG. 2B shows the implanted prosthetic heart valve of FIG. 1A as viewed from the ascending aorta, according to an example.

FIG. 3 shows a valve-in-valve implantation within the native aortic valve of FIG. 1, according to an example.

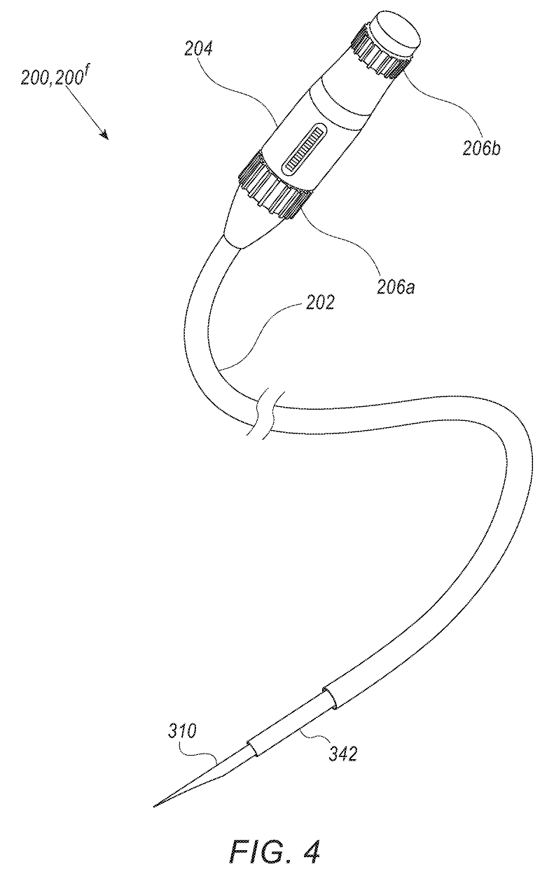

FIG. 4 shows an exemplary system comprising a thin-walled tube extending distally from a handle.

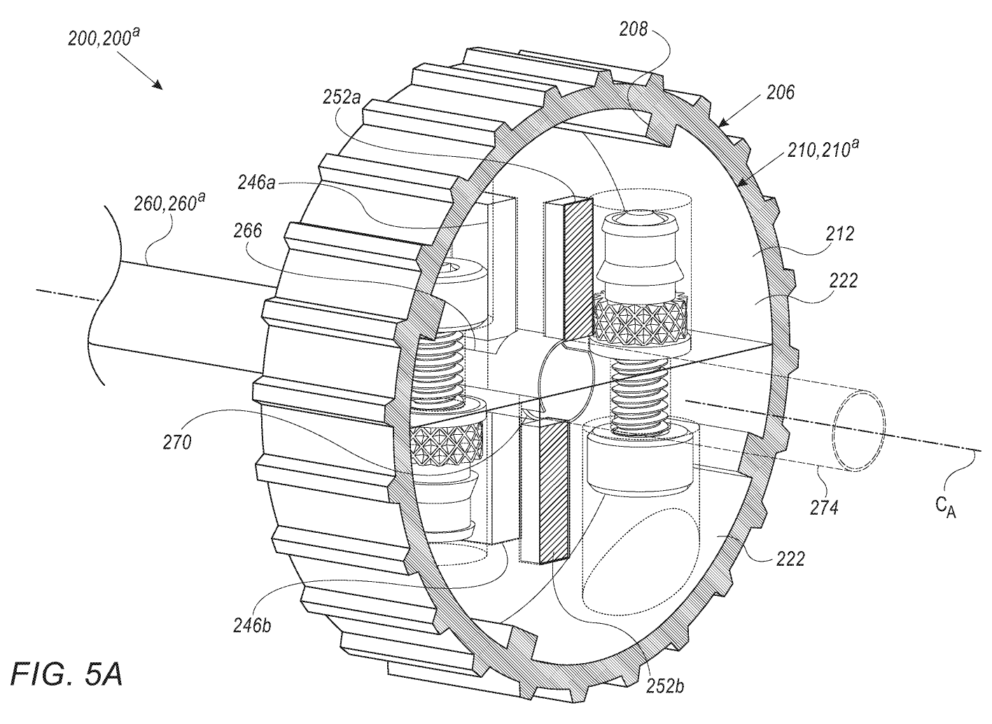

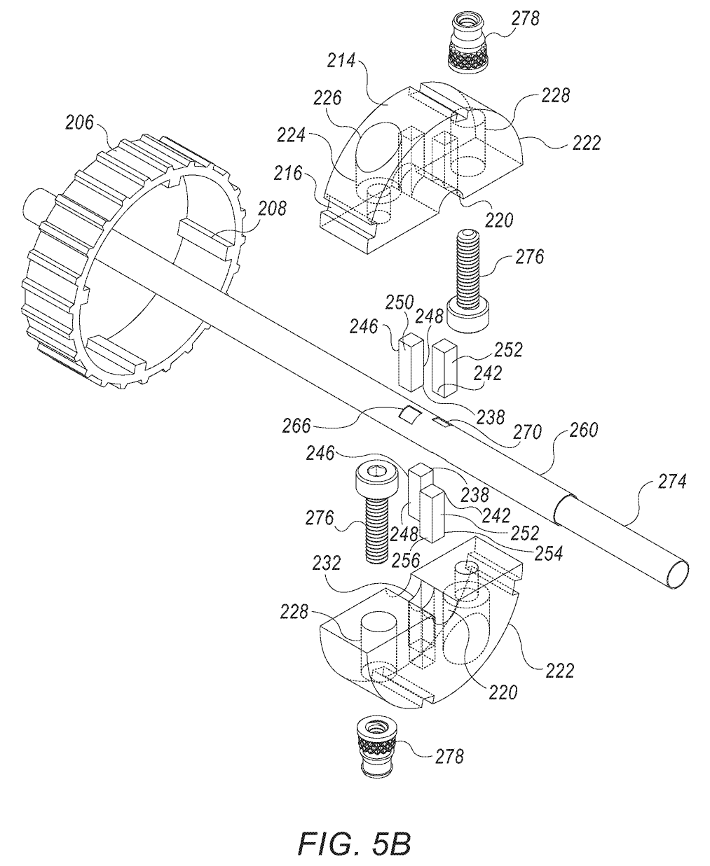

FIG. 5A is a perspective sectional view of an exemplary rotation control assembly.

FIG. 5B is a perspective exploded view of the assembly of FIG. 5A.

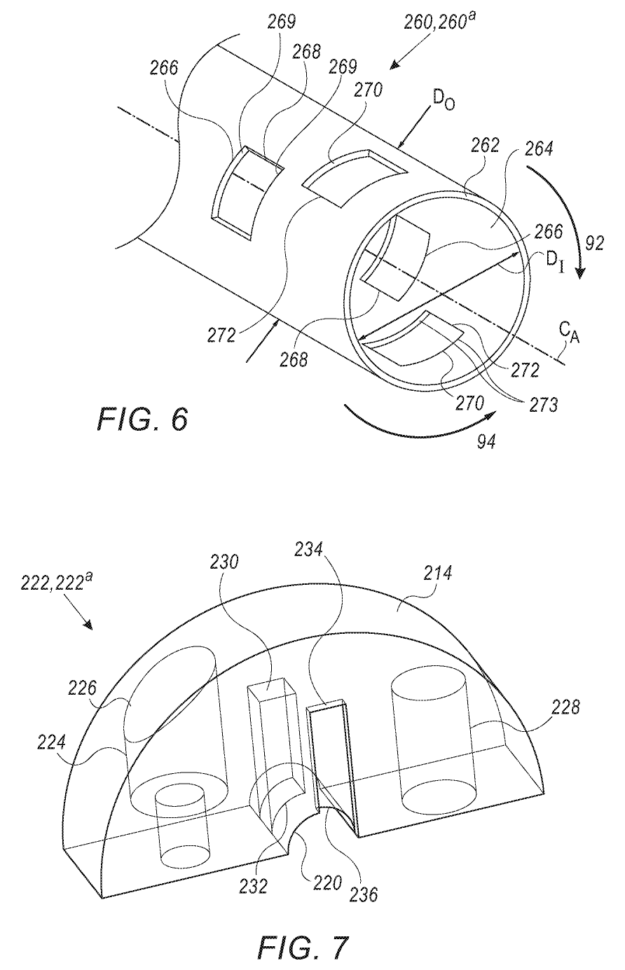

FIG. 6 is a perspective view of a proximal portion of an exemplary thin-walled tube that can be engaged by the assembly of FIGS. 5A-5B.

FIG. 7 is a perspective sectional view of a body portion of the assembly of FIGS. 5A-5B, shown with transparency.

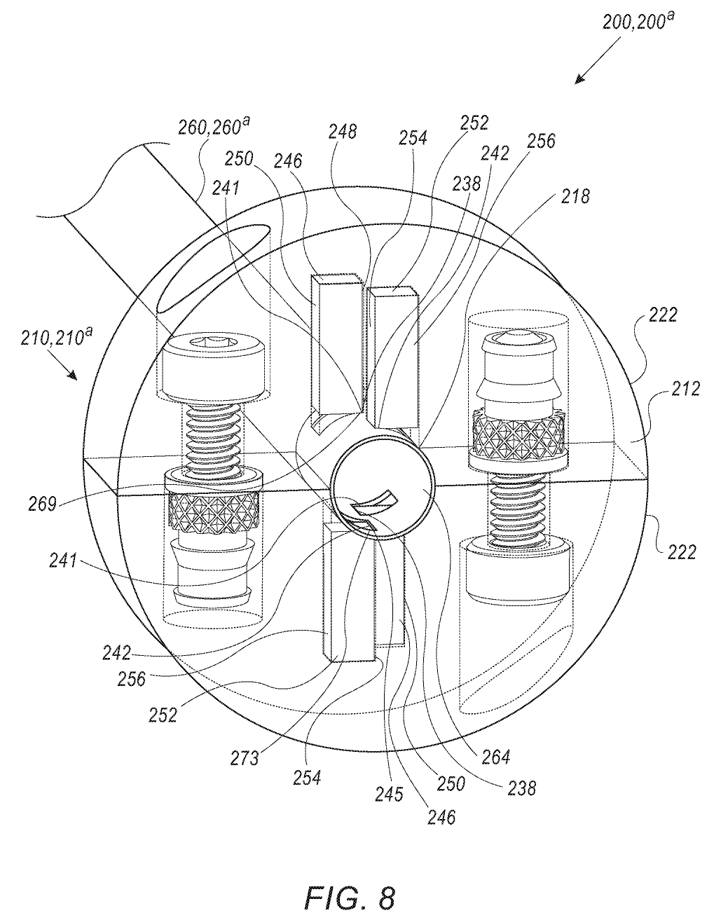

FIG. 8 is a perspective view of the assembly of FIGS. 5A-5B, with a rotatable body thereof shown with transparency.

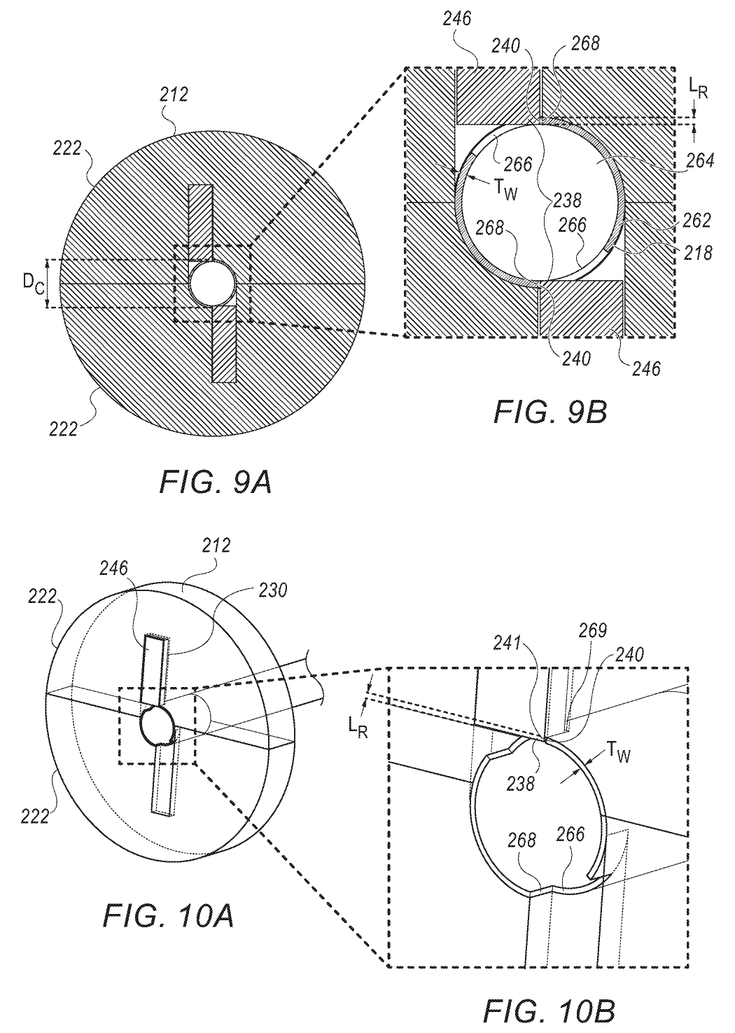

FIG. 9A is a cross-sectional view of the assembly of FIGS. 5A-5B.

FIG. 9B is an enlarged view of region 9B of FIG. 9A.

FIG. 10A is a perspective sectional view of the assembly of FIGS. 5A-5B.

FIG. 10B is an enlarged view of region 10B of FIG. 10A.

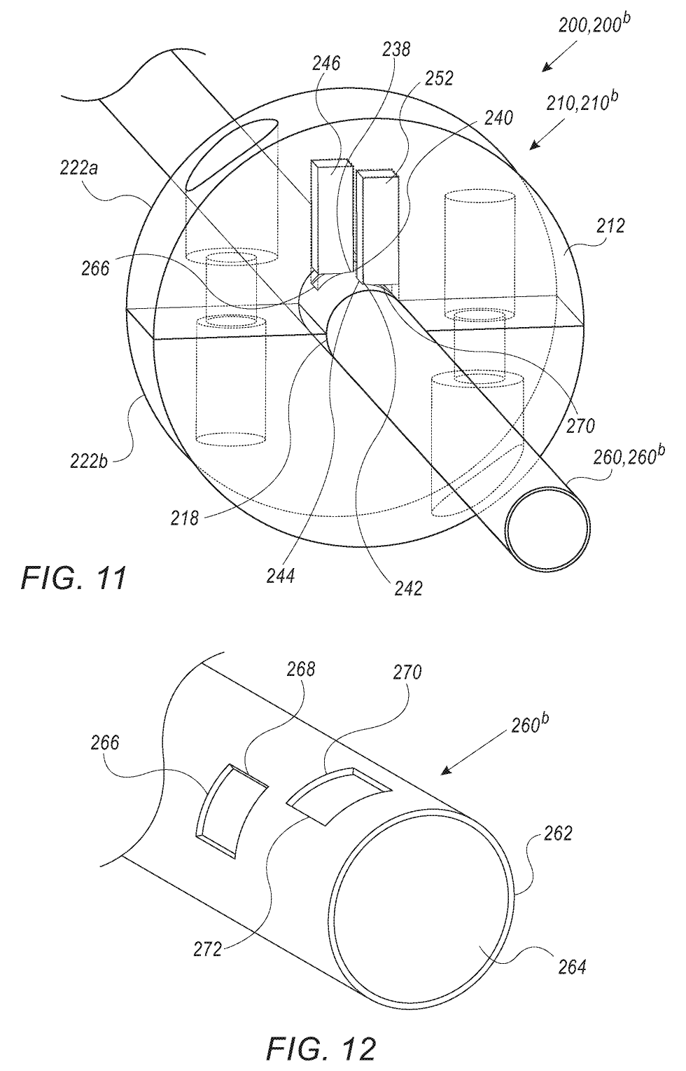

FIG. 11 is a perspective view of an exemplary rotation control assembly including two lugs that are axially offset from each other but having contact surfaces which are circumferentially aligned with each other.

FIG. 12 is a perspective view of a proximal portion of an exemplary thin-walled tube that can be used with the assembly of FIG. 11.

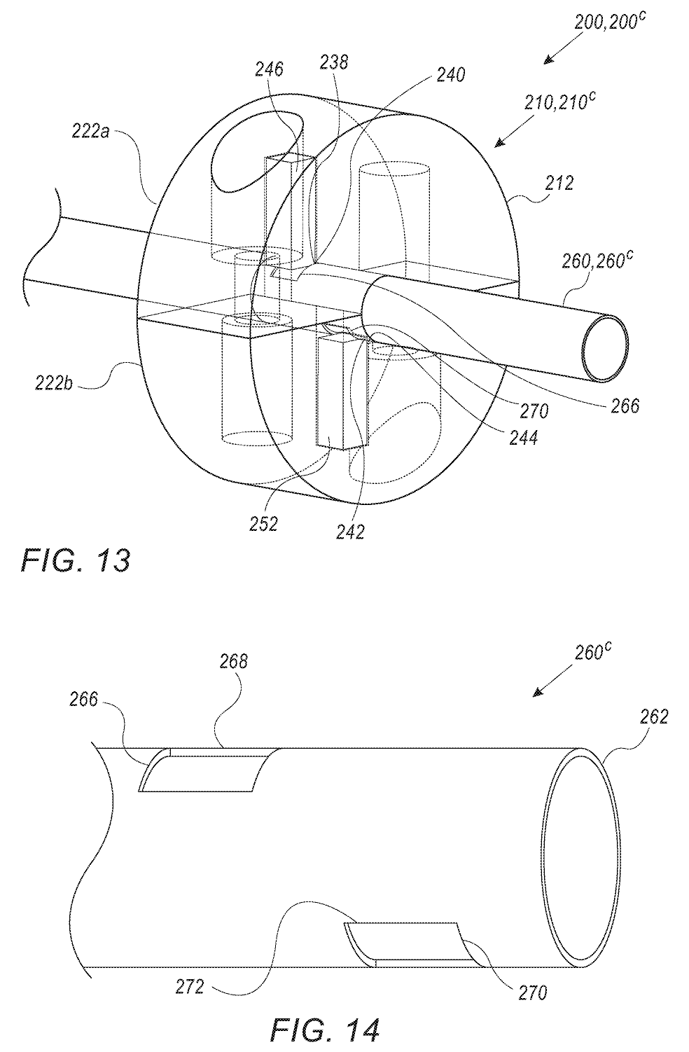

FIG. 13 is a perspective view of an exemplary rotation control assembly including two lugs that are axially and circumferentially offset from each other.

FIG. 14 is a perspective view of a proximal portion of an exemplary thin-walled tube that can be used with the assembly of FIG. 13.

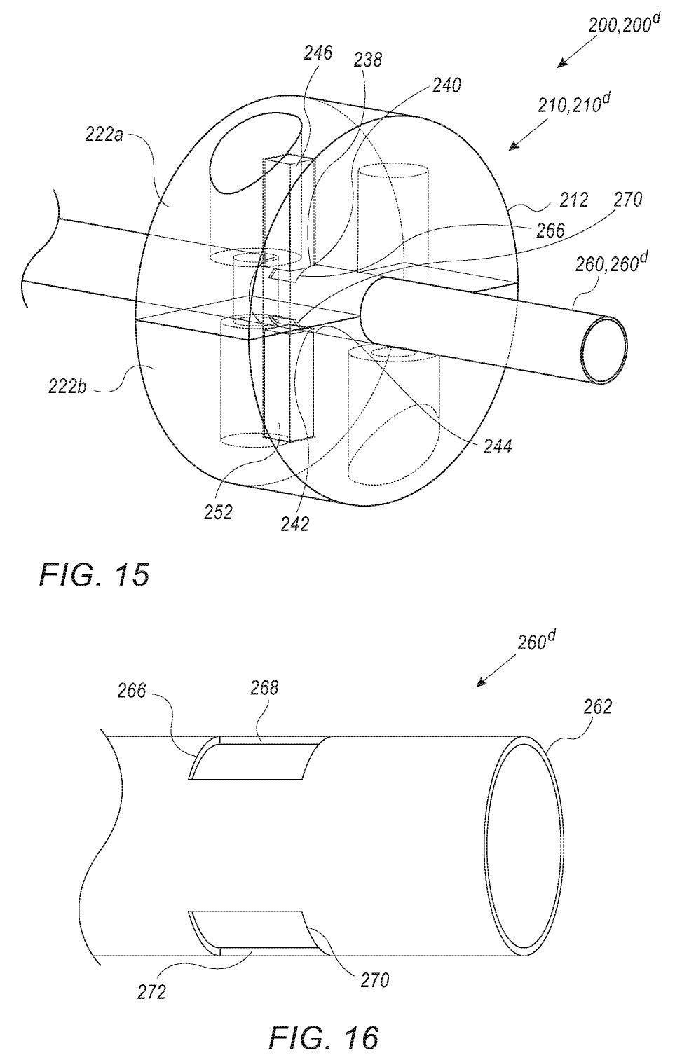

FIG. 15 is a perspective view of an exemplary rotation control assembly including two lugs that are axially aligned but circumferentially offset from each other.

FIG. 16 is a perspective view of a proximal portion of an exemplary thin-walled tube that can be used with the assembly of FIG. 15.

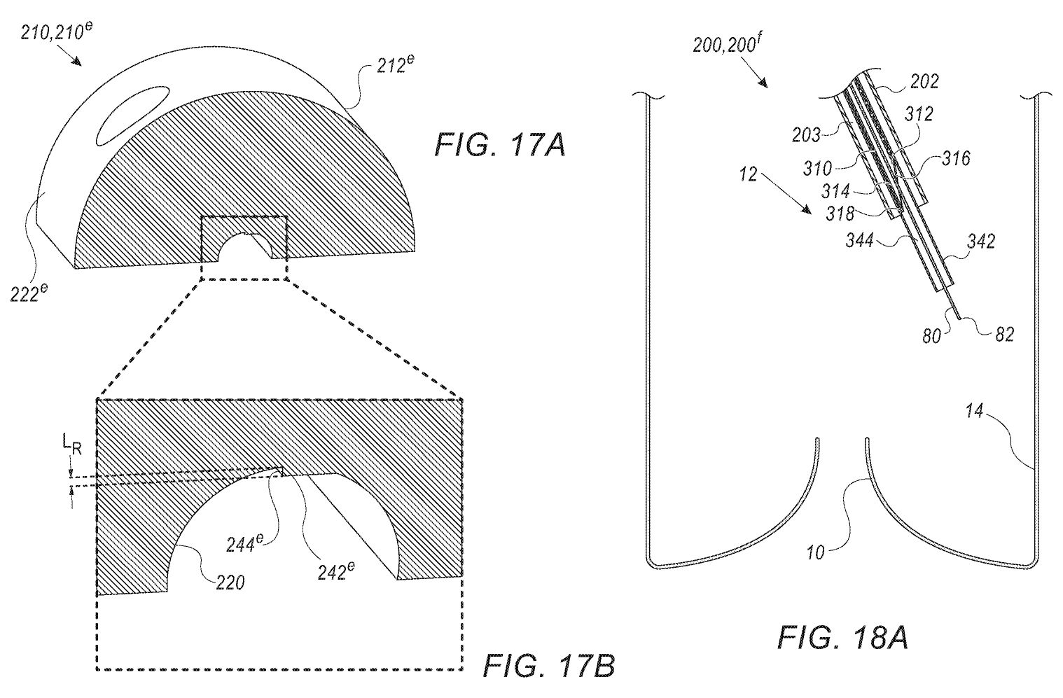

FIG. 17A is a perspective sectional view of a portion of an exemplary rotation control assembly having radial protrusions integrally formed with the rotatable body.

FIG. 17B shows an enlarged view of region 17B of FIG. 17A.

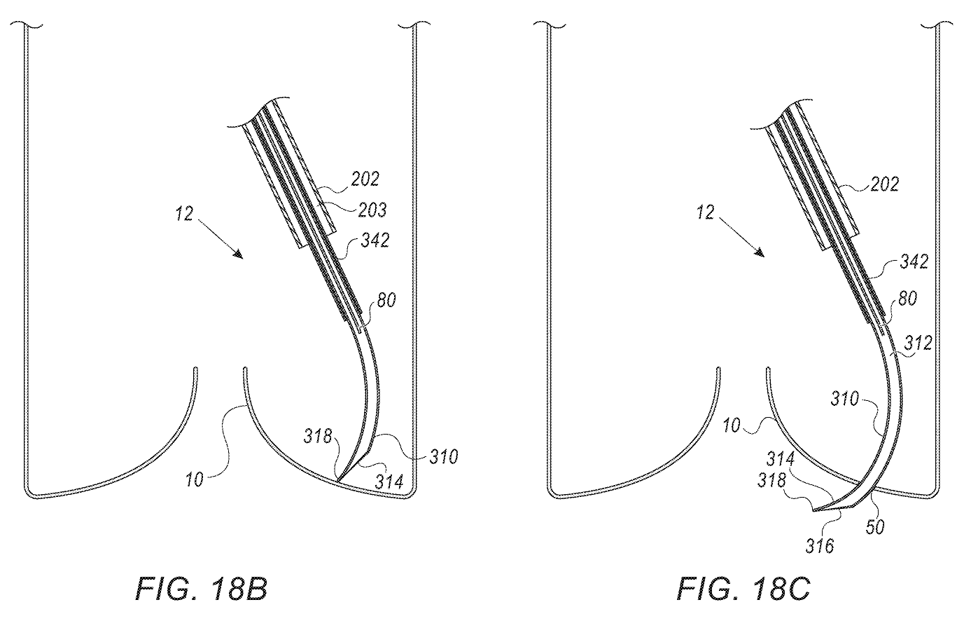

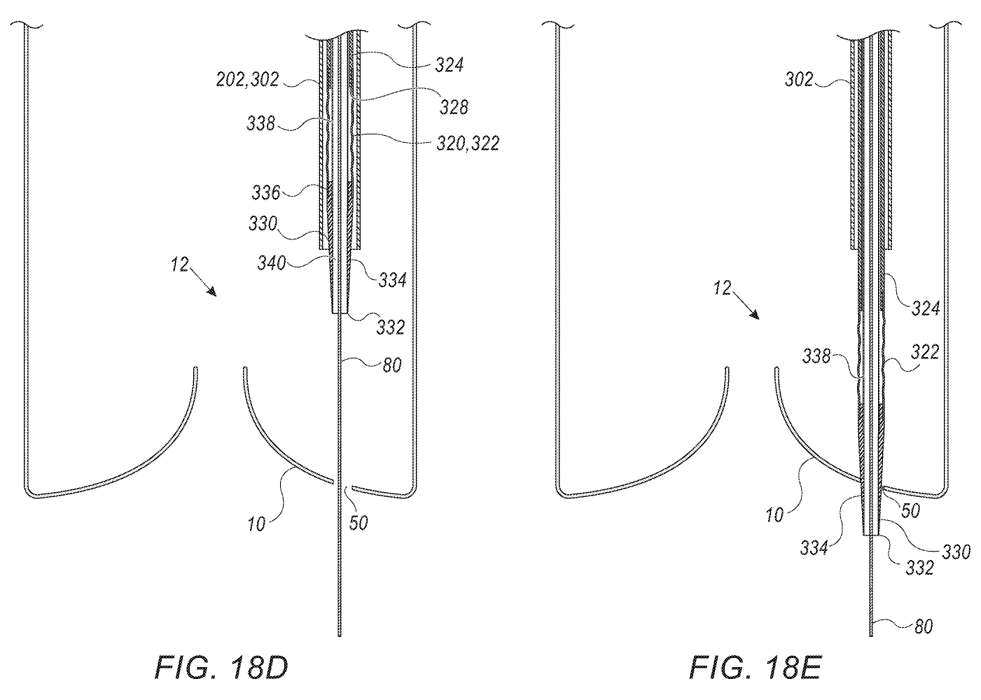

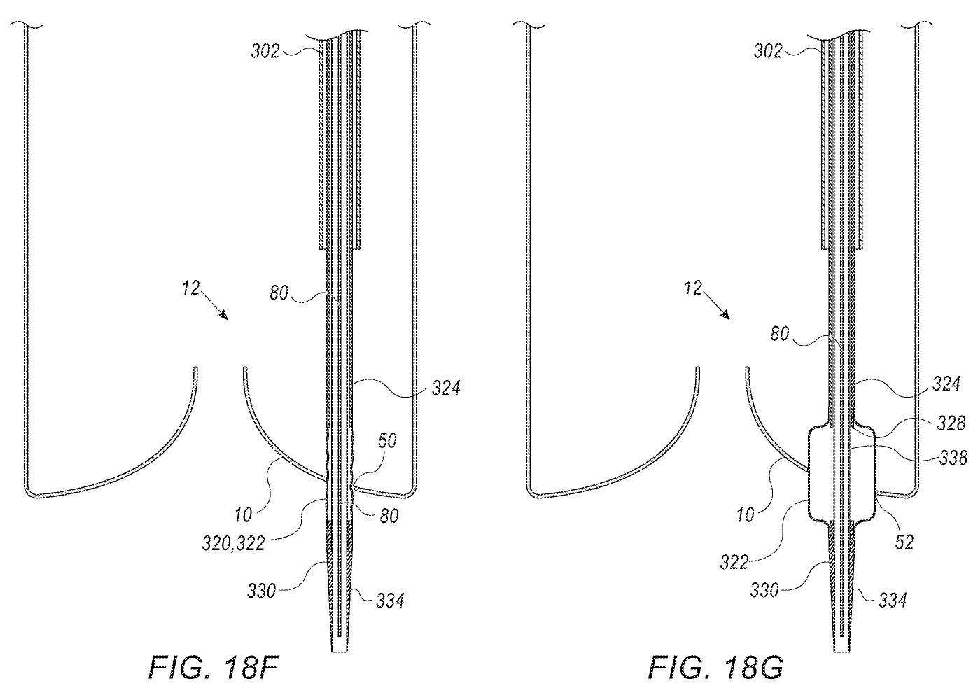

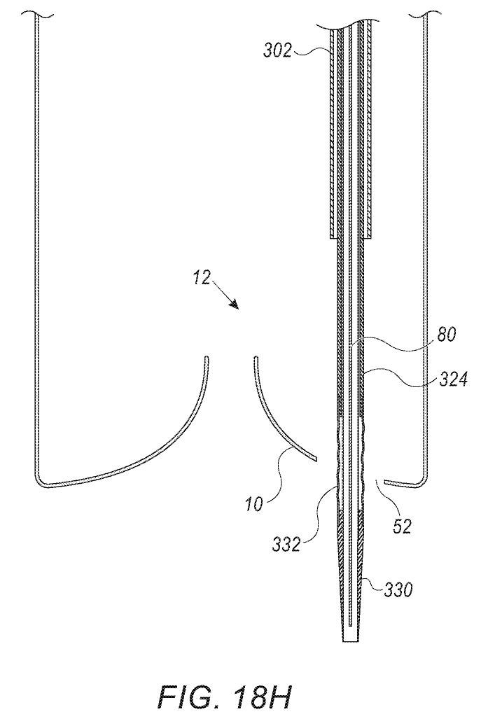

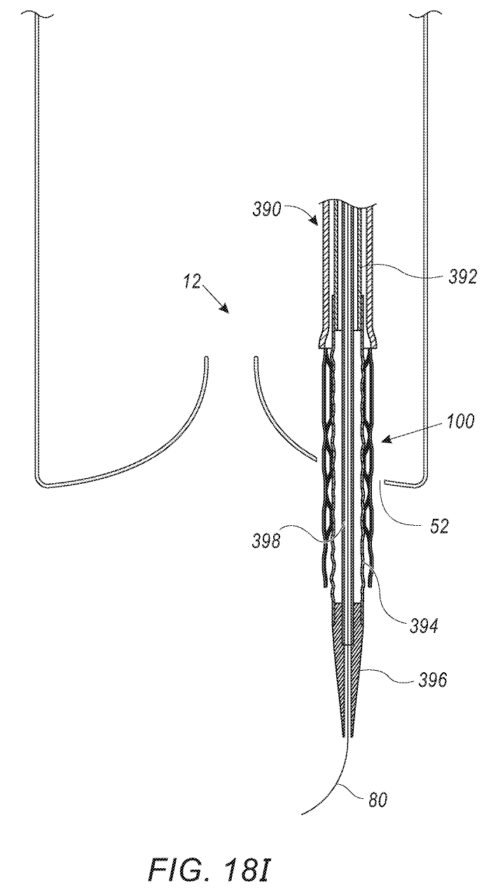

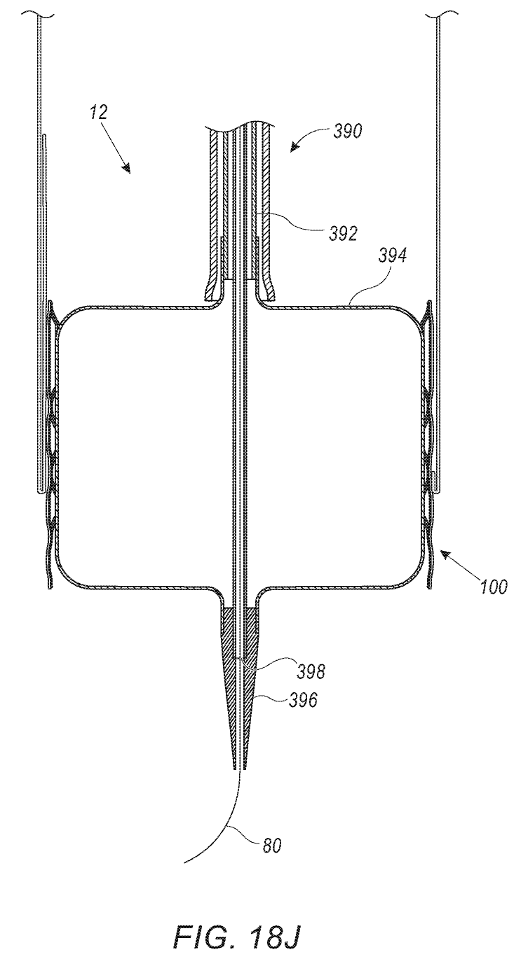

FIGS. 18A-18C illustrate steps in a method for utilizing a system that includes a pre-shaped bendable needle for forming a pilot puncture within a host leaflet.

FIGS. 18D-18H illustrate steps in a method for utilizing a hole-dilating balloon to dilate the pilot puncture to form a leaflet opening.

FIGS. 18I-18J illustrate steps in a method for positioning and expanding a guest prosthetic valve inside the leaflet opening.

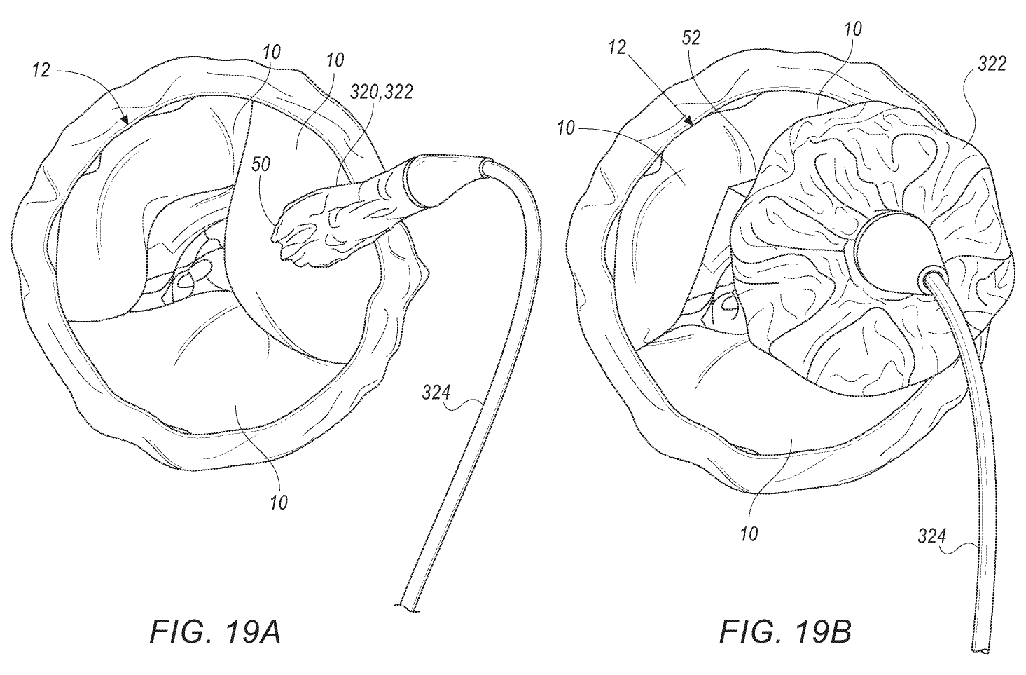

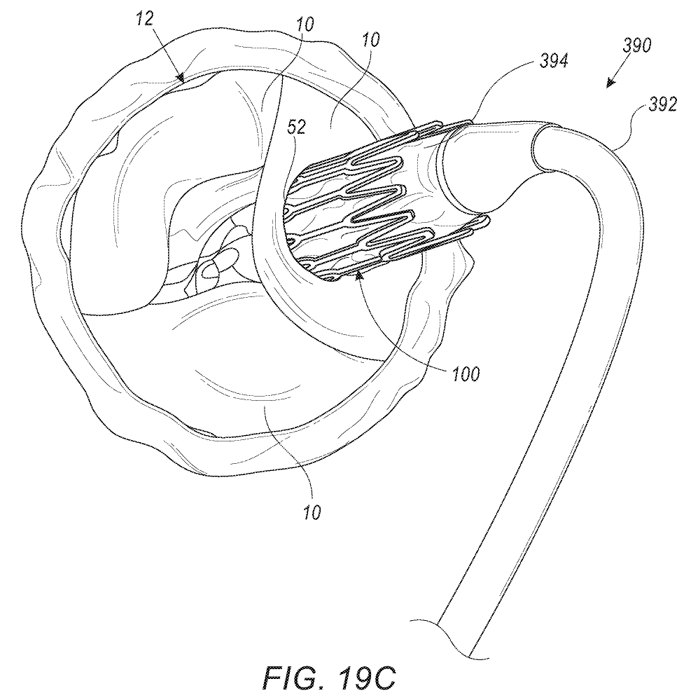

FIG. 19A shows the hole-dilation balloon positioned within a pilot puncture of the host leaflet in a deflated state.

FIG. 19B shows the hole-dilation balloon of FIG. 10A inflated within the host leaflet.

FIG. 19C shows the guest prosthetic valve positioned in the leaflet opening after removal of the hole-dilating balloon of FIG. 19B.

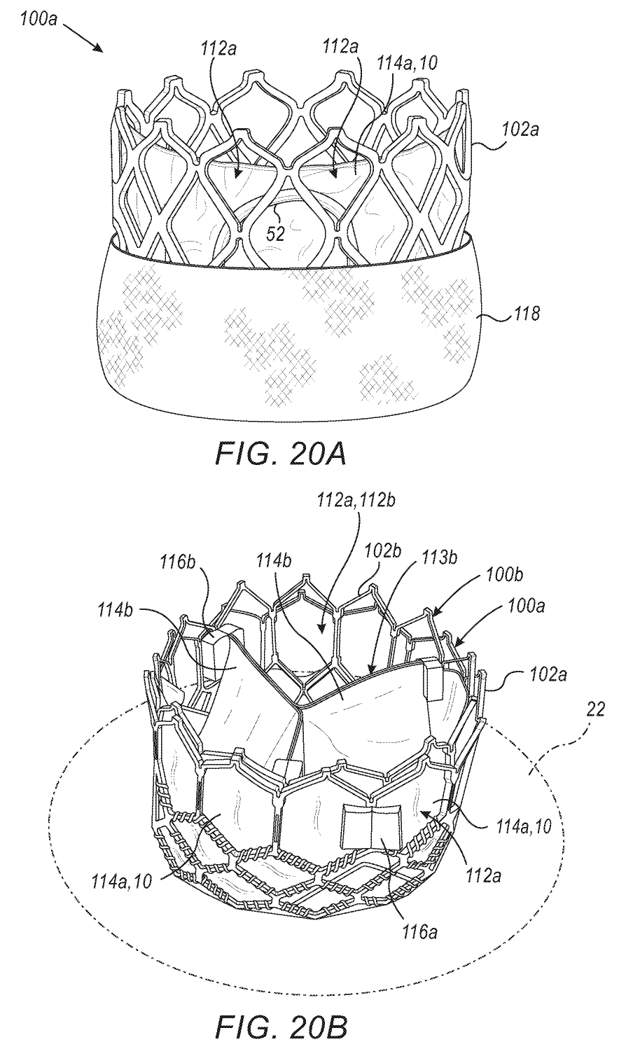

FIG. 20A is a perspective view of a host prosthetic valve subsequent to forming a leaflet opening thereof.

FIG. 20B is a perspective view of a guest prosthetic valve expanded within a leaflet opening of a host prosthetic valve.

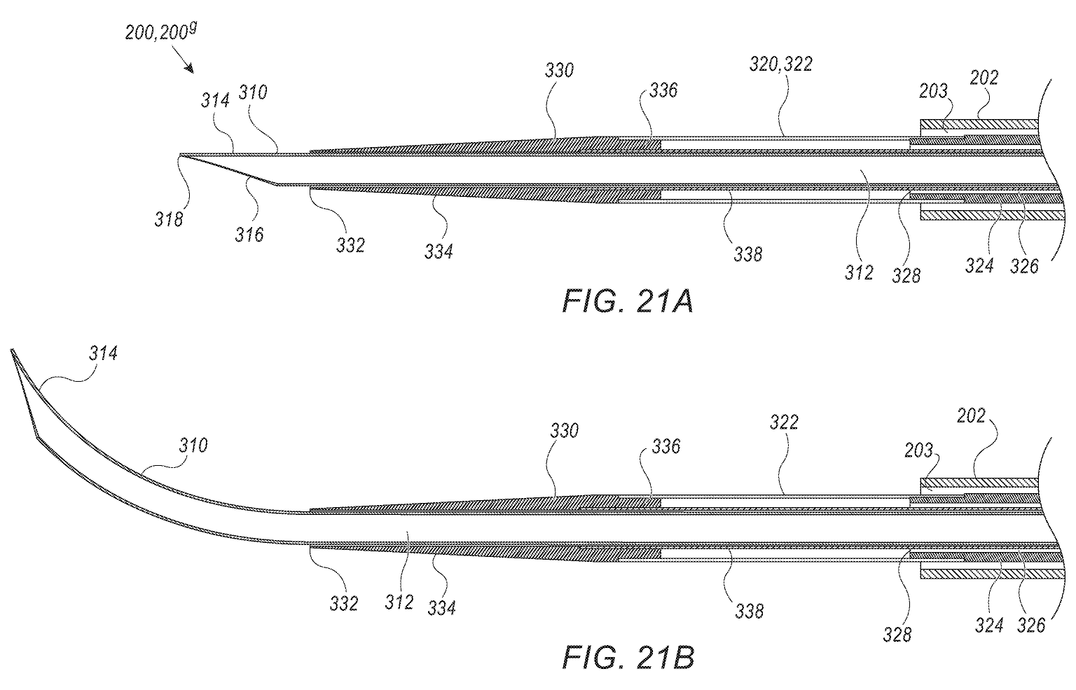

FIG. 21A is a cross-sectional view of a distal portion of a system having a needle extending through a dilator lumen, in an unbent state of the needle.

FIG. 21B is a cross-sectional view of a distal portion of the system of FIG. 21A, in a bent state of the needle.

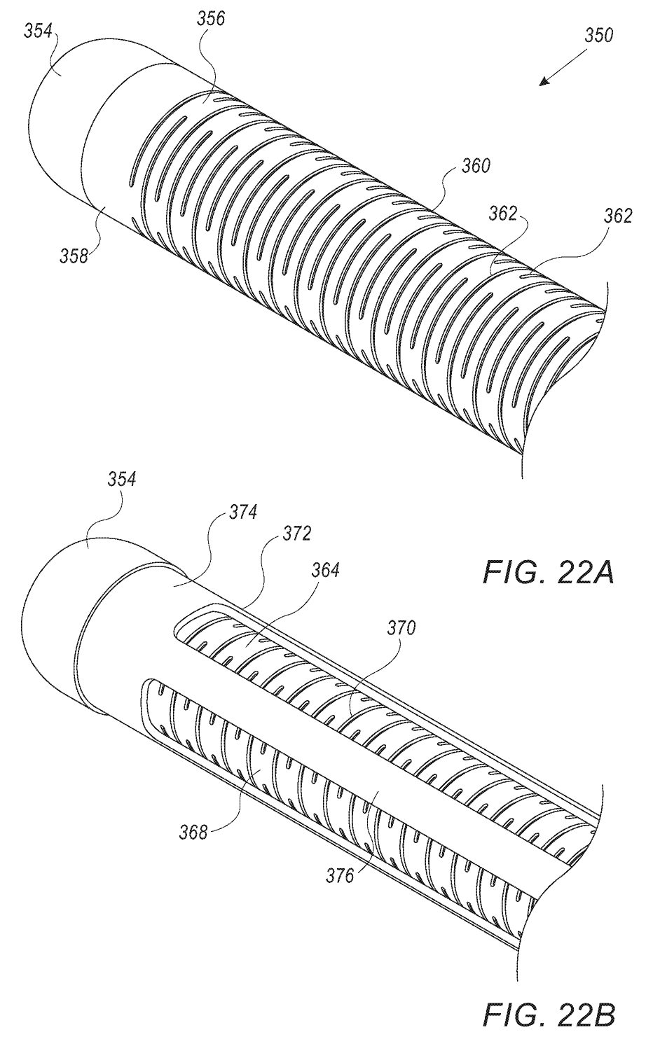

FIG. 22A is a perspective view of a distal portion of the exemplary steerable tube assembly.

FIG. 22B is a perspective view of a distal portion of the steerable tube assembly of FIG. 22A, with an outer bendable tube thereof removed from view.

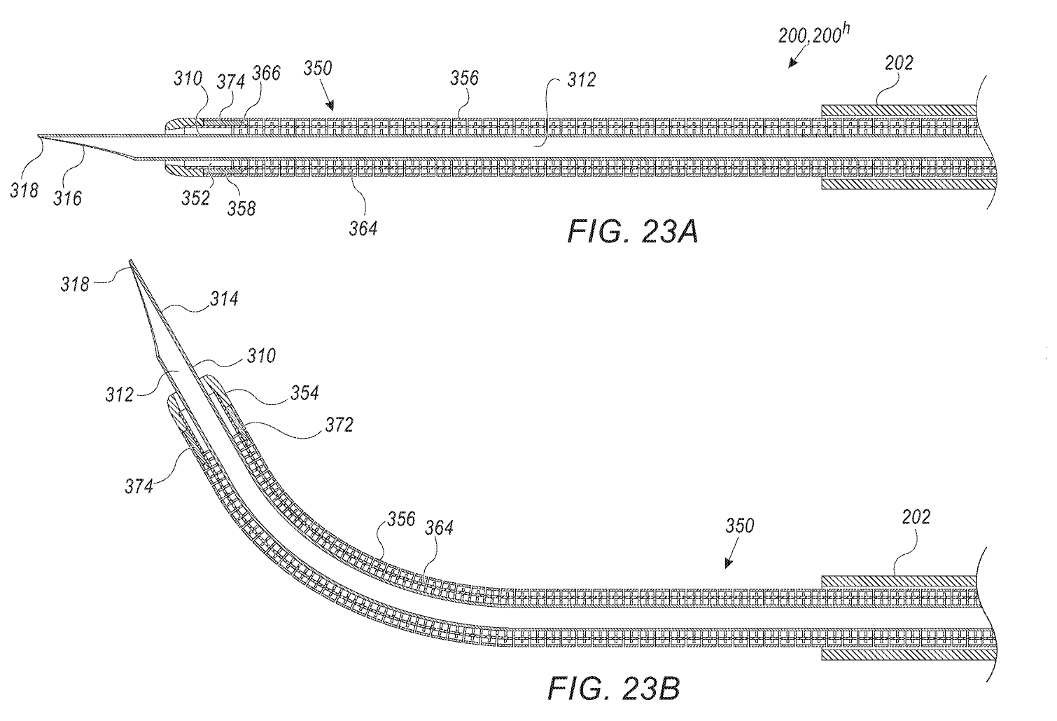

FIG. 23A is a cross-sectional view of a distal portion of a system comprising the steerable tube assembly of FIGS. 22A-22B, in an unbent state of the steerable tube assembly.

FIG. 23B is a cross-sectional view of a distal portion of the system of FIG. 23A, in a bent state of the steerable tube assembly.

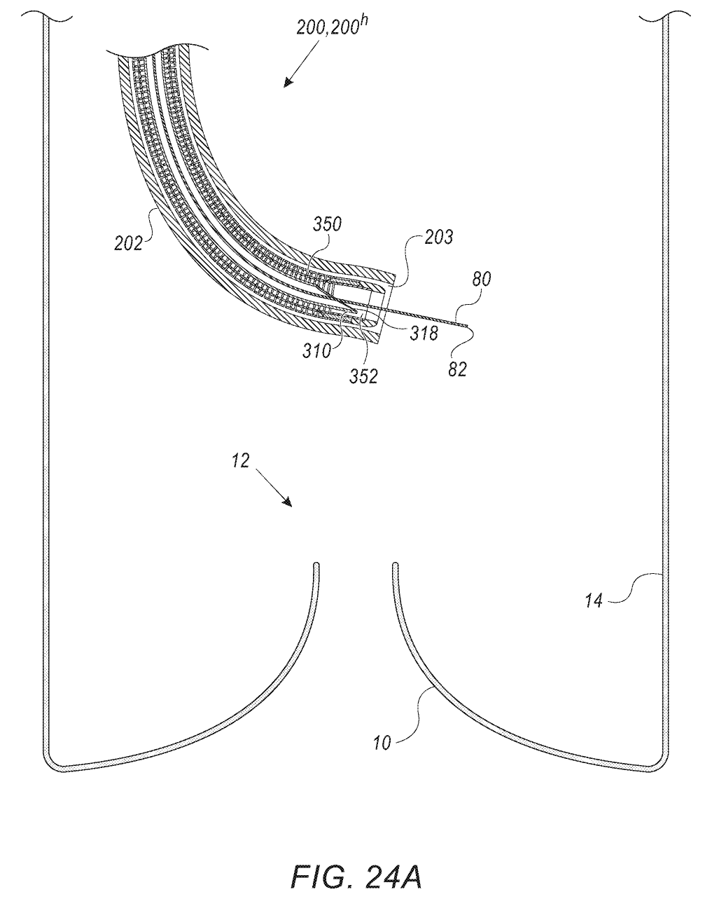

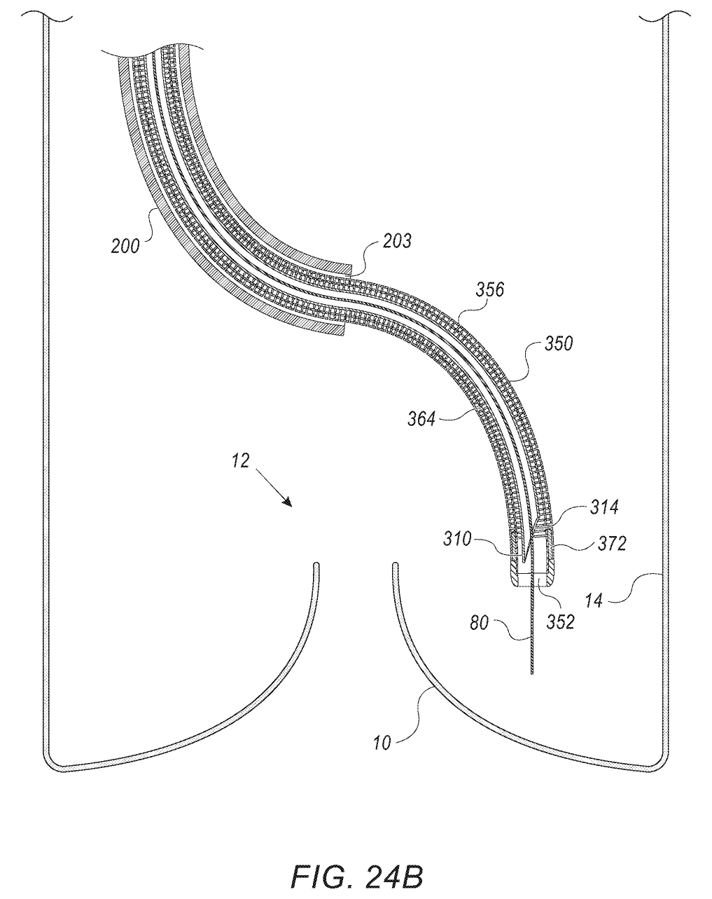

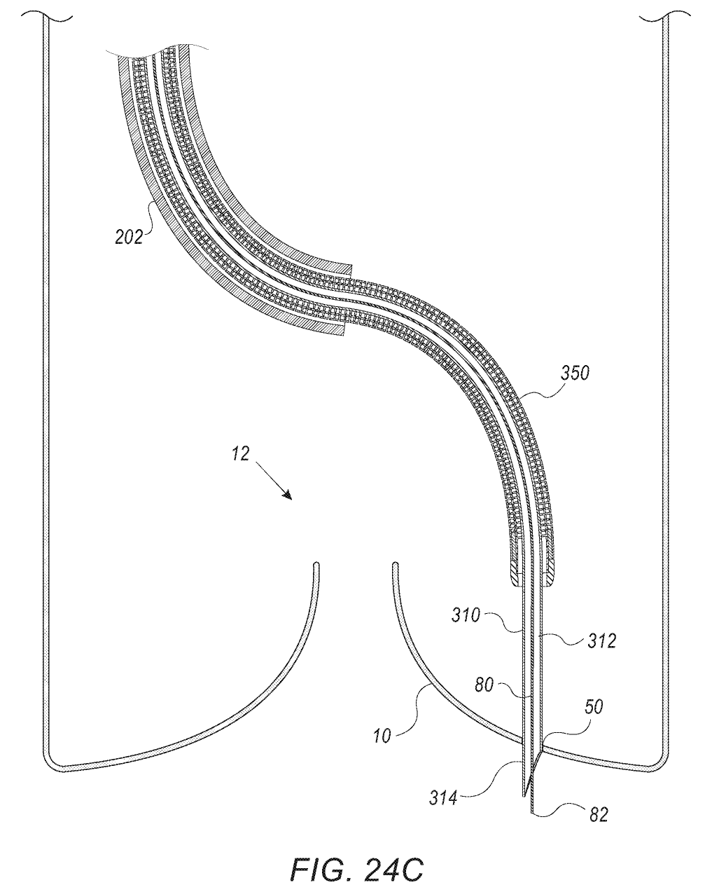

FIGS. 24A-24C illustrate steps in a method for utilizing the system of FIGS. 23A-23B for forming a pilot puncture within a host leaflet.

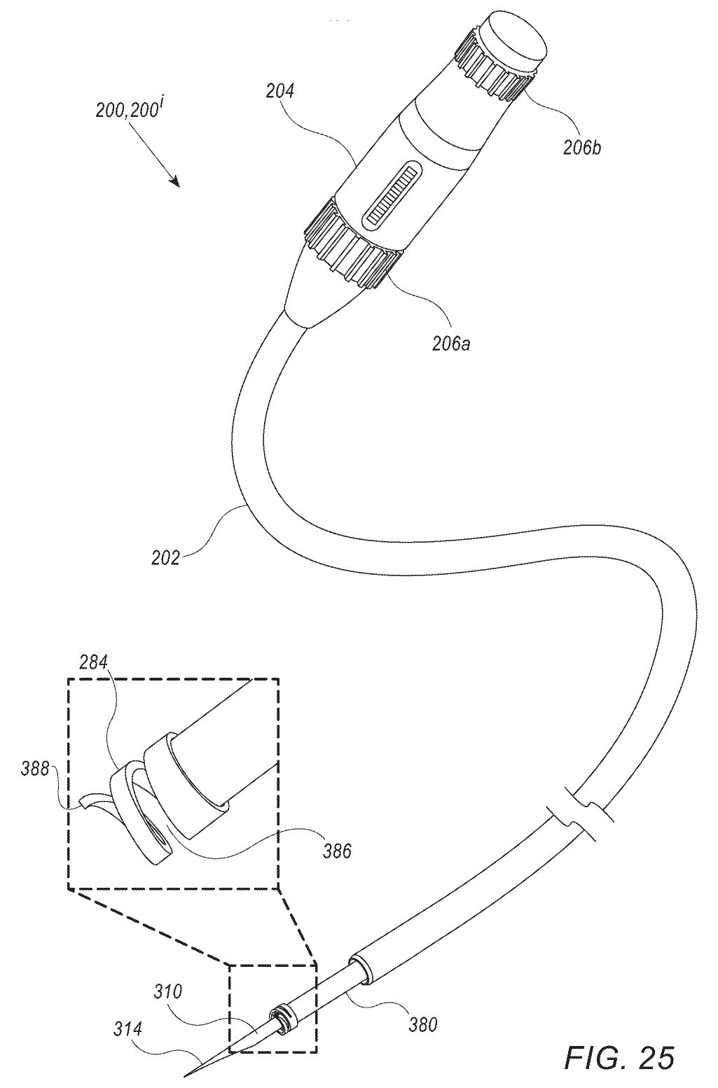

FIG. 25 shows an exemplary system comprising a helical anchor at a distal end of an anchor shaft.

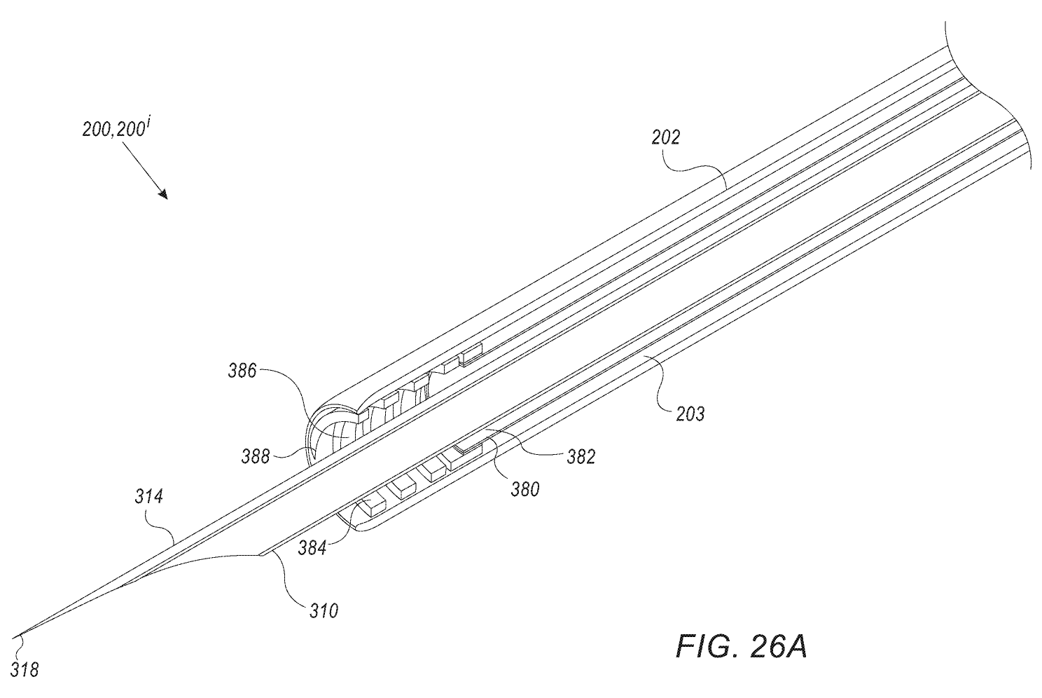

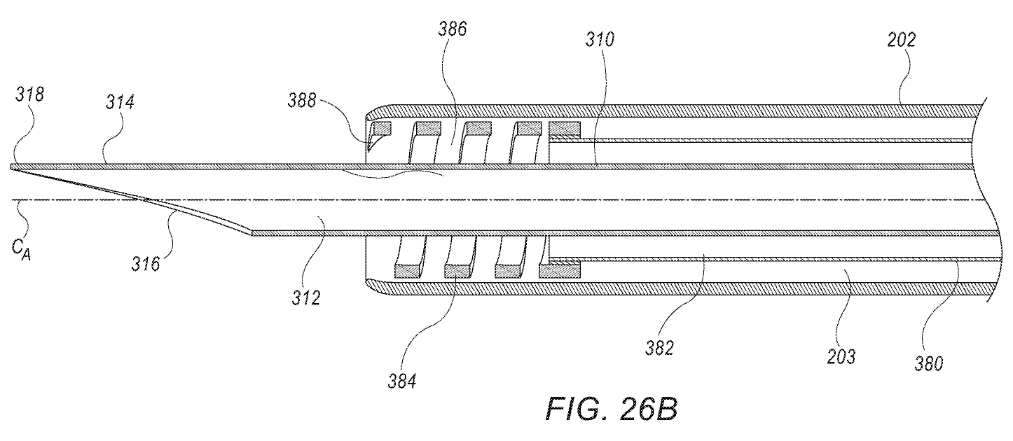

FIG. 26A is a perspective sectional view of a distal portion of the system of FIG. 25.

FIG. 26B is a cross-sectional side view of a distal portion of the system of FIG. 26A.

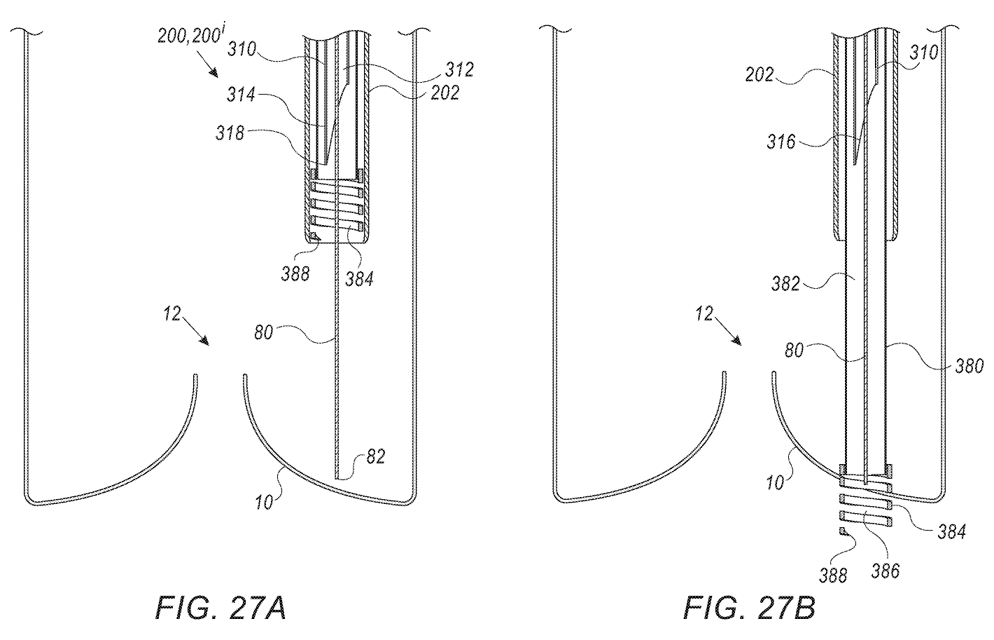

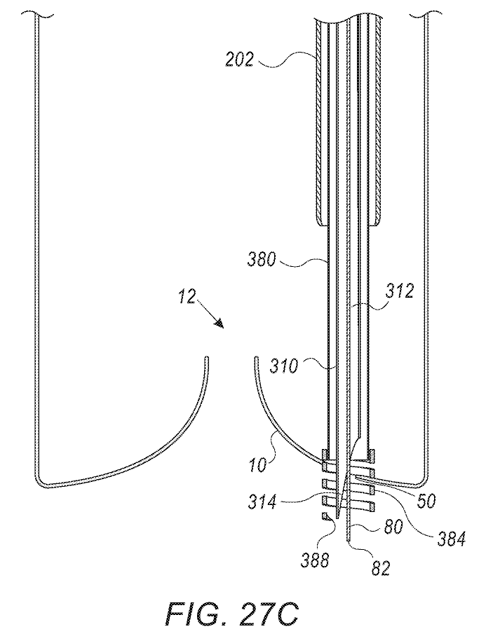

FIGS. 27A-27C illustrate steps in a method for utilizing the system of FIGS. 25-26B for forming a pilot puncture within a host leaflet.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like "provide" or "achieve" to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms "a", "an", and "the" include the plural forms unless the context clearly dictates otherwise. Additionally, the terms "have" or "includes" means "comprises". Further, the terms "coupled", "connected", and "attached", as used herein, are interchangeable and generally mean physically, mechanically, chemically, magnetically, and/or electrically coupled or linked and does not exclude the presence of intermediate elements between the coupled or associated items absent specific contrary language. As used herein, "and/or" means "and" or "or", as well as "and" and "or".

Directions and other relative references may be used to facilitate discussion of the drawings and principles herein, but are not intended to be limiting. For example, certain terms may be used such as "inner", "outer", "upper", "lower", "inside", "outside", "top", "bottom", "interior", "exterior", "left", right", and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an "upper" part can become a "lower" part simply by turning the object over. Nevertheless, it is still the same part and the object remains the same.

The term "plurality" or "plural" when used together with an element means two or more of the element. Directions and other relative references (for example, inner and outer, upper and lower, above and below, left and right, and proximal and distal) may be used to facilitate discussion of the drawings and principles herein but are not intended to be limiting.

The terms "proximal" and "distal" are defined relative to the use position of a delivery apparatus. In general, the end of the delivery apparatus closest to the user of the apparatus is the proximal end, and the end of the delivery apparatus farthest from the user (for example, the end that is inserted into a patient’s body) is the distal end. The term "proximal" when used with two spatially separated positions or parts of an object can be understood to mean closer to or oriented towards the proximal end of the delivery apparatus. The term "distal" when used with two spatially separated positions or parts of an object can be understood to mean closer to or oriented towards the distal end of the delivery apparatus. The terms "longitudinal" and "axial" are interchangeable, and refer to an axis extending in the proximal and distal directions, unless otherwise expressly defined.

The terms "axial direction", "radial direction", and "circumferential direction" have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms "integrally formed" and "unitary" refer to a construction that does not include any welds, fasteners, or other means for securing separately formed pieces of material to each other.

As used herein, operations that occur "simultaneously" or "concurrently" occur generally at the same time as one another, although delays in the occurrence of operation relative to the other due to, for example, spacing between components, are expressly within the scope of the above terms, absent specific contrary language.

As used herein, terms such as "first", "second", and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term "substantially" means the listed value and/or property and any value and/or property that is at least 75% of the listed value and/or property. Equivalently, the term "substantially" means the listed value and/or property and any value and/or property that differs from the listed value and/or property by at most 25%. For example, "at least substantially parallel" refers to directions that are fully parallel, and to directions that diverge by up to 22.5 degrees.

In the present disclosure, a reference numeral that includes an alphabetic label (for example, "a", "b", "c", etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

Described herein are steerable delivery catheters and related methods, which can be used to deliver tools and prosthetic devices to a location within a body of a subject. In some examples, steerable delivery catheters described herein can be used to deliver tools for modifying leaflets of an existing valvular structure in a patient’s heart, and/or for implanting prosthetic valves. Prior to or during implantation of the prosthetic heart valve within the existing valvular structure, each device, such as a delivery apparatus that can optionally carry a prosthetic valve, can be provided in the ascending aorta of a patient and can be used to pierce, lacerate, slice, tear, cut or otherwise modify a leaflet or commissure of the existing valvular structure. In some examples, the existing valvular structure can be a native aortic valve (for example, normal or abnormal, such as bicuspid aortic valve (BAV)) or a prosthetic valve previously implanted in the native aortic valve.

The modification can avoid, or at least reduce the likelihood of, issues that leaflets of the existing valvular structure might otherwise cause once the prosthetic heart valve has been fully installed, for example, obstruction of blood flow to the coronary arteries, improper mounting due to a non-circular valve cross-section, and/or restricted access to the coronary arteries if subsequent intervention is required. While described with respect to aortic valve, it should be understood that the disclosed examples can be adapted to deliver devices, such as cutting tools and/or implantable prosthetic devices, to and/or in any of the native annuluses of the heart (for example, the aortic, pulmonary, mitral, and tricuspid annuluses), and can be used with any of various delivery approaches (for example, retrograde, antegrade, transseptal, transventricular, transatrial, etc.).

FIG. 1 illustrates an anatomy of the aortic root 22, which is positioned between the left ventricle 32 and the ascending aorta 26. The aortic root 22 includes a native aortic valve 20 having a native valvular structure 29 comprising a plurality of native leaflets 30. Normally, the native aortic valve 20 has three leaflets (only two leaflets are visible in the simplified illustration of FIG. 1), but aortic valves with fewer than three leaflets are possible. The leaflets 30 are supported at native commissures by the aortic annulus 24, which is a ring of fibrous tissue at the transition point between the left ventricle 32 and the aortic root 22. The leaflets 30 can cycle between open and closed positions (the closed position is shown in FIG. 1) to regulate flow of blood from the left ventricle 32 to the ascending aorta 26. Branching off the aortic root 22 are the coronary arteries 34, 36. The coronary artery ostia 42, 44 are the openings that connect the aortic root 22 to the coronary arteries 34, 36.

FIGS. 2A-2B show an exemplary prosthetic valve 100 that can be implanted in a native heart valve, such as the native aortic valve 20 of FIG. 1. The term "prosthetic valve", as used herein, refers to any type of a prosthetic valve deliverable to a patient's target site over a catheter, which is radially expandable and compressible between a radially compressed, or crimped, state, and a radially expanded state. Thus, the prosthetic valve can be crimped on or retained by an implant delivery apparatus (not shown) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve of the current disclosure (for example, prosthetic valve 100) may include any prosthetic valve configured to be mounted within the native aortic valve, the native mitral valve, the native pulmonary valve, and the native tricuspid valve.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (not shown). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Patent No. 10,603,165, International Application No. PCT/US2021/052745 and U.S. Provisional Application Nos. 63/085,947 and 63/209904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

FIGS. 2A-2B show an example of a prosthetic valve 100, which can be a balloon expandable valve or any other type of valve, illustrated in an expanded state. The prosthetic valve 100 can comprise an outflow end 106 and an inflow end 104. In some instances, the outflow end 106 is the proximal end of the prosthetic valve 100, and the inflow end 104 is the distal end of the prosthetic valve 100. Alternatively, depending for example on the delivery approach of the valve, the outflow end can be the distal end of the prosthetic valve, and the inflow end can be the proximal end of the prosthetic valve.

The term "outflow", as used herein, refers to a region of the prosthetic valve through which the blood flows through and out of the prosthetic valve 100.

The term "inflow", as used herein, refers to a region of the prosthetic valve through which the blood flows into the prosthetic valve 100.

In the context of the present application, the terms "lower" and "upper" are used interchangeably with the terms "inflow" and "outflow", respectively. Thus, for example, the lower end of the prosthetic valve is its inflow end and the upper end of the prosthetic valve is its outflow end.

In the context of the present application, the terms "lower" and "upper" are used interchangeably with the terms "distal to" and "proximal to", respectively. Thus, for example, a lowermost component can refer to a distal-most component, and an uppermost component can similarly refer to a proximal-most component.

The terms "longitudinal" and "axial", as used herein, refer to an axis extending in the proximal and distal directions, unless otherwise expressly defined.

The prosthetic valve 100 comprises an annular frame 102 movable between a radially compressed configuration and a radially expanded configuration, and a valvular structure 113 that comprises prosthetic valve leaflets 114 mounted within the frame 102. The frame 102 can be made of various suitable materials, including plastically-deformable materials such as, but not limited to, stainless steel, a nickel-based alloy (for example, a cobalt-chromium or a nickel-cobalt-chromium alloy such as MP35N alloy), polymers, or combinations thereof. When constructed of a plastically-deformable materials, the frame 102 can be crimped to a radially compressed state on a balloon catheter, and then expanded inside a patient by an inflatable balloon or equivalent expansion mechanism. Alternatively or additionally, the frame 102 can be made of shape-memory materials such as, but not limited to, nickel titanium alloy (for example, Nitinol). When constructed of a shape-memory material, the frame 102 can be crimped to a radially compressed state and restrained in the compressed state by insertion into a shaft or equivalent mechanism of a delivery apparatus.

In the example illustrated in FIGS. 2A-2B, the frame 102 is an annular, stent-like structure comprising a plurality of intersecting struts 108. In this application, the term "strut" encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 108 may be any elongated member or portion of the frame 102. The frame 102 can include a plurality of strut rungs that can collectively define one or more rows of cells 110. The frame 102 can have a cylindrical or substantially cylindrical shape having a constant diameter from the inflow end 104 to the outflow end 106 as shown, or the frame can vary in diameter along the height of the frame, as disclosed in US Pat. No. 9,155,619, which is incorporated herein by reference.

The struts 108 can include a plurality of angled struts and vertical or axial struts. At least some of the struts 108 can be pivotable or bendable relative to each other, so as to permit frame expansion or compression. For example, the frame 102 can be formed from a single piece of material, such as a metal tube, via various processes such as, but not limited to, laser cutting, electroforming, and/or physical vapor deposition, while retaining the ability to collapse/expand radially in the absence of hinges and like.

A valvular structure 113 of the prosthetic valve 100 can include a plurality of prosthetic valve leaflets 114 (for example, three leaflets), positioned at least partially within the frame 102, and configured to regulate flow of blood through the prosthetic valve 100 from the inflow end 104 to the outflow end 106. While three leaflets 114 arranged to collapse in a tricuspid arrangement, are shown in the example illustrated in FIGS. 2A-2B, it will be clear that a prosthetic valve 100 can include any other number of leaflets 114. Adjacent leaflets 114 can be arranged together to form prosthetic valve commissures 116 that are coupled (directly or indirectly) to respective portions of the frame 102, thereby securing at least a portion of the valvular structure 113 to the frame 102. The prosthetic valve leaflets 114 can be made from, in whole or part, biological material (for example, pericardium), bio-compatible synthetic materials, or other such materials. Further details regarding transcatheter prosthetic valves, including the manner in which leaflets 114 can be coupled to the frame 102 of the prosthetic valve 100, can be found, for example, in U.S. Patent Nos. 6,730,118, 7,393,360, 7,510,575, 7,993,394, 8,652,202, and 11,135,056, all of which are incorporated herein by reference in their entireties.

In some examples, the prosthetic valve 100 can comprise at least one skirt or sealing member. For example, the prosthetic valve 100 can include an inner skirt (not shown in FIG. 2A-2B), which can be secured to the inner surface of the frame 102. Such an inner skirt can be configured to function, for example, as a sealing member to prevent or decrease perivalvular leakage. An inner skirt can further function as an anchoring region for leaflets 114 to the frame 102, and/or function to protect the leaflets 114 against damage which may be caused by contact with the frame 102, for example during valve crimping or during working cycles of the prosthetic valve 100. An inner skirt can be disposed around and attached to the inner surface of frame 102, while the leaflets can be sutured to the inner skirt along a scalloped line (not shown). An inner skirt can be coupled to the frame 102 via sutures or another form of coupler.

The prosthetic valve 100 can comprise, in some examples, an outer skirt 118 mounted on the outer surface of frame 102 (as shown in FIGS. 2A-2B), configured to function, for example, as a sealing member retained between the frame 102 and the surrounding tissue of the native annulus against which the prosthetic valve is mounted, or against an inner side of a previously implanted valve in the case of ViV procedures (described further below), thereby reducing risk of paravalvular leakage (PVL) past the prosthetic valve 100. The outer skirt 118 can be coupled to the frame 102 via sutures or another form of coupler.

Any of the inner skirt and/or outer skirt can be made of various suitable biocompatible materials, such as, but not limited to, various synthetic materials (for example, PET) or natural tissue (for example pericardial tissue). In some cases, the inner skirt can be formed of a single sheet of material that extends continuously around the inner surface of frame 102. In some cases, the outer skirt 118 can be formed of a single sheet of material that extends continuously around the outer surface of frame 102.

The cells 110, defined by interconnected struts 108, define cell openings 112. While some of the cell openings 112 can be covered by the inner skirt and/or the outer skirt, at least a portion of the cell opening 112 can remain uncovered, such as cell openings 112 which are closer to the outflow end 106 of the prosthetic valve.

FIGS. 2A-2B illustrate a hypothetical coronary artery obstruction that could occur in some cases from implantation of a prosthetic valve 100 within the native aortic valve 20. In this example, the prosthetic valve 100 is the guest valve or new valve, and the native aortic valve 20 is the host valve or old valve.

During implantation of the prosthetic valve 100, the prosthetic valve 100 is positioned within a central region defined between the native leaflets 30, which are also the host leaflets 10 for the example illustrated in FIG. 2A-2B. The prosthetic valve 100 is then radially expanded against the host leaflets 10. As illustrated, the host leaflets 10 form a tube around the frame 102 of the prosthetic valve 100 after the prosthetic valve 100 is radially expanded to the working diameter. As further illustrated, expansion of the prosthetic valve 100 displaces the host leaflets 10 outwards towards the coronary ostia 42, 44 such that the host leaflets 10 contact a portion of the aortic root 22 surrounding the coronary ostia 42, 44, causing coronary artery obstruction.

For an existing implanted prosthetic valve, the valvular structure may naturally degrade over time thereby requiring repair or replacement in order to maintain adequate heart functions. In a Valve-in-Valve (ViV) procedure, a new prosthetic heart valve is mounted within the existing, degrading prosthetic heart valve in order to restore proper function. FIG. 3 illustrates an exemplary hypothetical coronary artery obstruction that could occur in some cases from implantation of a prosthetic valve 100b within a previously implanted prosthetic valve 100a (for example, after a ViV procedure). In this example, the prosthetic valve 100b is the guest valve or new valve, and the prosthetic valve 100a is the host valve or old valve. In this example, the prosthetic valve 100a was previously implanted within the orifice of the native aortic valve 20. Each of the prosthetic valves 100a, 100b can have the general structure of the prosthetic valve 100 described with reference to FIGS. 2A-2B, though in some examples, each of the prosthetic valves 100a, 100b can be a different type of prosthetic valve. For example, a balloon expandable guest valve 100b can be implanted inside a previously implanted mechanically expandable or self-expandable host valve 100a.

During implantation of the prosthetic valve 100b, the prosthetic valve 100b is positioned within a central region defined between the leaflets 114a of the prosthetic valve 100a, which now take the role of host leaflet 10. The prosthetic valve 100b is then radially expanded against the host leaflets 10 (i.e., against the prosthetic valve leaflets 114c). As illustrated, the radial expansion of the prosthetic valve 100a results in outward displacement of the host leaflets 10. As further illustrated, the host leaflets 10 are displaced such that the host leaflets 10 contact the aortic root 22 at positions superior to the coronary artery ostia 42, 44, causing coronary artery ostia obstruction. Alternatively, the guest prosthetic valve 100b can displace the host leaflets 114a outwardly against the frame 102a of the host valve 100a, thereby blocking the flow of blood through the frame 102a to the coronary ostia 42, 44.

In some patient anatomies (for example, when the outflow end 106 of the prosthetic valve 100 is at the STJ level 28 and the diameter of the prosthetic valve 100 is similar to the STJ diameter such that the frame 102 touches or is very close to the aortic wall 38 at the STJ level 28), the host leaflets 10 may compromise the ability for future access into the coronary arteries 34, 36 or perfusion through the frame 102 to the coronary arteries 34, 36 during the diastole phase of the cardiac cycle. Similar problems may occur in some patient anatomies either when a guest prosthetic valve 100b is percutaneously expanded within a previously implanted host prosthetic valve 100a, or when a prosthetic valve 100 is percutaneously expanded within a native valve, displacing the native leaflets 30 outward toward the coronary ostia 42, 44.

The risk illustrated in FIG. 3 may be higher when the host valve is a bioprosthetic valve without a frame or when the leaflets of the host valve are external to a frame. Risk of coronary artery ostia obstruction can increase in a cramped aortic root or when the coronary artery ostium sits low. In the examples illustrated in FIGS. 2A-3, the host leaflets 10 are shown obstructing both coronary artery ostia 42, 44. In some cases, only one host leaflet 10 may obstruct a respective coronary artery ostium. For example, the risk of obstructing the left coronary ostium 42 tends to be greater than obstructing the right coronary ostium 44 because the left coronary ostium 42 typically sits lower than the right coronary ostium 44.

The term "host valve" as used herein refers to a native heart valve in which a prosthetic valve is implanted or a previously implanted prosthetic valve in which a new prosthetic valve is implanted. Moreover, in any of the examples disclosed herein, when the host valve is a previously implanted prosthetic valve, the host valve can be a surgically implanted prosthetic heart valve (known as a "surgical valve") or a transcatheter heart valve. The term "guest valve", as used herein, refers to a prosthetic valve implanted in a host valve, which can be either a native heart valve or a previously implanted prosthetic valve. Similarly, the term "host leaflets 10", as used herein, refers to native leaflets 30 of a native valve in which a new guest prosthetic valve 100 is implanted, or to prosthetic valve leaflets 114a of a previously implanted host valve 100a in which a new guest prosthetic valve 100b is implanted.

When a guest prosthetic valve 100 is deployed inside a host valvular structure 12, it displaces the host leaflets 10 of the host valve radially outwards, towards and against a host interior surface 14, which can be the interior surface of the aortic wall 38 if the host valve is the native valve, or an interior surface of the frame 102a of a previously implanted prosthetic valve 100a serving as the host valve.

To avoid obstruction of blood flow to the coronary arteries 34, 36, the valvular structure 12 of the existing host valve (whether a native aortic valve or a previously implanted prosthetic valve) can be modified by components of a delivery apparatus prior to or during implantation of a new prosthetic valve within the existing valvular structure 12. In some examples, the host valvular structure 12 is modified by piercing, lacerating, tearing, slicing, and/or cutting one or more host leaflets 10 (for example, a free end of the host leaflet 10 or a commissure of adjacent host leaflets 10, which can be a native commissure 40 for a native aortic valve 20, or a prosthetic valve commissure 116 for a previously implanted host prosthetic valve 100) using the delivery apparatus. The modification thus disrupts the impermeable tubular structure that would otherwise be formed by the existing host leaflets 10, thereby allowing blood to flow to the coronary arteries 34, 36.

FIG. 4 illustrates an exemplary system 200 that includes a handle 204 and at least one tube 260 (shown, for example, in FIGS. 5A-6), which can be, in some examples, a thin-walled tube, extending distally from the handle 204. In some examples, the system 200 can include an outer shaft 202 extending distally from the handle 204, and the tube 260 can extend through the outer shaft 202. In some examples, the system 200 can include one or more components configured for cutting or lacerating a target tissue, such as for modifying a host leaflet. In some examples, the system 200 can include a perforating member 310 configured to puncture through a target tissue. In some examples, the perforating member 310 is in the form of a tubular member, such as a needle 310, as illustrated in FIG. 4, though it is to be understood that other examples of perforating members are contemplated. In some examples, the tube 260 of the system 200 is a hollow perforating member 310, such as a hollow needle. In some examples, a perforating member can be covered by, and optionally axially movable relative to, an overtube 342, as shown in the example illustrated in FIG. 4. In some examples, the tube 260 of the system 200 is the overtube 342 disposed over the perforating member 310.

The outer shaft 202, the perforating member 310, and when present, the overtube 342, can be configured to be axially movable relative to each other. For example, a distally oriented movement of the perforating member 310 relative to the outer shaft 202, and/or a proximally oriented movement of the outer shaft 202 relative to the perforating member 310, can expose a distal end portion 314 of the perforating member 310. The tube 260 can be configured to rotate relative to the outer shaft 202. In some examples, the outer shaft 202 and the tube 260 can be configured to be axially movable relative to each other. For example, a distally oriented movement of the tube 260 relative to the outer shaft 202, and/or a proximally oriented movement of the outer shaft 202 relative to the tube 260, can expose a distal portion of the tube 260.

The proximal ends of the outer shaft 202 and the perforating member 310, as well as an optional overtube 342, can be coupled to the handle 204. During advancement through a patient's vasculature, the handle 204 can be maneuvered by an operator (for example, a clinician or a surgeon) to axially advance or retract components of the system 200, such as the outer shaft 202 or any other component passing therethrough, including a tube 260.

In some examples, the tube 260 is a torque transferring tube, configured to be movable rotatably relative to a central longitudinal axis C_{A} thereof (indicated, for example, in FIG. 5A), and/or rotatable relative to another shaft of the system 200, such as relative to the outer shaft 202. The handle 204 includes a rotation control assembly 210 (indicated, for example, in FIG. 5A-5B) which can be optionally operable by a knob of the handle 204, such as the illustrated rotatable knob 206a. A proximal portion of the tube 260 can extend through and configured to be engaged by the rotation control assembly 210, such that rotation of the assembly 210, either manually by rotating the knob 206a or via a motorized mechanism that can be operatively coupled to the assembly 210, allows the operator (such as a clinician) to effect rotation of tube 260. The terms "rotation control assembly 210" and "assembly 210", as used herein, are interchangeable.

The handle 204 can include a steering mechanism configured to adjust the curvature of the distal end portion of the system 200. In the illustrated example, the handle 204 can include an adjustment member, such as the illustrated rotatable knob 206b, which in turn is operatively coupled to the proximal end portion of a pull wire. The pull wire can extend distally from the handle 204 through the outer shaft 202 and has a distal end portion affixed to the outer shaft 202 at or near the distal end of the outer shaft 202. Rotating the knob 206b can increase or decrease the tension in the pull wire, thereby adjusting the curvature of the distal end portion of the system 200. Further details on steering or flex mechanisms for the system can be found in U.S. Patent No. 9,339,384, which is incorporated by reference herein.

The handle 204 can include additional adjustment mechanisms controllable by additional knobs to maneuver additional components of the system 200, such as axial movement of a perforating member 310 and/or axial movement of a tube 260 when provided as a separate tubular component, relative to other shafts of the system 200, as will be elaborated in greater detail below.

FIG. 5A is a perspective sectional view of an exemplary rotation control assembly 210. FIG. 5B is a perspective exploded view of the assembly 210 of FIG. 5A. FIG. 6 is a perspective view of a proximal portion of an exemplary tube 260 that can be engaged by the assembly 210 of FIGS. 5A-5B. FIG. 7 is a perspective sectional view of a body portion 222 of the assembly 210 of FIGS. 5A-5B, shown with transparency for illustrative purpose. FIG. 8 is a perspective view of the assembly 210 of FIGS. 5A-5B, with a rotatable body 212 thereof shown with transparency for illustrative purpose. FIG. 9A is a cross-sectional view of the assembly 210 of FIGS. 5A-5B. FIG. 9B is an enlarged view of region 9B of FIG. 9A. FIG. 10A is a perspective sectional view of the assembly 210 of FIGS. 5A-5B, which only one lug thereof shown for illustrative purpose. FIG. 10B is an enlarged view of region 10B of FIG. 10A. FIGS. 5A-10B are described herein together.

Rotation control assembly 210 can be housed inside the handle 204, and includes a rotatable body 212 defining an aperture 218 configured to allow passage of a tube, at least one first protrusion 238 having a first contact surface 240, and at least one second protrusion 242 having a second contact surface 244, wherein the first contact surface 240 and the second contact surface 244 face circumferentially opposite directions. The aperture 218 is sized to accommodate a proximal portion of a tube 260 therein. The tube 260 has a tube wall 262 defining a tube lumen 264, extending along a central longitudinal axis C_{A} around which both the rotatable body 212 and the tube 260 are configured to rotate. When the rotatable body 212 is disposed around the tube 260, the central longitudinal axis C_{A} of the tube 260 also serves as the central longitudinal axis of the rotatable body 212 and its aperture218.

The rotatable body 212 has an outer surface 214 oriented to face outwardly in the radial direction, and an inner surface 220 surrounding the aperture 218 and oriented to face inwardly in the radial direction, towards the central longitudinal axis C_{A}. The first radial protrusion 238 and the second radial protrusion 242 extend radially inwards from the inner surface 220, towards the central longitudinal axis C_{A}. Thus, the first protrusion 238 and the second protrusion 242 are configured to extend radially into the aperture 218. The terms "rotatable body 212" and "body 212", as used herein, are interchangeable. Any reference to a first protrusion 238 and/or a first contact surface thereof 240, and any reference to a second protrusion 242 and/or second contact surface 244 thereof, in a singular form throughout the specification, for an exemplary assembly 210 that can include a plurality of first protrusions 238, a plurality of first contact surfaces 240, a plurality of second protrusions 242, and/or a plurality of second contact surfaces 244, respectively, can refer to any one of the plurality of such protrusions and/or contact surfaces, unless stated otherwise.

In some examples, the assembly 210 can include lugs that form the radial protrusions. For example, an assembly 210 can include at least one first lug 246 and at least one second lug 252. The rotatable body 212 can have at least one first lug bore 230 configured to accommodate at least part of the corresponding first lug 246 therein, and at least one second lug bore 234 configured to accommodate at least part of the corresponding second lug 252 therein. Any reference to a first lug 246 or any portion thereof, to a first lug bore 230 or any portion thereof, to a second lug 252 or any portion thereof, or to a second lug bore 234 or any portion thereof, in a singular form throughout the specification, for an exemplary assembly 210 that can include a plurality of first lugs 246, a plurality of first lug bores 230, a plurality of second lugs 252, and/or a plurality of second lug bores 234, respectively, can refer to any one of the plurality of such lugs, lug bores, and/or portions thereof, unless stated otherwise.

The first and second lugs 246, 252 can be embedded in and extend radially inward from the respective first and second lug bores 230, 234. For example, each first lug bore 230 can define a lug bore opening 232 at the inner surface 220 through which the first lug 246 can extend, wherein the portion of the first lug 246 extending out of the lug bore opening 232 and into the aperture218 defines the first radial protrusion 238. Similarly, each second lug bore 234 can define a lug bore opening 236 at the inner surface 220 through which the second lug 252 can extend, wherein the portion of the second lug 252 extending out of the lug bore opening 236 and into the aperture218 defines the second radial protrusion 242.

In some examples, the rotatable body 212 can also define a knob 206 that can be exposed to a user of the handle 204, wherein the outer surface 214 can include, at least in part, an outer surface of the knob. In some examples, the knob 206 can be a separate component attached to the rotatable body 212. In some examples, as illustrated in FIG. 5A-5B, the knob 206 can include one or more torque keys 208 in the form of protrusions extending radially inwards, and the rotatable body 212 can include one or more torque slots 216 in the form of recesses defined over the outer surface 214, wherein the torque slots 216 are configured to receive the torque keys 208 such that rotational movement applied to the knob 206 causes rotation of the rotatable body 212 therewith, in the same direction of rotation.

Exemplary torque keys 208 and torque slots 216 are illustrated in FIGS. 5A-5B, but are removed from view in FIGS. 6-10A for illustrative purpose. It is to be understood that any other method of coupling a knob 206 to rotatable body 212 can be implemented. For example, the rotatable body 212 can include torque keys protruding radially outward from its outer surface 214, and the knob 206 can include corresponding torque slots. In some examples, fasteners can be used to couple the knob 206 to rotatable body 212. In some examples, the outer surface 214 of the rotatable body 212 can be non-circular, such as by including at lead one flat surface, and the knob 206 can include a complementary non-circular inner surface, forcing both to move rotationally in unison.

The tube 260 includes at least one first window 266 and at least one second window 270 extending through the thickness of the tube wall 262. Each first window 266 has a first contact edge 268 configured to be engaged by a corresponding first contact surface 240 of the first protrusion 238, and each second window 270 has a second contact edge 272 configured to be engaged by a corresponding second contact surface 244 of the second protrusion 242. Any reference to a first window 266 or a first contact edge 268 thereof, and/or to a second window 270 or a second contact edge 272 thereof, in a singular form throughout the specification, for an exemplary tube 260 that can include a plurality of first windows 266 and/or a plurality of second windows 270, respectively, can refer to any one of the plurality of such windows and/or contact edges thereof, unless stated otherwise.

The first contact surface 240 faces a first circumferential direction, while the second contact surface 244 faces a second circumferential direction, which is opposite to the first circumferential direction. For example, the first circumferential direction can be a clockwise direction 92, while the second circumferential direction can be a counterclockwise direction 94. When the first and second protrusions 238, 242 are provided as portions of first and second lugs 246, 252, the first lug 246 can have a clockwise facing sidewall 248 and a counterclockwise facing sidewall 250, wherein the portion of the clockwise facing sidewall 248 exposed out of the first lug bore 230 is the first contact surface 240. Similarly, the second lug 252 can have a counterclockwise facing sidewall 254 and a clockwise facing sidewall 256, wherein the portion of the counterclockwise facing sidewall 254 exposed out of the second lug bore 234 is the second contact surface 244.

The counterclockwise facing sidewall 250 of the first lug 246 can remain concealed inside the first lug bore 230 without extending into the first window 266 and/or contacting an edge of the window 266 which is opposite to the first contact edge 268. Similarly, the clockwise facing sidewall 256 of the second lung 252 can remain concealed inside the second lug bore 234 without extending into the second window 270 and/or contacting an edge of the window 270 which is opposite to the second contact edge 272.

The first and second protrusions 238, 242 extend into the first and second windows 266, 270, such that the first and second contact surfaces 240, 244 face the corresponding first and second contact edges 268. The first protrusion 238 includes front and rear axially facing surfaces 241 which are perpendicular to the first contact surface 240, and are configured to engage corresponding front and rear axially facing edges 268 of the first window 266. Similarly, the second protrusion 242 includes front and rear axially facing surfaces 245 which are perpendicular to the second contact surface 244, and are configured to engage corresponding front and rear axially facing edges 273 of the second window 270. The axial dimension of the first window 266 defined between its axially facing edges 269, and the axial dimension of the second window 270 defined between its axially facing edges 273, can be similar to the respective axial dimension of the first protrusion 238 defines between its axially facing surfaces 241, and the axial dimension of the second protrusion 242 defines between its axially facing surfaces 245, to prevent unintentional axial displacement of the rotatable body 212 relative to the tube 260.

In use, when the body 212 is rotated in a first circumferential direction, such as a clockwise direction 92, optionally by manipulation the knob 206, the first contact surface 240 is pushed against the corresponding first contact edge 268, forcing the tube 260 to rotate along with the body 212 in the same direction. The second contact surface 244 can be either in contact with, or circumferentially spaced from, the corresponding second contact edge 272, during rotation of the body 212 in the first direction. When the body 212 is rotated in the second direction, such as a counterclockwise direction 94, the second contact surface 244 is pushed against the corresponding second contact edge 272, forcing the tube 260 to rotate along with the body 212 in the same direction. The first contact surface 240 can be either in contact with, or circumferentially spaced from, the corresponding first contact edge 268, during rotation of the body 212 in the second direction.

The rotatable body 212 can be substantially cylindrical, through other shapes are contemplated, including non-circular cross-sectional shapes. In some examples, the rotatable body 212 can be formed of two or more separate components attachable to each other, such as two body portions 222 shown in the example illustrated in FIGS. 5A-10B. An optional advantage of forming the rotatable body 212 from two separate components, as opposed to forming a unitary component, can be that each body portion 222 can be formed via molding. Moreover, the modular design can allow for easier assembly, such as allowing convenient insertion of first and second protrusions 238, 242 into corresponding windows 266, 270 of the tube 260 prior to securing both body portions 222 to each other around the tube 260.

The body portions 222 are configured to be secured to each other, optionally in a reversible manner that will allow both portions 222 to be disconnected, for example when maintenance is required or replacement of some components of the system 200 is desired. In some examples, fasteners or connectors can be used to attach both body portions to each other. In some examples, the connectors can be in the form of bolts or screws 276 that can be connected, such as by being screwed into, corresponding nuts of insert 278. In some examples, the body portions 222 can include connector bored 224 that can be exposed via connector insertion opening 226 at the outer surfaces 214, and counterbores 228 aligned with the connector bores.

In some examples, the connector comprises a threaded screw 276 configured to mate with an insert 278, which can be a heat-set threaded insert. In some examples, the threaded screw 276 can be an M2 screw, and the insert 278 can be an M3 heat-set threaded insert. The heat set threaded insert can be installed in the counterbore, and a screw 276 can be then inserted through the connector insertion opening 226, and threaded into the insert 278. It is to be understood that the body portions 222 can be attached to each other in any other suitable manner, such as by one body portion 222 including pins or bosses that can be inserted and optionally snap-fit in complementary openings or bores of the other body portion 222, such as by both body portions 222 being screwed to each other, such as by both body portions 222 being attached to each other by interference fit, and the like.

The tube 260 can be an elongated shaft or tube, such as a torque-transferring shaft configured to rotate along its entire length, when rotated at the handle 204 by assembly 210. In sone example, the tube 260 can be a flexible tube, configured to bend when passed through curved portions of the patient's vasculature, for example. In some examples, the tube 260 can comprise a hypotube, having series of slits along at least part of a length thereof. In some examples, the tube lumen 264 is sized to allow passage of an inner shaft 274 therethrough. The inner shaft 274 can coaxially extend through the tube 260, and can be axially movable relative to the tube 260. The inner shaft 274 can be devoid of windows or openings formed through the thickness of its wall at the regions aligned with windows 266 or 270, such that the first and second protrusions 242 can extend into the first and second windows 266, 270, but terminate prior to reaching the outer surface of the inner shaft 274.

As mentioned above, the tube can be a thin-walled tube 260. The terms "thin-walled tube 260" and "tube 260", as used herein, are interchangeable. Conventional rotation control assemblies, configured to facilitate rotational movement of regularly sized or thick-walled tubes, can include, in some cases, a single protrusion extending radially inwards into a receiving bore or opening of the tube, such that one sidewall of the single protrusion can face and be in contact with one edge of the tube's opening, and the opposite sidewall of the protrusion can face and be in contact with the opposite edge of the opening. In such cases, rotation in either direction will cause the appropriate sidewall of the protrusion to press against the corresponding edge of the tube's opening, allowing for bi-directional rotational movement of the tube, facilitated by a single protrusion. Though more than one protrusion can be provided in such cases, a single protrusion may be sufficient to rotate the tube at least to some extent.

In contrast to such conventional configurations, a thin-walled tube 260 has a wall thickness that will not allow utilization of a single protrusion into an opening or window of the tube, since the relatively thin wall 262 will not allow both edges of such an opening to be contacted by both sidewalls of a single protrusion. As shown throughout FIGS. 5A-10B, a first contact edge 268 of the first window 266 can be contacted by the first contact surface 240, while the opposite edge of the window 266 remains free or engagement with any other portion of the first protrusion 238, and a second contact edge 272 of the second window 270 can be contacted by the second contact surface 244, while the opposite edge of the window 270 remains free or engagement with any other portion of the second protrusion 242.

Thus, in order to allow for bi-directional rotational movement of a thin-walled tube 260, at least two contact surfaces facing opposite circumferential directions need to be provided, each configured to engage with a different corresponding contact edge of the tube 260. It may be further noted that a single protrusion could have been utilized, theoretically, to provide for bi-directional rotational movement of a thin-walled tube 260, if allowed to extend radially inward past a window formed in the tube 260 and into the tube lumen 264 to a depth sufficient to allow both sidewalls thereof to contact both circumferential edges of the window. However, when the tube 260 is designed to allow passage of another inner shaft 274 through its lumen 264, extension of a radial protrusion past the thickness of the tube wall 262 may not be allowed.

The tube wall 262 has a wall thickness T_{W}, defined as the difference between the tube outer diameter D_{O} and the tube inner diameter D_{I}. The aperture218 defines an aperture diameter D_{C} that can be substantially equal to, or slightly greater than the tube outer diameter D_{O}. For example, a small radial gap G_{R} can exist between the inner surface 220 and the tube wall 262. The radial gap G_{R}, defined as (D_{C}- D_{O})/2, can be, in some examples, less than 15% of the aperture diameter D_{C}, less than 10% of the aperture diameter D_{C}, or less than 5% of the aperture diameter D_{C}.

Each contact surface 240, 244 extends radially past the inner surface 220 along a contact surface radial length L_{R}. In some examples, the contact surface radial length L_{R} is equal to the wall thickness T_{W}. In some examples, such as when the aperture diameter D_{C} is slightly greater than the tube outer diameter D_{O}, the contact surface radial length L_{R} can be slightly greater than the wall thickness T_{W}. For example, the contact surface radial length L_{R} can be equal to or less than the sum of the radial gap G_{R} and wall thickness T_{W} [L_{R} ≤ (G_{R}+T_{W})]. In this manner, contact between the contact surface and corresponding contact edge can be maintained during rotational movement is the appropriate direction, without the radial protrusion extending past the corresponding window, allowing for undisturbed passage of an inner shaft 274 through the tube lumen 264.

A thin-walled tube 260 is defined as a tube having a wall thickness T_{W} which is too thin to allow for adequate surface area contact between both circumferential sidewalls of a protrusion extending into an opening formed in the tube's wall, with both corresponding circumferential edges of the opening, without allowing extension of a protrusion into the lumen defined by the thin-walled tube. In some examples, the wall thickness T_{W} is not greater than about 200 micrometers (µ). In some examples, the wall thickness T_{W} is in a range between about 50µ and about 200µ, inclusive. In some examples, the wall thickness T_{W} is less than about 15% of the tube outer diameter D_{O}. In some examples, the wall thickness T_{W} is less than about 10% of the tube outer diameter D_{O}. In some examples, the wall thickness T_{W} is less than about 8% of the tube outer diameter D_{O}. In some examples, the wall thickness T_{W} is less than about 15% of the aperture diameter D_{C}. In some examples, the wall thickness T_{W} is less than about 10% of the aperture diameter D_{C}. In some examples, the wall thickness T_{W} is less than about 8% of the aperture diameter D_{C}.

In some examples, the maximal contact surface radial length L_{R}, measured from the inner surface 220 to a tip of the corresponding protrusion 238 or 242, is not greater than about 200µ. In some examples, the contact surface radial length L_{R} is not greater than about 300µ. In some examples, the contact surface radial length L_{R} is not greater than about 200µ. In some examples, the contact surface radial length L_{R} is less than about 30% of the aperture diameter D_{C}. In some examples, the contact surface radial length L_{R} is less than about 20% of the aperture diameter D_{C}. In some examples, the contact surface radial length L_{R} is less than about 15% of the aperture diameter D_{C}. In some examples, the contact surface radial length L_{R} is less than about 10% of the aperture diameter D_{C}. The terms "contact surface radial length L_{R}" and "maximal contact surface radial length L_{R}", as used herein, are interchangeable.

Various exemplary implementations for systems 200 and/or assemblies 210 thereof can be referred to, throughout the specification, with superscripts, for ease of explanation of features that refer to such exemplary implementations. It is to be understood, however, that any reference to structural or functional features of any system, assembly or component, without a superscript, refers to these features being commonly shared by all specific exemplary implementations that can be also indicated by superscripts. In contrast, features emphasized with respect to an exemplary implementation of any device, system or component, referred to with a superscript, may be optionally shared by some but not necessarily all other exemplary implementations. For example, rotation control assembly 210^{a} illustrated in FIGS. 5A-10B is an exemplary implementation of rotation control assembly 210, and thus can include any of the features described for rotation control assembly 210 throughout the current disclosure, except that while a assembly 210 can have any number of first and second protrusions 238, 242, rotation control assembly 210^{a} is shown to include two first protrusions 238 and two second protrusions 242.

In some examples, two or more first protrusions 238 can be axially aligned with each other, but circumferentially offset from one another. In the illustrated examples, two first protrusions 238a and 238b extend from radially opposing sides of the inner surface 220, though other spatial arrangements can be implemented. Similarly, in some examples, two or more second protrusions 242 can be axially aligned with each other, but circumferentially offset from one another. In the illustrated examples, two second protrusions 242a and 242b extend from radially opposing sides of the inner surface 220, though other spatial arrangements can be implemented.

In some examples, the first protrusions 238 and the second protrusions 242 can be axially offset from each other. In some examples, a first contact surface 240 is circumferentially aligned, and is axially offset, relative to a corresponding second contact surface 244. In the example illustrated in FIG. 9, first contact surface 240a and second contact surface 244a are axially spaced from each other, but are circumferentially aligned with each other such that both are positioned at, or in close proximity to, the same circumferential position along the circumference of the aperture218. Similarly, first contact surface 240b and second contact surface 244b are illustrated in the example shown in FIG. 9 to be axially spaced from each other, but circumferentially aligned with each other.

The arrangement of first and second windows 266, 270 can generally match the first and second protrusions 238, 242. An exemplary tube 260^{a} designed for use with rotation control assembly 210^{a} can include two first windows 266 and two second windows 270. In some examples, two or more first windows 266 can be axially aligned with each other, but circumferentially offset from one another. In the illustrated examples, two first windows 266a and 266b are formed along radially opposing sides of the tube wall 262, though other spatial arrangements can be implemented. Similarly, in some examples, two or more second windows 270 can be axially aligned with each other, but circumferentially offset from one another. In the illustrated examples, two second windows 270a and 270b are formed along radially opposing sides of the tube wall 262, though other spatial arrangements can be implemented.

In some examples, the first windows 266 and the second windows 270 can be axially offset from each other. In some examples, a first contact edge 268 is circumferentially aligned, and is axially offset, relative to a corresponding second contact edge 272. In the example illustrated in FIG. 6, first contact edge 268a and second contact edge 272a are axially spaced from each other, but are circumferentially aligned with each other such that both are positioned at, or in close proximity to, the same circumferential position along the circumference of the tube 260. Similarly, first contact edge 268b and second contact edge 272b are illustrated in the example shown in FIG. 6 to be axially spaced from each other, but circumferentially aligned with each other.

It is to be understood that while two first protrusions 238 extending into two first windows 266, and two second protrusions 242 extending into two second windows 270, are shown in FIGS. 5A-10B, this is shown by way of illustration and not limitation, and that an assembly 210 can include more than two first protrusions 238 that can extend into more than two first windows 266, and more than two second protrusions 242 that can extend into more than two second windows 270.

FIG. 11 is a perspective view of an exemplary rotation control assembly 210^{b}, shown with partial transparency for illustrative purpose. Rotation control assembly 210^{b} is an exemplary implementation of rotation control assembly 210, and thus can include any of the features described for rotation control assembly 210 throughout the current disclosure, except that the rotation control assembly 210^{b} comprises a single first protrusion 238 and a single second protrusion 242, which can extend into a single first window 266^{b} and a single second window 270^{b}, respectively, of an exemplary tube 260^{b}shown in FIG. 12.

In the example illustrated in FIG. 11, the single first contact surface 240 and the single second contact surface 244 are circumferentially aligned. In cases of circumferential alignment between a first contact surface 240 and a second contact surface 244, both contact surfaces 240, 244 will be axially spaced from each other, as is the case shown for both examples of assemblies 210^{a} and 210^{b}. Similarly, in the example illustrated in FIG. 12, the single first contact edge 268 and the single second contact edge 272 are circumferentially aligned. In cases of circumferential alignment between a first contact edge 268 and a second contact edge 272, both contact edges 268, 272 will be axially spaced from each other, as is the case shown for both examples of tubes 260^{a} and 260^{b}.

While both body portions 222 illustrated in FIGS. 5A-10B for exemplary assembly 210^{a} can be generally similar to each other, each including, for example, one first lug bore 230 and one second lug bore 234, the body portion 222 illustrated in FIG. 11 for exemplary assembly 210^{b} are different in that one of the body portions 222a can include a single first lug bore 230a and a single second lung bore 234, while the other body portion 222b can be devoid of lug bores.

The term "lug bore", as used herein without a "first" or "second" prefix, refers to any of the first and second log bores 230 and 234. The terms "radial protrusion" and "protrusion", as used herein with respect to any of a first protrusion 238 and/or a second protrusion 242, are interchangeable. The terms "protrusion" or "radial protrusion", as used herein without a "first" or "second" prefix, refer to any of the first and second protrusions 238 and 242. The term "contact surface", as used herein without a "first" or "second" prefix, refers to any of the first and second contact surfaces 240 and 244. The term "window", as used herein without a "first" or "second" prefix, refers to any of the first and second windows 266 and 270. The term "contact edge", as used herein without a "first" or "second" prefix, refers to any of the first and second contact edges 268 and 272.

FIG. 13 is a perspective view of an exemplary rotation control assembly 210^{c}, shown with partial transparency for illustrative purpose. Rotation control assembly 210^{c} can be generally similar to assembly 210^{b}, and comprise a single first protrusion 238 and a single second protrusion 242, which can extend into a single first window 266^{c} and a single second window 270^{c}, respectively, of an exemplary tube 260^{c} shown in FIG. 14, except that the first contact surface 240 of assembly 210^{c} can be both circumferentially offset and axially offset from the second contact surface 244. For example, the first and second protrusions 238 and 242 of assembly 210^{c} can be radially opposed relative to each other, though other circumferential positions thereof are contemplated. In the example illustrated in FIG. 14, the single first contact edge 268 and the single second contact edge 272 are also circumferentially offset and axially offset from each other.

FIG. 15 is a perspective view of an exemplary rotation control assembly 210^{d}, shown with partial transparency for illustrative purpose. Rotation control assembly 210^{d} can be generally similar to assembly 210^{c}, and comprise a single first protrusion 238 and a single second protrusion 242, which can extend into a single first window 266^{c} and a single second window 270^{c}, respectively, of an exemplary tube 260^{c} shown in FIG. 14, except that whole the first contact surface 240 of assembly 210^{d} is circumferentially offset from the second contact surface 244 in a similar manner to that described with respect to assembly 210^{c}, the first protrusion 238 of assembly 210^{d} is axially aligned with the second protrusion 242. For example, the first and second protrusions 238 and 242 of assembly 210^{d} can be radially opposed relative to each other, though other circumferential positions thereof are contemplated.

In contrast to examples in which first and second contact surfaces 240, 244 are circumferentially aligned, when the first and second contact surfaces 240 and 244 are circumferentially offset from each other, they can be either axially aligned with each other as illustrated in FIG. 15, or axially offset from each other as shown in FIG. 13.

In the example illustrated in FIG. 16, the single first contact edge 268 and the single second contact edge 272 are also shown to be circumferentially offset from each other but axially aligned with each other. A circumferential offset arrangement between first and second windows 266 and 270 similarly allows first and second contact edges 268 and 272 to be axially aligned with each other as illustrated in FIG. 16, or axially offset from each other as shown in FIG. 14.

An advantage of the example illustrated for assembly 210^{d} in FIG. 15, over that of assembly 210^{b} for example, is that both body portions 222 of assembly 210^{d} can be similarly shaped, each including a similarly-formed lug bore, wherein the lug bore of one body portion 222a can serve as a first lug bore 230, while a lug bore of the other body portion 222b can serve as a second lug bore 234.

FIG. 17A is a perspective sectional view of a portion of an exemplary rotation control assembly 210^{e}, and FIG. 17B shows an enlarged view of region 17B of FIG. 17A. The portion of assembly 210^{e} shown in FIG. 17A can be either representative of a single body portion 222 of a rotatable body 212^{e} formed by two attachable body portion 222, or of a selected portion of a unitary body 212^{e}. Rotation control assembly 210^{e} is an exemplary implementation of rotation control assembly 210, and thus can include any of the features described for rotation control assembly 210 throughout the current disclosure, except that the rotation control assembly 210^{e} comprises protrusions which are integrally formed with the rotatable body 212. The rotatable body 212^{e} or a portion thereof, such as a body portion 222, together with any first protrusion 238^{e} and/or second protrusion 242^{e} extending from an inner surface 220 thereof, form a unitary body that can be optionally molded or otherwise formed as a single piece material, without including lug bores or lugs embedded therein.

A second protrusion 242^{e} is shown in FIG. 17A-17B as an integrally-formed protrusion by way of illustration and not limitation, and it is to be understood that a first protrusion 238^{e} can be similarly integrally formed with the body 212^{e}. It is to be understood that any exemplary assembly 210 described above, including assemblies 210^{a}, 210^{b}, 210^{c} or 210^{d} can have lugs embedded in the body 212, the lugs defining the protrusions at end portions thereof, or can have protrusions which are integrally formed with the rotatable body 212, as described with respect to assembly 210^{e} shown in FIG. 17A-17B.

In some examples, the first lug 246 and the second lug 252 are formed from a different material than that of the rotatable body 212. In some examples, the first lug 246 and second lug 252 are stiffer than the rotatable body 212. In some examples, the rotatable body 212 and/or body portions 222 thereof can be formed as molded plastic components. When formed as a molded plastic component, a body 212 or body portion 222 thereof, that includes one or more integrally formed protrusions, means that the protrusions are also made of plastic material. In some cases, the forces applied on the relatively limited contact area offered by the protrusions may degrade the protrusions over time if formed of polymeric materials, such as molded plastic. For example, in some implementations of a thin-walled tube 260 engaged by the protrusions, the tube 260 need to withstand axial forces of about 100N and/or torque in a range between about 1and about 10Nm. In such cases, it may be preferable to form the protrusion from a more resistant and durable material, such as metal, in which case metallic lugs can be embedded in plastic-molded body 212, the metallic lugs defining, at end portions thereof, metallic protrusions that are more durable.

In some examples, body 212 and/or body portions 222 thereof can be overmolded on first and second lugs 246, 252 that can be made of a material such as stainless steel, aluminum, nitinol or other suitable materials. Overmolding components such as body portions 222 is inexpensive and avoids difficult to validate adhesive bonding steps during manufacture.

FIGS. 18A-18H illustrate some steps in a method for utilizing a system 200^{f} for forming an opening within a target tissue. An exemplary implementation of the method is illustrated in FIGS. 18A-18H with respect to forming a leaflet hole inside a host leaflet, which can be performed prior to implanting a guest prosthetic valve inside the host valvular structure. The system 200^{f} can be used to perforate a host leaflet 10, such as a native leaflet 30 or a prosthetic valve leaflet 114 of a previously implanted prosthetic valve. System 200^{f} can include any exemplary assembly 210 and tube 260 disclosed herein, such as any of the exemplary rotation control assemblies 210^{a}, 210^{b}, 210^{c}, 210^{d} and/or 210^{e}, and any of the exemplary tubes 260^{a}, 260^{b}, 260^{c} or 260^{d}. System 200^{f}, an example of which is also illustrated in FIG. 4, can further include a perforating member 310 extending through the outer shaft 202, wherein the perforating member 310 is be axially movable through a lumen 203 of the outer shaft 202, relative to the outer shaft 202. The system 200^{f} can be utilized in a method that includes steps of positioning an expansion member 320, such as an inflatable balloon (shown in FIG. 18D-18F), inside a puncture formed by the perforating member 310, for expanding the puncture and forming a wider opening 52 in the host leaflet 10. The expansion member 320 can be optionally advanced through the outer shaft lumen 203, or a different shaft or catheter can be used for delivery the expansion member.

The perforating member 310 can define a perforating member lumen 312 through which a guidewire 80 can extend. The perforating member 310 can comprise a perforating member distal end portion 314 terminating at a perforating member tip 318, configured to pierce a target tissue such as a host leaflet 10 of a host valvular structure 12 to form a pilot puncture 50 in the host leaflet 10, when a perforating member distal end portion 314 is positioned distal to the outer catheter 202.

In some examples, the perforating member 310 may include and/or be a needle, such as a spring-loaded needle and/or a Veress needle. In the example illustrated in FIGS. 18A-18C, the perforating member is a hollow needle 310 that defines a needle lumen 312, and the perforating member distal end portion is a needle distal end portion 314 that can define an angled surface 316 terminating at a needle tip 318. In some examples, at least a portion of the needle 310 is formed as a hypotube, configured to increase flexibility thereof.

In some examples, the needle 310 of a system 200^{f} can be biased, in a free state thereof, to the bent state, and the system 200^{f} can further comprise an overtube 342 defining an overtube lumen 344, and the needle 310 can be axially movable through the overtube lumen 344, relative to the overtube 342. The overtube 342 can be axially movable through, and relative to, the outer shaft 202.

The distal end portion of the system 200^{f} is configured to be inserted into a patient’s vasculature, such as within an ascending aorta, and to be advanced towards the host leaflet 10. Positioning the system 200^{f} relative to the host leaflet 10 may comprise advancing the system 200^{f} toward the leaflet over a guidewire 80. The needle lumen 312 can be configured to accommodate a guidewire 80 that can be passed therethrough. In such examples, the guidewire 80 can be inserted into the patient’s vasculature, and then the needle 310 and/or other shafts or tubes of the system 200^{f} may be advanced toward the host leaflet 10 over the guidewire 80.

During delivery, the needle distal end portion 314 can be retained inside the overtube lumen 344, retaining the sharp needle tip 318 therein as illustrated in FIG. 18A. This position conceals the needle tip 318 from the surrounding anatomy, to protect the anatomical structures from being engaged or punctured by the needle tip 318 during advancement towards the site of treatment.

During delivery, the overtube 342 can be mostly or entirely retained inside the outer shaft 202. Upon approximation to the host valvular structure 12, at least part of the overtube 342 can be advanced out of the outer shaft 202 towards the host leaflet 10, but without extending all the way to the host leaflet 10. The diameter of the overtube lumen 344 can be substantially similar to the outer diameter of the needle 310. The overtube 342 can be flexible enough to passively bend inside the outer shaft 202 while the outer shaft 202 is bent through curved portions of the patient's vasculature, or when the outer shaft 202 is articulated if provided as a steerable catheter. Such passive bending of the overtube 342 will cause the flexible needle 310 to passively bend therewith. The needle 310 can be then axially translated in a distal direction towards the leaflet 10, exposing a distal portion thereof out of the overtube lumen 344.

In some examples, at least one catheter or shaft of the system 200, such as the outer shaft 202, can be steerable so as to navigate the distal portion of the system 200 toward the desired host leaflet 10, such as a leaflet that can be closer to the left coronary ostium. In some cases, orienting a distal end portion of the system 200 sideways, towards a host leaflet 10, optionally in some proximity to the nadir of the leaflet, can orient the distal end of the outer shaft 202 towards a host interior surface 14, which can be the interior surface of the aortic wall if the host valve is the native valve, or an interior surface of a frame of a previously implanted prosthetic valve serving as the host valve. In such cases, merely advancing a needle in the distal direction to expose it prior to penetrating through the host leaflet 10, can direct the needle towards the host interior surface 14 instead of the host leaflet 10 itself.

As mentioned above, the needle 310 of system 200^{f}can be biased, in a free state thereof, to assume a bent state, such as by being formed of a shape memory material (for example, Nitinol) which is shape-set to the bent configuration. Thus, as soon as the needle 310 is exposed out of the overtube 342, the exposed portion, which is no longer bound by the inner surface of the overtube 342, is free to assume its bent shape, advantageously orienting it toward the host leaflet 10 as illustrated in FIG. 18B, such that during advancement of the needle 310, its distal portion 314 can contact and pierce through the host leaflet 10, without posing a risk of contacting and damaging adjacent anatomical structures, such as the host interior surface 14. As shown in FIG. 18C, further advancement of the needle 310 in its exposed bent state will cause it to puncture the host leaflet 10 to form a pilot puncture 50 within host leaflet 10.

In the case of a pre-shaped needle 310 configured to assume a bent shape in a free state thereof, the needle 310 will bend in a specific predefined orientation, which may be different than the orientation desired at the site of implantation. For example, when the needle 310 is exposed out of overtube 342, it may bend in any direction and not necessarily in the direction oriented towards the desired host leaflet 10. In some examples, the tube 260 is the needle 310, such as a pre-shaped needle 310 of system 200^{f}, meaning that the proximal portion of the needle 310 extending into the handle 204 can be engaged and rotated by the rotation control assembly 210. Thus, a control rotation assembly 210 can be operated (such as by rotating a knob 206 associated therewith) to rotate the needle 310 in a desired direction, angularly orienting the needle's distal end portion 314 towards the host leaflet 10. When the needle 310 is the tube 260, the guidewire 80 extending through the needle lumen 312 can be the inner shaft 274.

In some examples of the method, once a portion of the needle 310 is positioned past the host leaflet 10, the guidewire 80 can be advanced through the needle lumen 312 to terminate with guidewire tip 82 at a position distal to the pilot puncture 50 of host leaflet 10. Subsequent to forming the pilot puncture 50 and optionally advancing the guidewire 80 to extend therethrough, the needle 310 can be optionally retracted, leaving the guidewire 80 extending through the pilot puncture 50.

In some examples, the guidewire 80 can be advanced to terminate distal to the host leaflet 10 after formation of the pilot puncture 50 by the needle 310. In some examples, the guidewire 80 can be advanced simultaneously with advancement of the needle 310 towards the host leaflet 10 and/or during formation of the pilot puncture 50.

As mentioned above, the method can further include steps of positioning an expansion member 320 inside the pilot puncture 50. An expansion member can be either part of the system 200, or provided as a separate component advanced into a pilot puncture formed by a perforating member 310 of the system 200. The expansion member 320 may include and/or be any suitable structure for expanding the pilot puncture 50 to form a leaflet opening 52. In some examples, the expansion member 320 may have a circular profile when in the radially expanded configuration. This is not required of all examples, however, and it additionally is within the scope of the present disclosure that the expansion member 320 may have a non-circular profile when in the radially expanded configuration.

In some examples, the expansion member 320 is an inflatable hole-dilating balloon 322 that can be mounted on a distal portion of a balloon catheter 324. In some examples, a balloon catheter 324 carrying hole-dilating balloon 322 can be advanced over the guidewire 80 towards the pilot puncture 50 formed in host leaflet 10, after retraction of the perforating member 310, as shown in FIG. 18D. The hole-dilating balloon 322 is configured to transition between a radially deflated state and a radially inflated state. The balloon catheter 324 can define a balloon catheter lumen 326 (indicated, for example, in FIGS. 21A-21B), through which a guidewire 80, and one or more additional shafts of the system 200 (or another system that includes the hole-dilating balloon 322), can optionally extend. The balloon catheter 324 can extend through a handle 204 of the system 200 (or a handle of or another system that includes the hole-dilating balloon 322) and be fluidly connectable to a fluid source (not shown) for inflating the hole-dilating balloon 322. The fluid source comprises an inflation fluid. The term "inflation fluid", as used herein, means a fluid (for example, saline, though other liquids or gas can be used) used for inflating the balloon 322. The inflation fluid source is in fluid communication with the balloon catheter lumen 326, such that fluid from the fluid source can flow through the balloon catheter lumen 326 into balloon 322 to inflate it.

In some examples, a dilator 330 (see FIGS. 18D and 21A-21B) can be positioned distal to the hole-dilating balloon 322 (or other suitable expansion member 320). The dilator 330 can be either part of the system 200, or provided as a separate component advanced towards a pilot puncture formed by a perforating member 310 of the system 200. The dilator 330 can be conical or frustoconical in shape, and include a dilator tapering portion 334 terminating at a dilator distal end 332, and a dilator proximal portion 336 that can be coupled to a dilator shaft 338 that extends proximally therefrom. The dilator shaft 338 and dilator 330 collectively define a dilator lumen 340 open ended at the dilator distal end 332. Attachment of the dilator shaft 338 to the dilator proximal portion 336 can be achieved by a variety of methods, such as overmolding, radio-frequency welding, through an adhesive, and/or a combination thereof. In some examples (not illustrated), the dilator shaft 338 can extend through the entire length of the dilator 330, such that a distal end of the dilator shaft 338 is aligned with the dilator distal end 332. In some examples (not illustrated), the dilator shaft 338 is coupled to one or more components, such as collars or other connectors, which are in turn attached to the dilator 330.

In some examples, the hole-dilating balloon 322 is coupled to a distal end portion of the balloon catheter 324 at its proximal end, while the balloon's distal end can be coupled, directly or indirectly, to another component of the system, such as the dilator 330 or dilator shaft 338. In the examples illustrated in FIGS. 18D and 21A-21B, the hole-dilating balloon 322 is shown to be coupled to the dilator proximal portion 336. The dilator proximal portion 336 can optionally include an outer step configured to accommodate the distal end of the balloon 322, such that the outer surface of the balloon 322 can be flush or otherwise relatively continuous with the outer surface of the dilator 330.

In some examples, the balloon catheter 324 with hole-dilating balloon 322 and/or dilator 330 are advanced towards the pilot puncture 50 of host leaflet 10 over the same guidewire 80 used for advancement of the perforating member 310 towards the host leaflet 10 for formation of the pilot puncture 50. The balloon catheter 324 can extend through an outer shaft 302 which can be either the outer shaft 202 of system 200, or a different outer shaft. In some examples, the balloon catheter 324 with hole-dilating balloon 322 and/or dilator 330 are advanced towards the pilot puncture 50 of host leaflet 10 through the lumen 203 of the same outer shaft 202 used for advancement of the perforating member 310 therethrough towards the host leaflet 10 for formation of the pilot puncture 50. In some examples, the balloon catheter 324 with hole-dilating balloon 322 and/or dilator 330 are advanced towards the pilot puncture 50 of host leaflet 10 through the lumen of an outer shaft 302 that can be different from the outer shaft 202 of system 200, but may be similar in all respect to any example describe above for outer shaft 202, and in the interest of brevity will not be described further.

The outer shaft 302, balloon catheter 324, and/or dilator shaft 338, can be configured to be axially movable relative to each other. For example, a proximally oriented movement of the outer shaft 302 relative to the balloon catheter 324, or a distally oriented movement of the balloon catheter 324 relative to the outer shaft 302, can expose the hole-dilating balloon 322 from the outer shaft 302. Similarly, a proximally oriented movement of the dilator 330 relative to the outer shaft 302, or a distally oriented movement of the outer shaft 302 relative to the dilator 330, can expose the dilator 330 and axially translate it in a desired direction.

In some examples, such as when the hole-dilating balloon 322 is attached at both ends thereof to the dilator 330 and balloon catheter 324, both the dilator 330 with dilator shaft 338 and the balloon catheter 324 can be configured to move simultaneously in the axial direction, without necessarily being axially movable relative to each other, or while axial movement of one relative to the other is limited. In such examples, axial movement of the balloon catheter 324 is configured to cause movement of the dilator shaft 338 therewith, or axial movement of one of the dilator shaft 338 or dilator 330 is configured to cause movement of the balloon catheter 324 therewith.

As mentioned, the balloon catheter 324 with hole-dilating balloon 322 can be part of a system 200 that includes the perforating member 310. The proximal ends of various components the system, such as outer shaft 202, perforating member 310, balloon catheter 324, and/or dilator shaft 338, can be coupled to the handle 204. During delivery, the handle 204 can be maneuvered by an operator (for example, a clinician or a surgeon) to axially advance or retract components of the system, such as outer shaft 202, perforating member 310, balloon catheter 324, and/or dilator shaft 338, through the patient's vasculature and/or along the target site of treatment, and to expand an expansion member 320, such as to inflate a hole-dilating balloon 322 mounted on the balloon catheter 324 so as to enlarge a leaflet opening 52, as will be elaborated in further detail below, and to deflate the balloon 322 and optionally retract it.

Subsequent to forming the pilot puncture 50 and extending the guidewire 80 therethrough, and optionally after retraction of the perforating member 310, such as the needle 310 of a system 200^{f}, a hole-dilating balloon 322 carried over a balloon catheter 324 can be advanced towards the host leaflet 10, as shown in FIG. 18D.

When the outer shaft 302 through which expansion member 320 is passed, is the outer shaft 202 through which perforating member 310 extends, the needle 310 can be retracted through the outer shaft lumen 203 while the outer shaft 202 remains in position, in the vicinity of the host leaflet 10, with the guidewire 80 extending through the outer shaft lumen 203 into the pilot puncture 50. This allows the balloon catheter 324, and optionally dilator shaft 338, to be advanced towards the pilot puncture 50 of the host leaflet 10 over the guidewire 80, through the lumen 203 of the same outer shaft 202. In some examples, the outer shaft 202 can be retracted along with the needle 310, and then readvanced towards the host leaflet 10 with the balloon catheter 324 and/or dilator shaft 338 extending therethrough. When the outer shafts 302 and 202 are separate shafts, the outer shaft 202 can be retracted along with the needle 310, and the outer shaft 302 can be then advanced towards the host leaflet 10 with the balloon catheter 324 and/or dilator shaft 338 extending therethrough.

In some examples, when a dilator 330 is present distal to the expansion member 320 (such as balloon 322) as also shown in the example illustrated in FIG. 18D, the dilator 330 can be advanced, optionally along with the balloon catheter 324 and balloon 322, towards the host leaflet 10. In such examples, the dilator 330 can be inserted into the pilot puncture 50 to expand the pilot puncture 50, as shown in FIG. 18E. As the dilator 330 is inserted into the host leaflet 10, the inherent resiliency of the leaflet 10 may urge the leaflet 10 radially inwardly against the dilator 330. The dilator 330 can have sufficient stiffness to facilitate advancement thereof through the leaflet 10, wherein the gradually tapering shape of the dilator 330 facilitates expanding the pilot puncture 50 to a greater diameter.

In a subsequent step of the method, illustrated in FIG. 18F, the hole-dilating balloon 322 may be inserted within the pilot puncture 50, such as by further advancement of the dilator 330 with dilator shaft 338 and/or balloon catheter 324. With the hole-dilating balloon 322 received within the pilot puncture 50, inflating the balloon 322 to transition it from a radially deflated state (FIG. 18F) to a radially inflated state (FIG. 18G) can expand the pilot puncture 50 to form a leaflet opening 52 that can be sized to receive a guest prosthetic valve 100 in the radially compressed or crimped configuration. After the hole-dilating balloon 322 is inflated to form the leaflet opening 52 as shown in FIG. 18G, the balloon 322 is deflated, as shown in FIG. 18H, optionally allowing for insertion of a guest prosthetic valve inside the leaflet opening 52. Further examples of systems that include a needle for perforating a host leaflet, and methods for utilization thereof, can be found, for example, in International Application No. PCT/US2021/052745 and U.S. Provisional Application No. 63/589,104, all of which are incorporated herein by reference in their entireties.

In some examples, inflating the hole-dilating balloon 322 within the host leaflet 10 serves to increase a diameter of the pilot puncture 50 such that the resulting leaflet opening 52 is a hole with an increased diameter relative to the pilot puncture 50. In some examples in which the leaflet opening 52 is a hole, the leaflet opening 52 may be a substantially circular hole. In other examples, the leaflet opening 52 may be non-circular (for example, elliptical or asymmetric). In such examples, the diameter of the leaflet opening 52 may refer to any suitable dimension of the leaflet opening 52, such as a minimum diameter of the leaflet opening 52, a maximum diameter of the leaflet opening 52, and/or an average diameter of the leaflet opening 52.

In some examples, inflating the hole-dilating balloon 322 within the host leaflet 10 may cause the host leaflet 10 to rip and/or tear such that the leaflet opening 52 is not a bounded hole. Stated differently, in such examples, the leaflet opening 52 may be formed by a tear that extends from the pilot puncture 50 fully to the free edge of the host leaflet 10 (the coaptation edge of the leaflet).

While a hole-dilating balloon 322 is described above and illustrated for expanding a pilot puncture 50 to form a leaflet opening 52, it is to be understood that other types of expansion member 320 can be used instead of a balloon in any of the methods and/or systems described herein. For example, U.S. Provisional Application No. 63/335,739, which is incorporated herein by reference in its entirety, describes an expandable frame that can be used as an expansion member 320 instead of a valve-expanding balloon.

In some examples, retraction of expansion member 320 such as a hole-dilation balloon 322, after deflation thereof, can be performed while the guidewire 80 may be kept in position, extending through the leaflet opening 52 with the tip 82 positioned distal to the host leaflet 10. Subsequent to recompressing the expansion member 320 inside the leaflet opening 52 and retracting it away from the host leaflet 10, the method can further include steps of positioning a guest prosthetic valve 100 inside the leaflet opening 52. A delivery apparatus carrying the guest prosthetic valve 100 can be either part of the system 200, or provided as a separate assembly advanced into a leaflet opening 52.

FIG. 18I shows a guest prosthetic valve 100 positioned, in a radially compressed configuration thereof, inside the leaflet opening 52. As shown in FIG. 18I, the guest prosthetic valve 100 can be mounted on a replacement valve delivery apparatus 390 that can be advanced towards the host leaflet 10 over a guidewire, which can be a separate guide wire (not shown), or can be the same guidewire 80.

In some examples, the guest prosthetic valve is a balloon expandable valve, and the replacement valve delivery apparatus 390 comprises a balloon catheter 392 carrying a valve-expanding balloon 394. The hole-dilating balloon 322 described above for expanding a leaflet opening 52 is different from a typical valve-expanding balloon 394 used for expanding balloon-expandable prosthetic valves or stents, in that while a typical valve-expanding balloon is inflatable to a diameter that can allow expansion of a prosthetic valve 100 to a functional diameter thereof, which can be similar to, or greater than (for example, in the case of valve over-expansion) the diameter of the native annulus in which the valve is deployed, the maximum diameter of a hole-dilating balloon 322 can be significantly smaller, configured to increase the size of a pilot puncture 50 to form a larger leaflet opening 52, optionally without tearing the host leaflet 10 (though in some examples, the host leaflet 10 may be still torn by a hole-dilating balloon 322).

In some examples, the maximum diameter to which the hole-dilating balloon 322 can be inflated is equal to or less than 12 mm. In some examples, the maximum diameter to which the hole-dilating balloon 322 can be inflated is equal to or less than 10 mm. In contrast, the maximum diameter to which a valve-expanding balloon 394 can be inflated can be, in some examples, greater than 16 mm., greater than 18 mm., greater than 20 mm., and/or greater than 24 mm.

While a replacement valve delivery apparatus 390 equipped with a valve-expanding balloon 394 at a distal end portion of a balloon catheter 392 is illustrated, it is to be understood that this is shown by way of illustration and not limitation, and that a replacement valve delivery apparatus 390 can include other shafts and/or mechanisms, for example when utilized to advance and expand other types of replacement prosthetic heart valves, such as self-expandable prosthetic heart valves or mechanically expandable prosthetic heart valves.

In some examples, the replacement valve delivery apparatus 390 can further include a nosecone 396 positioned distal to the valve-expanding balloon 394 (or other prosthetic-valve expanding mechanism). The nosecone 396 can be conical or frustoconical in shape. The nosecone 396 can be attached to a distal end of a nosecone shaft 398 extending through the balloon catheter 392, wherein the nosecone 396 and the nosecone shaft 398 can collectively define a lumen through which a guidewire can extend. In some examples, when a nosecone 396 is present at a distal end of the replacement valve delivery apparatus 390 as also shown in the example illustrated in FIG. 18I, the nosecone 396 can be advanced towards the host leaflet 10, and may optionally have a maximal diameter that can be somewhat greater than the diameter of the opening 52, such that as the nosecone 396 is inserted into the leaflet opening 52 it can optionally further expand the leaflet opening 52 to a greater diameter.

As shown in FIG. 18I, the guest prosthetic valve 100 is placed in the leaflet opening 52 in its radially compressed configuration, optionally positioned over a deflated valve-expanding balloon 394 in the case of a balloon-expandable prosthetic valve. With the prosthetic valve 100 received within the leaflet opening 52, radially expanding the guest prosthetic valve 100, as shown in FIG. 18J, can serve to increase a size of the leaflet opening 52 and/or to tear the leaflet. As a result, and as discussed above, radially expanding the guest prosthetic valve 100 can serve to modify the host leaflet 10 such that the leaflet does not obstruct a cell opening 112 in a frame 102 of the guest prosthetic valve 100 or at least increases the area of the host valve and the guest valve that is not covered or obstructed by the modified host leaflet to permit access and sufficient perfusion to the adjacent coronary artery. For example, radially expanding the guest prosthetic valve within the leaflet opening 52 can operate to push a portion of the leaflet extending radially exterior of the guest prosthetic valve below an upper edge of an outer skirt of the guest prosthetic valve 100 and/or away from one or more cell openings 112 of the guest prosthetic valve 100.

In some examples, more than one guidewire can be utilized in a method that includes forming the leaflet opening 52 and/or positioning a guest prosthetic valve 100 therein. For example, a first guidewire 80 can be utilized in a method of forming a leaflet opening 52, after components of the system for forming a leaflet opening 52 can be retracted along with the guidewire 80, and a separate guidewire can be then used for advancing a guest prosthetic valve 100 in the host valvular structure. In some examples, a separate guidewire over which a guest prosthetic valve can be advanced, can extend alongside the guidewire 80 over which components for forming a leaflet opening 52 extend.

In some examples, the guest prosthetic valve can be a mechanically-expandable prosthetic valve and radial expansion thereof can be achieved by actuating a mechanical actuator of the guest prosthetic valve to mechanically expand a frame of the guest prosthetic valve. In some examples, the guest prosthetic valve can be a self-expandable prosthetic valve that can be retained during delivery toward the host valvular structure in a capsule or other restraint disposed therearound, and valve expansion can be achieved by removing the capsule or other restraint from the guest prosthetic valve to allow it to radially self-expand within the host valvular structure.

FIGS. 19A-20B illustrate a sequence of events in which a host valvular structure 12 is modified to receive a guest prosthetic valve 100. FIGS. 19A-19B illustrate the hole-dilating balloon 322 utilized to expand the pilot puncture 50 into the leaflet opening 52. In particular, FIG. 19A illustrates the hole-dilating balloon 322 in a deflated state within the pilot puncture 50, corresponding to the state described above with respect to FIG. 18F, while FIG. 19B illustrates the hole-dilating balloon 322 in an inflated state such that the pilot puncture 50 has enlarged into the leaflet opening 52, corresponding to the state described above with respect to FIG. 18G. FIG. 19C illustrates a guest prosthetic valve 100 that can be positioned in the leaflet opening 52 after removal of the hole-dilating balloon 322 therefrom, in a crimped configuration of the prosthetic valve 100, corresponding to the state described above with respect to FIG. 18H, after which the guest prosthetic valve 100 can be expanded, such as by inflating a valve-expanding balloon 394 over which it can be mounted in the case of a balloon-expandable valve, so as to implant the guest prosthetic valve 100 inside the host valvular structure 12.

As mentioned, any system and method of the current specification can be utilized for forming a leaflet opening 52 in a host leaflet 10 which can be either a native leaflet 30 or a prosthetic valve leaflet 114 of a previously implanted prosthetic valve, such as prosthetic valve 100a of FIG. 3, such as in the case of ViV procedures. FIG. 20A shows a previously implanted prosthetic valve 100a subsequent to forming the leaflet opening 52. FIG. 20B shows a configuration in which a second prosthetic valve 100b has been expanded within the leaflet opening 52 of a host prosthetic valve 100a. In the example of FIG. 20B, the guest prosthetic valve 100b is the same type of valve as the host prosthetic valve 100a. It is to be understood, however, that ViV procedures may be similarly applied to any other suitable valvular structures, such as different prosthetic valves and/or native heart valves. For example, the guest prosthetic valve 100b need not be the same type of valve as the host prosthetic valve 100a.

In the example of FIG. 20A, when the prosthetic valve leaflets 114a of the previously implanted prosthetic valve 100a are pressed against the frame 102a, the leaflet opening 52 provides a partial access into the frame 102a, but the leaflet opening 52 may not be sufficiently large to completely uncover any of the cell openings 112a of the frame 102a.

As shown in FIG. 20B, however, fully expanding the guest prosthetic valve 100b within the leaflet opening 52 further expands and/or tears the leaflet opening 52 such that several cell openings 112a of the frame 102a of the host prosthetic valve 100a and several cell openings 112b of the frame 102b of the guest prosthetic valve 100b are fully uncovered by the leaflets 114a. In some examples, this may result from the frame 102b of the guest prosthetic valve 100b pushing the leaflet 114a comprising the leaflet opening 52 downwardly (toward the inflow ends of the prosthetic valves 100a, 100b) such that one or more cell openings 112a are unobstructed by the leaflet 114a. In some examples, expanding the frame 102b within the leaflet 114a comprising the leaflet opening 52 may rip and/or tear this leaflet 114a such that the leaflet 114a cannot obstruct one or more cell openings 112a.

FIGS. 21A and 21B are cross-sectional views of a distal portion of an exemplary system 200^{g}, shown in an unbent state and a bent state, respectively, of a needle 310 thereof. System 200^{g} is an exemplary implementation of system 200, and thus can include any of the features described for system 200 throughout the current disclosure, except that the system 200^{g}, equipped with a perforating member 310 that can be a bendable pre-shaped needle 310 of the types described above with respect to system 200^{f}, further includes an expansion member 320, such as hole-dilating balloon 322 mounted on balloon catheter 324, extending through the outer shaft lumen 203, between the needle 310 and the outer shaft 202. System 200^{g} can include any exemplary assembly 210 and tube 260 disclosed herein, such as any of the exemplary rotation control assemblies 210^{a}, 210^{b}, 210^{c}, 210^{d} and/or 210^{e}, and any of the exemplary tubes 260^{a}, 260^{b}, 260^{c} or 260^{d}. The system 200^{g} can optionally comprise the dilator 330 attached to dilator shaft 338, and the hole-dilating balloon 322 can be optionally disposed between a distal end of the balloon catheter 324 and the dilator proximal portion 336 according to any of the examples described above for dilator 330, dilator shaft 338, and/or hole-dilating balloon 322.

The needle 310 extends through the dilator lumen 340 as illustrated in FIGS. 21A-21B, and is configured to be axially movable in the distal and proximal direction relative to any of the dilator shaft 338 and/or the balloon catheter 324. The dilator shaft 338 can extend through the balloon catheter lumen 326, and may be sized such that an annular space is formed within balloon catheter lumen 326 between an inner surface of the balloon catheter 324 and an outer surface of the dilator shaft 338 along the length of balloon catheter 324. This annular space is in fluid communication with one or more inflation openings 328 exposed to an internal cavity of the hole-dilating balloon 322, which can be in fluid communication with a fluid source (for example, a syringe or a pump) that can inject inflation fluid (for example, saline) into the hole-dilating balloon 322, so as to inflate the balloon 322, for example during formation of leaflet opening 52. The pressure of the inflation fluid within hole-dilating balloon 322 may provide the force that allows it to dilate a leaflet opening 52. Further, the balloon catheter lumen 326 may be configured to withdraw fluid from the balloon 322 through the inflation opening(s) 328, to deflate the balloon 322.

The system 200^{g} can be utilized in a similar manner to that described with respect to FIGS. 18A-8J, with the exception that the system 200^{g}includes both the needle 310 and the hole-dilating balloon 322, enabling expansion of the pilot puncture 50 to form a pilot opening 52 by the same system 200^{g}, without the need to advance separate balloon catheter with a hole-dilating balloon thereof after retraction of the needle. Moreover, a system 200^{g} can be provided without a separate overtube 342 disposed over the needle 310, as the dilator 330 and dilator shaft 338 can collectively serve a role of an outer covering member in the same manner described for the overtube 342, and the dilator lumen 340 can take the role of the overtube lumen 344. Thus, as soon as the needle 310 is exposed out of the dilator 330, the exposed portion, which is no longer bound by the inner surface of the dilator 330 and/or dilator shaft 338, is free to assume a bent shape in a similar manner to that described with respect to FIG. 18B. Nevertheless, in some examples, system 200^{g} can further include an additional overtube 342 through which the needle 310 can extend, wherein the overtube 342 can be axially movable inside and relative to the dilator lumen 340.

A needle 310 utilized for perforating a host leaflet 10 to form a pilot puncture 50 will usually have a small-diameter, especially when compared to catheters or shafts of delivery assemblies for prosthetic valve implantation. Thus, when such a low-profile needle 310 needs to be controllably rotated to angularly orient a distal portion thereof in a desired direction, it can serve as a thin-walled tube 260 having wall thickness T_{w} small enough to warrant utilization of a rotation control assembly 210 according to any of the examples described herein.

In some examples, a bendable needle 310 extending through a covering member, such as an overtube 342 or a dilator 330, can be axially movable relative to the overtube 342 and/or the dilator 330 but rotationally locked there-against. For example, a needle 310 can include one or more elongated protrusions extending radially outward and accepted in one or more corresponding elongated slots along an inner surface of the overtube 342 and/or dilator shaft 338 with dilator 330, such that the needle can axially slide relative to the overtube 342 and/or dilator shaft 338, but rotational movement of the overtube 342 and/or dilator shaft 338 imparts rotation of the needle 310 therewith. In some examples, the tube 260 is an overtube 342, and the inner shaft 274 extending therethrough can be a needle 310. In some examples, the tube 260 is a dilator shaft 338, and the inner shaft 274 extending therethrough can be a needle 310.

In some cases, a perforating member 310 of a system 200, such as a needle, is not shape-set to bend in a free state thereof, in which case additional bending or steering components or assemblies can be included in the system 200 to facilitate controlled bending of the perforating member 310. FIGS. 22A-22B show and exemplary steerable tube assembly 350 which includes a pull-member 372 with one or more elongated pull-arms 376 disposed between an outer bendable tube 356 and an inner bendable tube 364. FIGS. 23A-23B show a distal portion of an exemplary system 200^{h}, which is an exemplary implementation of system 200, and thus can include any of the features described for system 200 throughout the current disclosure, except that the system 200^{h} further comprises the steerable tube assembly 350 of FIG. 22A-22B, which defines a steerable assembly lumen 352 through which a perforating member 310, such as a needle, extends. FIG. 22A is a perspective view of a distal portion of the exemplary steerable tube assembly 350. FIG. 22B is a perspective view of a distal portion of the steerable tube assembly 350 of FIG. 22A, with the outer bendable tube 356 removed from view for illustrative purpose. FIG. 23A and 23B are cross-sectional side views of a distal portion of the system 200^{h} in unbent and bent states, respectively, of the steerable tube assembly 350. FIGS. 22A-23B are described herein together.

System 200^{h} can include any exemplary assembly 210 and tube 260 disclosed herein, such as any of the exemplary rotation control assemblies 210^{a}, 210^{b}, 210^{c}, 210^{d} and/or 210^{e}, and any of the exemplary tubes 260^{a}, 260^{b}, 260^{c} or 260^{d}. The steerable tube assembly 350 can be axially movable through, and relative to, the outer shaft 202, which can be either a non-steerable or a steerable outer shaft 202. The outer bendable tube 356 of the steerable tube assembly 350 extends proximally from an outer tube distal end portion 358 towards the handle 204, and includes an outer tube slotted portion 360 along at least part of its length, such as along at least a distal section of the tube 356 adjacent outer tube distal end portion 358.

In some examples, the outer tube slotted portion 360 is formed as a hypotube, configured to increase flexibility of the outer bendable tube 356 along the outer tube slotted portion 360. The outer tube slotted portion 360 can include a plurality of slits 362 arranged in a manner that can provide sufficient flexibility to allow it to flex, either as it is pushed through a tortuous pathway or when the tube assembly 350 is actively articulated by actuating the pull-member 372, without kinking or buckling.

The inner bendable tube 364 of the steerable tube assembly 350 extends proximally from an inner tube distal end portion 366 towards the handle 204, and includes an inner tube slotted portion 368 along at least part of its length, such as along at least a distal section of the tube 364 adjacent inner tube distal end portion 366. In some examples, the inner tube slotted portion 368 is formed as a hypotube, configured to increase flexibility of the inner bendable tube 364 along the inner tube slotted portion 368. The inner tube slotted portion 368 can include a plurality of slits 370 arranged in a manner that can provide sufficient flexibility to allow it to flex, either as it is pushed through a tortuous pathway or when the tube assembly 350 is actively articulated by actuating the pull-member 372, without kinking or buckling.

The pull-member 372 comprises a pull-ring portion 374 and at least one elongated pull-arm 376 extending proximally from the pull-ring portion 374 towards and into the handle 204. In some examples, the at least one elongated pull-arm 376 is attached to the pull-ring portion 374. In some examples, the at least one elongated pull-arm 376 is integrally formed with the pull-ring portion 374, together forming a unitary pull-member 372. In some examples, the pull-member 372 is a tube-cut pull-member, which can be formed by cutting (for example, laser cutting) a tubular member to form one or more elongated pull-arms 376.

The outer tube distal end portion 358 can be a portion of the outer bendable tube 356 which is devoid of slits 362, and the inner tube distal end portion 366 can be a portion of the inner bendable tube 364 which is devoid of slits 370. The pull-ring portion 374 is affixed, directly or indirectly, both to the outer tube distal end portion 358 and the inner tube distal end portion 366, such as by welding, soldering, gluing, and the like. In contrast, any elongated pull-arm 376 of the pull-member 372 is disposed between an outer surface of the inner bendable tube 364 and an inner surface of the outer bendable tube 356 without being attached thereto, such that the elongated pull-arm 376 can axially slide within the space defined between the tubes 356 and 364 relative to the outer bendable tube 356 and/or the inner bendable tube 364.

In some examples, the distal portion of the steerable tube assembly 350 can include a distal tip portion 354 distal to the pull-ring portion 374 of the pull-member 372. The distal tip portion 354 can be configured to be relatively rigid and not sufficiently flexible to bend, to provide structural support to a component extendable through and out of the steerable tube assembly 350, such as a perforating member 310. The distal tip portion 354 can include a polymeric layer distal to the pull-ring portion 374 that is devoid of the outer tube slotted portion 360 and/or the inner tube slotted portion 368. The distal tip portion 354 can terminate at a distal atraumatic end, which can be optionally rounded and/or curved radially inwards, or can be otherwise formed to include an outer surface tapering in the distal direction.

The curvature of the steerable tube assembly 350, at least along a distal portion thereof, can be changed based on the operator manipulating the elongated pull-arm 376 via an actuator of the handle 204. The elongated pull-arm 376 can extend into the handle 204 and be coupled to a mechanism (not shown) controlled by a handle actuator, such as a knob 206 or any other type of actuator, that can be utilized to axially pull the elongated pull-arm 376 in a proximal direction.

When a proximally-oriented force is applied to the elongated pull-arm 376, the force is transmitted, via the pull-ring portion 374, to the outer tube distal end portion 358 and the inner tube distal end portion 366, resulting in circumferential slits 362 and 370 being narrowed or closed at the regions circumferentially aligned with the position of the axially pulled elongated pull-arm 376, bending the distal portion of the steerable tube assembly 350 in the direction of closure. When the proximally-oriented pull force is released, the distal portion of the steerable tube assembly 350 resumes its straightened (or less bent) configuration.

Resilience of the material of any of the outer bendable tube 356 and the inner bendable tube 364 can be configured to assist the steerable tube assembly 350 in returning to a straighter (or less bent) condition, when the elongated pull-arm 376 is released (i.e., no longer proximally pulled, or proximally pulled at a smaller pulling force). The shape of the slits and the material from which each of the tubes is made, such as Nitinol in some examples, can facilitate "spring-back" of the outer bendable tube 356 and/or inner bendable tube 364 to the pre-bent configuration. This can be advantageous because the elongated pull-arm 376 can be made of a relatively rigid material, such as metal (though other suitable materials are contemplated) that will not be compressed, thus avoiding kinks.

In contrast to a pull-wire utilized in conventional steerable shafts, having a circular cross-section, the elongated pull-arm 376 has a an arcuate flattened configuration, defining a thickness in the radial direction which is smaller than a diameter of a circular pull-wire configured to withstand similar pull-forces, which advantageously enables utilization of the steerable tube assembly 350 in low-profile implementations, such as for bending a small-dimensioned needle 310 passable through the steerable assembly lumen 352.

A pull-member 372 can include any number of elongated pull-arms 376. In some examples, the pull-member 372 includes four elongated pull-arms 376 that can be circumferentially disposed at 90° from each other. In such an arrangement, the steerable tube assembly 350 can be controllably steered in two planes orthogonal to each other. In some examples, the pull-member 372 includes two elongated pull-arms 376 that can be circumferentially disposed at 180° from each other. In such an arrangement, the steerable tube assembly 350 can be controllably steered in two directions across a plane defined by the two elongated pull-arms 376. In some examples, the pull-member 372 includes a single elongated pull-arm 376 extending proximally from the pull-ring portion 374. In such an arrangement, the steerable tube assembly 350 can be bent in a single direction defined by the circumferential position of the elongated pull-arm 376.

A steerable tube assembly 350 disclosed herein can be optionally torqued in a desired rotational direction. Thus, even when a single pull-arm 376 is provided to provide bending in a single predefined direction, or two pull-arms 376 are provided across each other to enable bending in a single plane, rotation of the steerable tube assembly 350, such as by a rotation control assembly 210 engaged with at least one tube thereof, can angularly orient the distal end of the steerable tube assembly 350, as well as any component extending therefrom, such as a needle 310, in a desired orientation. In some examples, the tube 260 is the outer bendable tube 356. In some examples, the tube 260 in the inner bendable tube 364. In some examples, the thin-walled tube 260 comprises both the outer bendable tube 356 and inner bendable tube 364.

In some examples, the steerable tube assembly 350 and the perforating member 310 are configured to be movable axially relative to each other in the proximal and distal directions. In some examples, the perforating member distal end portion 314 is not necessarily configured to be axially translatable relative to the steerable tube assembly 350, in which case it is positioned distal to the distal tip portion 354 at all times.

As shown in FIGS. 23A-23B, a steerable tube assembly 350 can extend through the outer shaft lumen 203. In some examples, the steerable tube assembly 350 is axially movable through, and relative to, the outer shaft 202. As shown in FIG. 23B, when the steerable tube assembly 350 is bent, for example by proximally pulling a corresponding elongated pull-arm 376 thereof, the portion of the perforating member, such as needle 310, extending through the articulating portion of the steerable tube assembly 350, is bent therewith. Thus, a steerable tube assembly 350 can be utilized to actively orient the perforating member 310 in a desired direction.

FIGS. 24A-24C illustrate some steps in a method for utilizing a system 200^{h} for forming an opening within a target tissue. An exemplary implementation of the method is illustrated in FIGS. 24A-24C with respect to forming a leaflet hole inside a host leaflet, which can be performed prior to implanting a guest prosthetic valve inside the host valvular structure. The system 200^{h} can be used to perforate a host leaflet 10, such as a native leaflet 30 or a prosthetic valve leaflet 114 of a previously implanted prosthetic valve.

The distal end portion of the system 200^{h} is configured to be inserted into a patient’s vasculature, such as within an ascending aorta, and to be advanced towards the host leaflet 10. Positioning the system 200^{h} relative to the host leaflet 10 may comprise advancing the system 200^{h} toward the leaflet over a guidewire 80. As mentioned, the needle lumen 312 can be configured to accommodate a guidewire 80 that can be passed therethrough. In such examples, the guidewire 80 can be inserted into the patient’s vasculature, and then the needle 310, along with steerable tube assembly 350 and/or other shafts or tubes of the system 200^{h}, may be advanced toward the host leaflet 10 over the guidewire 80.

During delivery, the needle distal end portion 314 can be retained inside the steerable assembly lumen 352 and/or within the outer shaft lumen 203, such that the sharp needle tip 318 is concealed inside the corresponding lumen, as illustrated in FIG. 17A. This position conceals the needle tip 318 from the surrounding anatomy, to protect the anatomical structures from being engaged or punctured by the needle tip 318 during advancement towards the site of treatment.

When the distal end portion of system 200^{h} lands at the target site, it may be initially positioned at a position that is different than the position of the desired host leaflet 10. If the outer shaft 202 is a steerable shaft, it can be bent to navigate the distal portion of the system 200^{h} toward the desired host leaflet 10, such as a leaflet that can be closer to the left coronary ostium. In some cases, orienting the system 200^{h}sideways, towards a host leaflet 10, optionally in some proximity to the nadir of the leaflet, can orient the distal end of the system 200^{h} towards a host interior surface 14, which can be the interior surface of the aortic wall if the host valve is the native valve, or an interior surface of a frame of a previously implanted prosthetic valve serving as the host valve. In such cases, merely advancing a perforating member such as needle 310 in the distal direction to expose it prior to penetrating through the host leaflet 10, can direct the needle towards the host interior surface 14 instead of the host leaflet 10 itself.

In order to properly orient the needle 310, a distal portion of the steerable tube assembly 350 can be exposed out of the outer shaft 202 and bent, by pulling an appropriate elongated pull-arm 376 thereof as described above, advantageously orienting the distal tip portion 354 toward the host leaflet 10, as illustrated in FIG. 24B, such that during advancement of the needle 310, its distal end portion 314 can contact and pierce through the host leaflet 10, without posing a risk of contacting and damaging adjacent anatomical structures, such as the host interior surface 14. Exposing the steerable tube assembly 350 out of the outer shaft 202 can be accomplished by distally pushing the steerable tube assembly 350 relative to the outer shaft 202, by proximally pulling the outer shaft 202 relative to the steerable tube assembly 350, or both.

As shown in FIG. 24C, the needle 310 is configured to puncture the host leaflet 10 to form a pilot puncture 50 within host leaflet 10, for example when its distal end portion 314 is axially translated relative to the distal tip portion 354 of steerable catheter assembly 210. The guidewire 80 can be optionally advanced through the needle lumen 312 to terminate with guidewire tip 82 distal to the pilot puncture 50 of host leaflet 10 as also shown in FIG. 24C.

Subsequent to forming the pilot puncture 50 and optionally advancing the guidewire 80 to extend therethrough, the needle 310 can be optionally retracted, along with the steerable tube assembly 350. In some examples, the guidewire 80 can be advanced to terminate distal to the host leaflet 10 after formation of the pilot puncture 50 by the needle 310, as illustrated in FIG. 24C. In some examples, the guidewire 80 can be advanced simultaneously with advancement of the needle 310 towards the host leaflet 10 and/or during formation of the pilot puncture 50. In some examples, the needle 310 can be pulled back into the steerable assembly lumen 352 while the steerable tube assembly 350 may remain in position. In some examples, the steerable tube assembly 350 can be retracted simultaneously with, or subsequent to, retraction of needle 310.

After retraction of the needle 310 and steerable tube assembly 350 from the host leaflet 10, an expansion member 320, such as a hole-dilating balloon 322 that can be optionally carried by a balloon catheter 324, can be advanced, optionally along with dilator 330 and dilator shaft 338, towards the pilot puncture, and utilized to expand the pilot puncture 50 to form a leaflet opening 52 according to any of the examples described above with respect to FIGS. 18D-18H. Similarly, after formation of a leaflet opening 52, a replacement valve delivery apparatus 390 can be used to position a guest prosthetic valve 100 inside the host valvular structure 12, and to expand the guest prosthetic valve 100, optionally by inflating a valve0expanding balloon 394 mounted on a balloon catheter 392 in the case of a balloon-expandable prosthetic valve 100, according to any of the examples described above with respect to FIGS. 18I-18J. Further examples of steerable tube assemblies and methods for utilization thereof can be found, for example, in U.S. Provisional Application No. 63/583,915, which is incorporated herein by reference in its entirety.

As mentioned in various examples throughout the current disclosure, a system 200 can include a perforating member 310 for piercing a leaflet 10. The leaflet is a relatively thin tissue having a free edge opposite to attachment on an opposite end to the aortic wall in the case of a native leaflet of an aortic valve, or to a frame of a previously implanted prosthetic valve in the case of ViV procedures. Perforation of the leaflet by pushing a needle or other perforating tool there-against, can move the leaflet to some extent due to the push force applied thereto. Even if the needle or type of perforation member 310 does puncture eventually through the tissue material, such initial movement can lead to the penetration point being in a different region of the leaflet relative to the initial point of contact. Thus, it may be desired, in some examples, to include a mechanism configured to stabilize the leaflet during penetration of a perforating member 310, such as a needle, therethrough.

FIG. 25 shows an exemplary system 200ⁱ. FIGS. 26A and 26B show a perspective sectional view and a cross-sectional side view of a distal portion of the system 200ⁱ of FIG. 25. System 200ⁱ is an exemplary implementation of system 200, and thus can include any of the features described for system 200 throughout the current disclosure, except that the system 200^{g} further comprises a helical anchor 384 at a distal end of an anchor shaft 380. System 200^{g} can include any exemplary assembly 210 and tube 260 disclosed herein, such as any of the exemplary rotation control assemblies 210^{a}, 210^{b}, 210^{c}, 210^{d} and/or 210^{e}, and any of the exemplary tubes 260^{a}, 260^{b}, 260^{c} or 260^{d}. The term "helical anchor 384" and "anchor 384", as used herein, are interchangeable.

The anchor 384 defines an anchor channel 386 and has an anchor sharp tip 388 configured to allow it to engage and penetrate a host leaflet 10 of a host valvular structure. The anchor 384 can be optionally used in combination with a perforating member 310 such as a needle that can extend through the anchor channel 386 towards and through the host leaflet 10, for modifying the host leaflet 10. The anchor shaft 380 can extend through the outer shaft lumen 203. The outer shaft 202 and the anchor shaft 380 can be configured to be axially movable relative to each other. For example, a distally oriented movement of the anchor shaft 380 relative to the outer shaft 202, can expose the anchor 284 from the outer shaft lumen 203.

The anchor shaft 380 can be a torque shaft, configured to be movable rotatably relative to a central axis thereof and/or rotatable relative to another shaft of the system 200, such as relative to the outer shaft 202. The anchor 384 is affixed to the anchor shaft 380 such that rotation of the anchor shaft 380 effects rotation of the anchor 384 therewith. The anchor shaft 380 defines an anchor shaft lumen 382 which is continuous with the anchor channel 386. In some examples, at least a portion of the anchor shaft 380 is formed as a hypotube, configured to increase flexibility thereof. In some examples, the tube 260 is the anchor shaft 380, and the inner shaft 274 can be an optional perforating member 310 extending through the anchor shaft lumen 382, such as a needle 310. The proximal portion of the anchor shaft 380 extends into the handle 204 and is engaged by the rotation control assembly 210, such that rotation of the assembly 210 in a desired direction will rotate the anchor shaft 380 and the helical anchor 384 therewith in the same direction.

In some examples, a helical anchor 384 can be a tube-cut anchor. Manufacturing of a tube-cut helical anchor 384 can employ any suitable cutting method, such as, but not limited to, laser cutting, water-jet cutting, plasma cutting, and the like. In some examples, the helical anchor can be formed from a rounded wire shaped to form turns of the anchor 384. Sharpening the anchor tip 388 can employ grinding or any other suitable sharpening method. In some examples, the helical anchor 384 can include at least one helical slot extending distally from a proximal end of the anchor 384, and defining one or more helical turns continuously extending between the anchor's proximal end and the anchor tip 388. It is to be understood that a reference to a helical anchor 384 attached to an anchor shaft 380 can refer either to a helical anchor 384 provided as a separate component coupled to a distal end of an anchor shaft 380, or to a unitary component wherein the helical anchor 384 is integrally formed with the anchor shaft 380.

The helical anchor 384 can be used with or without a separate perforating member, such as a needle 310 axially movable relative to the helical anchor 384, to form a pilot puncture 50 in a target tissue, such as a host leaflet 10. In some examples, as illustrated in FIGS. 25-26B, the system 200ⁱ further includes a perforating member 310, which can be optionally implemented as a needle 310 extendable through the anchor shaft lumen 382 and anchor channel 386. The needle 310 can be axially movable relative to the helical anchor 384. As further illustrated in FIGS. 25-26A, the helical anchor 384 and anchor shaft 380 can be axially extendable through a lumen 203 of outer shaft 202.

FIGS. 27A-27C illustrate some steps in a method for utilizing the system 200ⁱ of FIGS. 25-26A for forming an opening within a target tissue. An exemplary implementation of the method is illustrated in FIGS. 27A-27C with respect to forming a leaflet hole inside a host leaflet, which can be performed prior to implanting a guest prosthetic valve inside the host valvular structure. The system 200ⁱcan be used to perforate a host leaflet 10, such as a native leaflet 30 or a prosthetic valve leaflet 114 of a previously implanted prosthetic valve.

The distal end portion of the system 200ⁱ is configured to be inserted into a patient’s vasculature, such as within an ascending aorta, and to be advanced towards the host leaflet 10. Positioning the distal end portion the outer shaft 202 and/or the helical anchor 384 relative to the host leaflet 10 may comprise advancing the outer shaft 202 and/or anchor shaft 380 toward the leaflet over a guidewire 80. When a system 200ⁱ includes an additional perforating member axially movable through anchor channel 386, such as a hollow needle 310, the needle lumen 312 can be configured to accommodate the guidewire 80 extendable through the needle lumen 312. In such examples, the guidewire 80 can be inserted into the patient’s vasculature, and then the hollow needle 310 and/or other shafts or tubes of the system 200ⁱ may be advanced toward the host leaflet 10 over the guidewire 80.

When a system 200ⁱ includes a helical anchor 384 configured to form a pilot puncture 50 without the use of an additional perforating member 310, or when the perforating member 310 is a guidewire 80, as will be described in further detail below, the anchor shaft lumen 382 and anchor channel 386 can be configured to accommodate the guidewire 80 extendable through the anchor shaft lumen 382 and anchor channel 386. In such examples, the guidewire 80 can be similarly inserted into the patient’s vasculature, and then the anchor shaft 380 and/or other shafts of the system 200ⁱ may be advanced toward the host leaflet 10 over the guidewire 80.

As mentioned above, a helical anchor 384, along with the anchor shaft 380 attached thereto, is configured to be selectively translated in the proximal or distal directions relative to another component of the system 200ⁱ, such as outer shaft 202, as well as being rotatable around the central longitudinal axis C_{A} by the rotation control assembly 210. Thus, in additional to the rotation control assembly 210, the handle 204 can include a mechanism (not shown) for controlling axial movement of the anchor shaft 380 serving as the tube 260. In some examples, the anchor shaft 380 and the needle 310 are configured to be movable axially relative to each other in the proximal and distal directions.

During delivery, the helical anchor 384 can be retained inside outer shaft lumen 203, such that the anchor sharp tip 388 is at or proximal to a distal end of the outer shaft 202, as illustrated in FIG. 27A. This position conceals the sharp tip 388 of the helical anchor 384 from the surrounding anatomy, to protect the anatomical structures from being engaged or punctured by the anchor sharp tip 388 during advancement towards the site of treatment. When used in combination with a needle 310, the needle distal end portion 314 can be retained inside the anchor channel 386 or anchor shaft lumen 382, such that the needle tip 318 is at or proximal to the anchor tip 388, which is also concealed inside the outer shaft lumen 203, as illustrated in FIG. 27A. This position conceals the sharp tip 318 of the needle 310 from the surrounding anatomy, to similarly protect the anatomical structures from being engaged or punctured by the sharp needle tip 318 during advancement towards the site of treatment.

The anchor shaft 380 can be axially translated in a distal direction, so as to expose the helical anchor 384 out of the outer shaft 202 and position the anchor tip 388 in closer proximity to the host leaflet 10. The anchor shaft 380 is then rotated by a user of the handle 204 rotating the assembly 210 in an appropriate direction (such as clockwise 92 or counterclockwise 94), rotating the helical anchor 384 therewith, causing it to engage and penetrate the host leaflet 10, thereby securing the helical anchor 384 to host leaflet 10 as shown in FIG. 27B. The tissue material of host leaflet 10 can be retained within the anchor's helical slot, between successive helical turns of the anchor 384. The terms "anchor sharp tip 388" and "anchor tip 388", as used herein, are interchangeable.

While FIG. 27B illustrates the anchor shaft 380 with anchor 384 advanced distally relative to the outer shaft 202 prior to and/or during anchoring to the host leaflet 10, in some examples, the outer shaft 202 can be advanced up to contact with the host leaflet 10 prior to anchoring of the anchor into the leaflet 10. The outer shaft 202 can apply a limited extent of a distally oriented push-force against the host leaflet 10, during extension of the anchor 384 distally and rotatably into the host leaflet 10 to anchor against the host leaflet 10. The outer shaft 202, in such examples, can provide an external support structure through which the anchor 384 can be advanced and anchored into the leaflet 10, in a manner that increases stability of the leaflet for improved engagement with the helical anchor 384 at it is being screwed thereinto.

A perforating member, such as a needle 310, can be then distally advanced to puncture the host leaflet 10 to form a pilot puncture 50 within host leaflet 10 as shown in FIG. 27C, for example when its distal end portion 314 is axially translated relative to anchor shaft 380. An attempt to pass a perforating member such as needle 310 through a relatively thin and movable tissue component, such as a leaflet, in the absence of an anchor, might push the leaflet to some extent prior to eventually penetrating therethrough, which, even if achieving the goal of eventually puncturing the leaflet, might result in a wrong or somewhat offset position of the puncture hole due to this undesired relative movement. Advantageously, the helical anchor 384 of systems 200ⁱ described herein, captures the host leaflet 10 and stabilizes it during formation of a pilot puncture 50, such as by a needle 310 being pushed against and through the host leaflet 10.

Once the needle distal end portion 314 is positioned, at least partially, past the host leaflet 10, the guidewire 80 can be advanced through the needle lumen 312 to terminate with guidewire tip 82 at a position distal to the pilot puncture 50 of host leaflet 10 as also shown in FIG. 27C.

Subsequent to forming the pilot puncture 50 and optionally advancing the guidewire 80 to extend therethrough, the needle 310 can be optionally retracted, and the anchor shaft can be rotated in a counter rotational direction, opposite to the rotational direction employed for anchoring it to the leaflet 10, so as to release the helical anchor 384 from the host leaflet 10, which can be similarly retracted by being then axially pulled away from the host leaflet 10, optionally while leaving the guidewire 80 extending through the pilot puncture 50.

It is to be understood that the order of procedural steps described above can be modified, and that reverse rotation of the helical anchor 384 to release it from the host leaflet 10 and retract it can be performed prior to needle 310 retraction. In some examples, counter-rotation of the helical anchor 384 to release it from the host leaflet 10 can be performed prior to needle 310 retraction, and axial retraction of the helical anchor 384 can be performed subsequent to needle 310 retraction. In some examples, needle 310 retraction can be performed simultaneously with counter-rotation of the helical anchor 384 to release it from the host leaflet 10 and/or axial retraction of the helical anchor 384 from the host leaflet.

In some examples, the guidewire 80 can be advanced simultaneously with advancement of the needle 310 during formation of the pilot puncture 50. In some examples, the guidewire 80 can be advanced to terminate distal to the host leaflet 10 after formation of the pilot puncture 50 by the needle 310, as illustrated in FIG. 27C. In some examples, the guidewire 80 can be advanced through pilot puncture 50 to terminate distal to the host leaflet 10 after retrieval of the needle 310, optionally prior to release of the anchor 384 from the host leaflet 10.

In some examples, advancement of the guidewire 80 to position the guidewire tip 82 distal to the pilot puncture 50 can be performed subsequent to counter-rotation of the helical anchor 384 to release it from the host leaflet 10 and/or axial retraction of the helical anchor 384, while the needle 310 is still positioned inside of pilot puncture 50, after which the needle 310 can be retracted. In some examples, advancement of the guidewire 80 to position the guidewire tip 82 distal to the pilot puncture 50 can be performed after needle 310 retraction while the helical anchor 384 is still engaged with the host leaflet 10, after which the anchor 384 can be release and retracted.

After retraction of the needle 310 and the anchor 384 from the host leaflet 10, an expansion member 320, such as a hole-dilating balloon 322 that can be optionally carried by a balloon catheter 324, can be advanced, optionally along with dilator 330 and dilator shaft 338, towards the pilot puncture, and utilized to expand the pilot puncture 50 to form a leaflet opening 52 according to any of the examples described above with respect to FIGS. 18D-18H. Similarly, after formation of a leaflet opening 52, a replacement valve delivery apparatus 390 can be used to position a guest prosthetic valve 100 inside the host valvular structure 12, and to expand the guest prosthetic valve 100, optionally by inflating a valve-expanding balloon 394 mounted on a balloon catheter 392 in the case of a balloon-expandable prosthetic valve 100, according to any of the examples described above with respect to FIGS. 18I-18J.

As mentioned above, a helical anchor 384 can be designed, in some examples, to form a pilot puncture 50 and/or leaflet opening 52 without a separate perforating member. In some examples, the width of the helical turn defined by the anchor 384 can increase in the proximal direction from the anchor tup 388. In such cases, as the anchor sharp tip 388 initially penetrates into the tissue, it forms an initial point of entry, which is expanded in size to a larger cut portion as the anchor 384 is rotated through the tissue, due to the increase in the width of the helical turns passing through the tissue, wherein the rate of increase and size of the width can be designed to cut through a portion of a circumference of the tissue, sufficient to form a pilot puncture 50 or opening 52, which can be generally sized according to the diameter of anchor channel 386. Thus, a helical anchor 384 having a turn width gradually increasing in the proximal direction from the anchor tip 388, can be utilized for forming a pilot puncture 50 or opening 52, even in the absence of a separate perforation member, such as a needle 310.

In some examples, any helical anchor 384 configured to serve as a perforating member 310 by cutting around a circumference of an eventually formed pilot puncture 50 or opening 52, is utilized to form a circumferential cut that does not extend along the entire circumference of the final opening 50 or 52. Rotational movement of the helical anchor 384, controlled by the extent of rotation of the assembly 210, can be limited to form a cut that spans more than 180° but less than 360° around the central axis C_{A}. Forming a cut spanning 360° may cause the cut circular portion of the tissue to "fall-off" and be carried by the blood stream, posing a risk of forming clots that can result in adverse clinical outcomes. Forming a cut that spans less than 180° can result in a circumferential cut that allows only a limited portion of the tissue to be movable and allow extension of other components, such as dilator 330 and/or expansion member 320, to extend therethrough.

In some examples, the helical anchor 384 can include a sharp cutting edge at a proximal end of the anchor's helical slot, configured to cut through the tissue during rotation of the anchor 384 to extend through the tissue material. Further examples of further examples of systems equipped with a helical anchor, and methods for utilization thereof, can be found, for example, in U.S. Provisional Application No. 63/540,757, which is incorporated herein by reference in its entirety.

In some examples, the guidewire 80 of any system 200 or method described herein, can be used as a perforating or lacerating member for forming a pilot puncture 50. A perforating guidewire 80 can be used instead of, or in addition to, a needle and/or a perforating helical anchor 384. In such examples, the guidewire 80 can be a relatively stiff wire having a distal tip 82 configured to pierce the host leaflet 10 when the guidewire 80 is pressed against the leaflet. In some examples, the guidewire 80 can include a radio-frequency (RF) energy delivery tip 82 to assist with penetration through the leaflet tissue. For this purpose, a suitable RF energy device may be coupled to the guidewire 80, and the RF energy device can apply the RF energy to the guidewire tip 82 to penetrate the host leaflet 10.

In any example disclosed herein wherein a guidewire is used to puncture a leaflet, the guidewire can be coupled to a source of RF energy that applies RF energy to the tip of the guidewire. When the guidewire 80 is used to pierce the leaflet 10, a needle can be omitted, or it can be used in combination with the guidewire 80 that forms an initial puncture in the leaflet 10. For example, the guidewire 80 can be used to form an initial pilot puncture 50, after which a needle 310 can be advanced through the leaflet to form a slightly larger pilot puncture for subsequent advancement of the dilator 330 through the host leaflet 10.

In some examples, the guidewire 80 is used as a perforating member 310 without any additional separate perforating member, such as a needle or a perforating helical anchor, disposed thereover, such that the guidewire 80 can be utilized as the sole component that forms the pilot puncture 50.

In some examples, the guidewire 80 is used as a perforating member that can be used in addition to another perforating member, such as a needle, such that the guidewire 80 can form an initial puncture via a sharp tip 82 or an RF energy delivery tip 82, followed by penetration of needle 310 into the leaflet 10 to form the pilot puncture 50, or a pilot puncture 50 which is greater in size than an initial puncture formed by the guidewire tip 82.

In some examples, the guidewire tip 82 is not necessarily sharp enough or otherwise configured to puncture through the host leaflet 10, in which case the guidewire 80 can be utilized for advancement of the system 200 and/or shafts thereof toward the valvular structure 12, but terminate in proximity of the host leaflet 10 without piercing through it, and in the case of a system 200ⁱ, a helical anchor 384 can be then rotated and advanced to engage with and pass through the host leaflet 10, and can be optionally configured to form the pilot puncture 50, or be used in combination with a needle 310 advanced into the stabilized tissue to form the pilot puncture 50.

In some examples, a system 200 that includes a rotation control assembly 210 is part of a delivery assembly that further includes the guest prosthetic valve 100 carried, in a radially compressed state thereof, over a component of the system 200. Exemplary delivery assemblies that include perforating members that can be implemented in the form of a needle, a first balloon that can be a hole-dilating balloon 322, and a second balloon that can be valve-expanding balloon 394, are described in U.S. Provisional Application Nos. 63/447,453 and 63/447,457, each of which is incorporated herein by reference in its entirety.

Any of the systems, devices, assemblies, etc. herein can be sterilized (for example, with heat, radiation, and/or chemicals, etc.) to ensure they are safe for use with patients, and any of the methods herein can include sterilization of the associated assembly, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

### Some Examples of the Disclosed Technology

Some examples of above-described technology are enumerated below. It should be noted that one feature of an example in isolation or more than one feature of the example taken in combination and, optionally, in combination with one or more features of one or more examples below are examples also falling within the disclosure of this application.

Example 1. A system comprising:

a handle comprising a rotation control assembly which comprises:

a rotatable body defining an aperture;

at least one first protrusion comprising a first contact surface and configured to extend radially into the aperture;

at least one second protrusion comprising a second contact surface and configured to extend radially into the aperture;

a tube comprising a tube wall having a tube wall thickness and defining a tube lumen, wherein the tube extends through the aperture and comprises:

at least one first window radially extending through the tube wall and defining a first contact edge; and

at least one second window radially extending through the tube wall and defining a second contact edge.

Example 2. The system of any example herein, particularly of example 1, wherein the at least one first protrusion extends into the at least one first window, and the at least one second protrusion extends into the at least one second window.

Example 3. The system of any example herein, particularly of example 1 or 2, wherein the first contact surface and the second contact surface face circumferentially opposite directions.

Example 4. The system of any example herein, particularly of any one of examples 1 to 3, wherein, when the rotatable body is rotated in a first circumferential direction, the first contact surface is pushed against the first contact edge, causing the tube to rotate in the first circumferential direction along with the rotatable body.

Example 5. The system of any example herein, particularly of example 4, wherein, when the rotatable body is rotated in a second circumferential direction which is opposite to the first circumferential direction, the second contact surface is pushed against the second contact edge, causing the tube to rotate in the second circumferential direction along with the rotatable body.

Example 6. The system of any example herein, particularly of any one of examples 1 to 5, wherein the rotation control assembly comprises at least one first lug embedded in the rotatable body and at least one second lug embedded in the rotatable body, wherein the at least one first protrusion is defined as a portion of the corresponding first lug protruding radial from the rotatable body into the aperture, and wherein the at least one second protrusion is defined as a portion of the corresponding second lug protruding radially from the rotatable body into the aperture.

Example 8. The system of any example herein, particularly of examples 6 or 7, wherein the first circumferential direction is a clockwise direction, and wherein the second circumferential direction is a counterclockwise direction.

Example 7. The system of any example herein, particularly of example 6, wherein the at least one first lug resides inside at least one first lug bore of the rotatable body, and the at least one second lug resides inside at least one second lug bore of the rotatable body.

Example 9. The system of any example herein, particularly of example 8, wherein the at least one first lug comprises a clockwise facing sidewall and a counterclockwise facing sidewall, wherein the at least one second lug comprises a counterclockwise facing sidewall and a clockwise facing sidewall, wherein the first contact surface is defined as a portion of the clockwise facing sidewall of the at least one first lug that extends into the aperture, and wherein the second contact surface is defined as a portion of the counterclockwise facing sidewall of the at least one second lug that extends into the aperture.

Example 10. The system of any example herein, particularly of example 9, wherein the counterclockwise facing sidewall of the at least one first lug does not extend into the aperture, and wherein the clockwise facing sidewall of the at least one second lug does not extend into the aperture.

Example 11. The system of any example herein, particularly of example 9 or 10, wherein the counterclockwise facing sidewall of the at least one first lug does not extend into the corresponding first window, and wherein the clockwise facing sidewall of the at least one second lug does not extend into the corresponding second window.

Example 12. The system of any example herein, particularly of any one of examples 6 to 11, wherein the at least one first lug and the at least one second lug are stiffer than the rotatable body.

Example 13. The system of any example herein, particularly of example 12, wherein the at least one first lug and the at least one second lug are made of a metallic material.

Example 14. The system of any example herein, particularly of example 12 or 13, wherein the rotatable body is made of a polymeric material.

Example 15. The system of any example herein, particularly of any one of examples 12 to 14, wherein the rotatable body is overmolded over the at least one first lug and the at least one second lug.

Example 16. The system of any example herein, particularly of any one of examples 1 to 15, wherein the at least one first protrusion and the at least one second protrusion do not protrude into the tube lumen.

Example 17. The system of any example herein, particularly of any one of examples 1 to 16, wherein the tube wall thickness is not greater to 200µ.

Example 18. The system of any example herein, particularly of example 17, wherein the tube wall thickness is between 50µ and 200µ.

Example 19. The system of any example herein, particularly of any one of examples 1 to 18, wherein the tube wall thickness is less than 15% of an outer diameter of the tube.

Example 20. The system of any example herein, particularly of example 19, wherein the tube wall thickness is less than 10% of the outer diameter of the tube.

Example 21. The system of any example herein, particularly of example 19, wherein the tube wall thickness is less than 8% of the outer diameter of the tube.

Example 22. The system of any example herein, particularly of any one of examples 1 to 21, wherein the tube wall thickness is less than 15% of a diameter of the aperture.

Example 23. The system of any example herein, particularly of example 19, wherein the tube wall thickness is less than 10% of the diameter of the aperture.

Example 24. The system of any example herein, particularly of example 19, wherein the tube wall thickness is less than 8% of the diameter of the aperture.

Example 25. The system of any example herein, particularly of any one of examples 1 to 24, wherein a maximal contact surface radial length defined by any of the first contact surface and the second contact surface is configured to prevent extension of any of the at least one first protrusion and any of the at least one second protrusion into the tube lumen.

Example 26. The system of any example herein, particularly of example 24, wherein the maximal contact surface radial length is not greater than about 300µ.

Example 27. The system of any example herein, particularly of example 24, wherein the maximal contact surface radial length is not greater than about 200µ.

Example 28. The system of any example herein, particularly of any one of examples 25 to 27, wherein the maximal contact surface radial length is not greater than 20% of a diameter of the aperture.

Example 29. The system of any example herein, particularly of any one of examples 25 to 28, wherein the maximal contact surface radial length is not greater than 15% of the diameter of the aperture.

Example 30. The system of any example herein, particularly of any one of examples 25 to 29, wherein the maximal contact surface radial length is not greater than 10% of the diameter of the aperture.

Example 31. The system of any example herein, particularly of any one of examples 1 to 30, wherein the rotatable body comprises at least two body portions releasably attached to each other.

Example 32. The system of any example herein, particularly of any one of examples 1 to 31, wherein the at least one first protrusion and the at least one second protrusion are integrally formed with the rotatable body.

Example 33. The system of any example herein, particularly of any one of examples 1 to 32, wherein the at least one first protrusion comprises two first protrusions, and the at least one second protrusion comprises two second protrusions.

Example 34. The system of any example herein, particularly of example 33, wherein the two first protrusions are circumferentially offset from each other, and wherein the two second protrusions are circumferential offset from each other.

Example 35. The system of any example herein, particularly of example 34, wherein the two first protrusions extend from radially opposing sides of an inner surface defined by the rotatable body, and wherein the two second protrusions extend from radially opposing sides of the inner surface.

Example 36. The system of any example herein, particularly of example 34 or 35, wherein the two first protrusions are axially aligned with each other, and wherein the two second protrusions are axially aligned with each other.

Example 37. The system of any example herein, particularly of any one of examples 33 to 36, wherein the first contact surface of each of the two first protrusions is circumferentially aligned with the second contact surface of the corresponding second radial protrusion of the two second protrusions.

Example 38. The system of any example herein, particularly of any one of examples 33 to 37, wherein the two first protrusions are axially offset from the two second protrusions.

Example 39. The system of any example herein, particularly of any one of examples 1 to 32, wherein the at least one first protrusion comprises one first protrusion, and the at least one second protrusion comprises one second protrusion.

Example 40. The system of any example herein, particularly of example 39, wherein the one first protrusion is axially offset from the one second protrusion.

Example 41. The system of any example herein, particularly of example 40, wherein the first contact surface of the one first protrusion is circumferentially aligned with the second contact surface of the one second protrusion.

Example 42. The system of any example herein, particularly of example 40, wherein the one first protrusion is circumferentially offset from the one second protrusion.

Example 43. The system of any example herein, particularly of example 42, wherein the one first protrusion and the one second protrusion extend from radially opposing sides of an inner surface defined by the rotatable body.

Example 44. The system of any example herein, particularly of example 39, wherein the one first protrusion is axially aligned with the one second protrusion.

Example 45. The system of any example herein, particularly of example 44, wherein the one first protrusion is circumferentially offset from the one second protrusion.

Example 46. The system of any example herein, particularly of example 45, wherein the one first protrusion and the one second protrusion extend from radially opposing sides of an inner surface defined by the rotatable body.

Example 47. The system of any example herein, particularly of any one of examples 1 to 46, further comprising an inner shaft extending through the tube lumen, wherein none of the at least one first protrusion and the at least one second protrusion extend into the inner shaft.

Example 48. The system of any example herein, particularly of example 47, wherein the inner shaft is axially movable relative to the tube.

Example 49. The system of any example herein, particularly of any one of examples 1 to 48, further comprising a perforating member extending distally from the handle, the perforating member configured to pierce a target tissue to form a pilot puncture in the target tissue.

Example 50. The system of any example herein, particularly of example 49, wherein the perforating member defines a perforating member lumen.

Example 51. The system of any example herein, particularly of example 50, wherein the perforating member comprises a needle, and wherein the perforating member lumen comprises a needle lumen.

Example 52. The system of any example herein, particularly of example 51, wherein the needle comprises a needle distal end portion defining an angled surface.

Example 53. The system of any example herein, particularly of example 52, wherein the needle distal end portion terminates at a needle tip.

Example 54. The system of any example herein, particularly of any one of examples 51 to 53, wherein the needle is biased to a bent state in a free state thereof.

Example 55. The system of any example herein, particularly of example 54, wherein the needle is made of a shape-memory material.

Example 56. The system of any example herein, particularly of example 55, wherein the shape-memory material comprises Nitinol.

Example 57. The system of any example herein, particularly of any one of examples 51 to 53, further comprising an overtube defining an overtube lumen, wherein the needle extends through the overtube lumen.

Example 58. The system of any example herein, particularly of example 57, wherein the needle is axially movable relative to the overtube.

Example 59. The system of any example herein, particularly of any one of examples 49 to 58, wherein the perforating member is the tube.

Example 60. The system of any example herein, particularly of example 57 or 58, wherein the overtube is the tube.

Example 61. The system of any example herein, particularly of any one of examples 49 to 56, further comprising a dilator attached to a dilator shaft extending proximally therefrom, wherein the perforating member extends through a dilator lumen collectively defined by the dilator and the dilator shaft.

Example 62. The system of any example herein, particularly of example 61, wherein the dilator comprises a dilator tapering portion.

Example 63. The system of any example herein, particularly of example 61 or 62, wherein the perforating member is axially movable relative to the dilator.

Example 64. The system of any example herein, particularly of any one of examples 61 to 63, wherein the dilator shaft is the tube.

Example 65. The system of any example herein, particularly of any one of examples 49 to 56, further comprising a steerable tube assembly defining s steerable assembly lumen through which the perforating member extends, wherein the steerable tube assembly comprises:

an inner bendable tube comprising an inner tube slotted portion and an inner tube distal end portion distal to the inner tube slotted portion;

an outer bendable tube disposed around the bendable inner tube, the outer bendable tube comprising an outer tube slotted portion and an outer tube distal end portion distal to the inner tube slotted portion; and

a pull-member comprising a pull-ring portion affixed to the inner tube distal end portion and to the outer tube distal end portion, and at least one elongated pull-arm extending proximally from the pull-ring portion;

wherein the at least one elongated pull-arm is disposed between and is axially slidable relative to the inner tube and the outer tube; and

wherein the at least one elongated pull-arm is configured to bend the steerable tube assembly when the at least one elongated pull-arm is proximally pulled.

Example 66. The system of any example herein, particularly of example 65, wherein the tube comprises at least one of the outer bendable tube and the inner bendable tube.

Example 67. The system of any example herein, particularly of any one of examples 1 to 53, further comprising an anchor shaft defining an anchor shaft lumen, and a helical anchor attached to the anchor shaft, the helical anchor defining an anchor channel continuous with the anchor shaft lumen, wherein the anchor shaft is a flexible torque shaft configured to rotate around a central axis thereof, such that when the anchor shaft is rotated, the helical anchor is configured to rotate therewith.

Example 68. The system of any example herein, particularly of example 67, wherein the anchor comprises an anchor tip configured to penetrate through a target tissue during rotational movement of the helical anchor.

Example 69. The system of any example herein, particularly of example 67 or 68, wherein the anchor shaft is the tube.

Example 70. A method comprising:

advancing a system comprising a tube, over a guidewire, to a target tissue, the system comprising a handle comprising a rotation control assembly, wherein the rotation control assembly comprises a rotatable body defining an aperture through which the tube extends, a first protrusion extending radially from the rotatable body into a first window of the tube, and a second protrusion extending radially from the rotatable body into a second window of the tube; and

rotating the rotatable body in a first circumferential direction or a second circumferential direction;

wherein rotating the rotatable body in the first circumferential direction causes a first contact surface thereof to push against a first contact edge of the first window, thereby causing the tube to rotate in the first circumferential direction along with the rotatable body;

wherein rotating the rotatable body in the second circumferential direction causes a second contact surface thereof to push against a second contact edge of the second window, thereby causing the tube to rotate in the second circumferential direction along with the rotatable body; and

wherein the first contact surface and the second contact surface face circumferentially opposite directions.

Example 71. The method of any example herein, particularly of example 70, wherein the first protrusion and the second protrusion do not protrude into a tube lumen defined by the tube.

Example 72. The method of any example herein, particularly of example 70 or 71, wherein the tube comprises a tube wall having a wall thickness that is not greater to 200µ.

Example 73. The method of any example herein, particularly of any one of examples 70 to 72, wherein the rotation control assembly comprises a first lug embedded in the rotatable body and a second lug embedded in the rotatable body, wherein the first protrusion is defined as a portion of the first lug protruding radial from the rotatable body into the first window, and wherein the second protrusion is defined as a portion of the second lug protruding radially from the rotatable body into the second window.

Example 74. The method of any example herein, particularly of example 73, wherein the first lug and the second lug are stiffer than the rotatable body.

Example 75. The method of any example herein, particularly of any one of examples 70 to 74, further comprising forming, with a perforating member tip of a perforating member of the system extending distally from the handle, a pilot puncture within a target tissue.

Example 76. The method of any example herein, particularly of example 75, wherein the system further comprises an outer shaft defining an outer shaft lumen, and wherein the perforating member is disposed inside the outer shaft lumen during the advancing of the target tissue.

Example 77. The method of any example herein, particularly of example 76, wherein the advancing the system to target tissue comprises retaining the perforating member tip inside the outer shaft lumen.

Example 78. The method of any example herein, particularly of example 77, further comprising, after the advancing the system and before the forming the pilot puncture, advancing the perforating member so as to expose the perforating member tip out of the outer shaft lumen.

Example 79. The method of any example herein, particularly of any one of examples 75 to 78, wherein the perforating member defines a perforating member lumen through which the guidewire extends.

Example 80. The method of any example herein, particularly of example 79, wherein the forming the pilot puncture comprises perforating the target tissue by the guidewire, followed by piercing the target tissue by the perforating member, advanced over the guidewire to form the pilot puncture.

Example 81. The method of any example herein, particularly of example 80, wherein the perforating the target tissue by the guidewire comprises applying RF energy to a tip of the guidewire.

Example 82. The method of any example herein, particularly of any one of examples 75 to 81, wherein the perforating member comprises a needle, wherein the perforating member lumen comprises a needle lumen, and wherein the perforating member tip comprises a needle tip.

Example 83. The method of any example herein, particularly of example 82, wherein the needle comprises a needle distal end portion defining an angled surface.

Example 84. The method of any example herein, particularly of example 82 or 83, further comprising, prior to the forming the pilot puncture, transitioning the needle to an uncovered bent state thereof.

Example 85. The method of any example herein, particularly of example 84, wherein the needle is configured to bias towards the uncovered bent state in a free state of the needle.

Example 86. The method of any example herein, particularly of example 85, wherein the needle is made of a shape-memory material.

Example 87. The method of any example herein, particularly of example 86, wherein the shape-memory material comprises Nitinol.

Example 88. The method of any example herein, particularly of any one of examples 84 to 87, wherein the system further comprises an overtube defining an overtube lumen through which the needle extends.

Example 89. The method of any example herein, particularly of example 88, wherein the transitioning the needle to the uncovered bent state comprises advancing the needle to expose a portion thereof out of the overtube.

Example 90. The method of any example herein, particularly of any one of examples 84 to 89, wherein the needle is the tube.

Example 91. The method of any example herein, particularly of example 88 or 89, wherein the needle is axially movable relative to the overtube but is rotationally locked against the overtube, such that rotational movement of the overtube imparts rotation of the needle.

Example 92. The method of any example herein, particularly of example 91, wherein the overtube is the tube.

Example 93. The method of any example herein, particularly of any one of examples 84 to 88, wherein the needle extends through a dilator lumen collectively defined by a dilator and a dilator shaft extending proximally therefrom.

Example 94. The method of any example herein, particularly of example 93, further comprising, subsequent to the forming the pilot puncture, passing the dilator through the pilot puncture, thereby further expanding the pilot puncture.

Example 95. The method of any example herein, particularly of example 93 or 94, wherein the transitioning the needle to the uncovered bent state comprises advancing the needle to expose a portion thereof out of the dilator.

Example 96. The method of any example herein, particularly of any one of examples 93 to 95, wherein the needle is axially movable relative to the dilator and the dilator shaft but is rotationally locked against tat least one of the dilator and the dilator shaft, such that rotational movement of the dilator shaft imparts rotation of the needle.

Example 97. The method of any example herein, particularly of example 96, wherein the dilator shaft is the tube.

Example 98. The method of any example herein, particularly of any one of examples 90, 92 or 97, wherein the rotating the rotatable body comprises orienting the needle tip, in the uncovered bent state of the needle, towards the target tissue.

Example 99. The method of any example herein, particularly of any one of examples 75 to 83, wherein the system further comprises a steerable tube assembly defining a steerable assembly lumen through which the perforating member extends, wherein the steerable tube assembly comprises an inner bendable tube, an outer bendable tube disposed around the inner bendable tube, and a pull-member that comprises a pull-ring portion affixed to an inner tube distal end portion of the inner bendable tube and to an outer tube distal end portion of the outer bendable tube, and an elongated pull-arm extending proximally from the pull-ring portion and which is slidingly movable between and relative to the inner bendable tube and the outer bendable tube.

Example 100. The method of any example herein, particularly of example 99, further comprising, prior to the forming the pilot puncture, bending a distal portion of the steerable tube assembly by proximally pulling the elongated pull-arm.

Example 101. The method of any example herein, particularly of example 100, wherein the perforating member is axially movable relative to the steerable tube assembly.

Example 102. The method of any example herein, particularly of example 101, wherein the advancing the system to the target tissue comprises retaining the perforating member tip inside the steerable assembly lumen.

Example 103. The method of any example herein, particularly of example 100 or 101, wherein the forming the pilot puncture comprises advancing the perforating member so as to expose the perforating member tip out of the steerable assembly lumen.

Example 104. The method of any example herein, particularly of any one of examples 100 to 103, wherein the tube comprises at least one of the outer bendable tube and the inner bendable tube.

Example 105. The method of any example herein, particularly of example 104, wherein the rotating the rotatable body comprises orienting a distal tip portion of the steerable tube assembly towards the target tissue.

Example 106. The method of any example herein, particularly of any one of examples 75 to 83, wherein the system further comprises a helical anchor at a distal end of an anchor shaft, the anchor shaft defining an anchor shaft lumen through which the perforation member extends.

Example 107. The method of any example herein, particularly of example 106, wherein the anchor shaft is the tube.

Example 108. The method of any example herein, particularly of example 107, wherein the rotating the rotatable body causes the anchor shaft and the helical anchor to rotate therewith.

Example 109. The method of any example herein, particularly of example 108, wherein the rotating the rotatable body comprises, prior to the forming the pilot puncture, securing the helical anchor to the target tissue.

Example 110. The method of any example herein, particularly of example 109, wherein the securing the helical anchor comprises penetrating the target tissue by an anchor tip of the helical anchor.

Example 111. The method of any example herein, particularly of example 108 or 109, wherein the forming the pilot puncture comprises extending the perforating member through an anchor channel defined by the helical anchor.

Example 112. The method of any example herein, particularly of any one of examples 75 to 111, further comprising, after the forming the pilot puncture, positioning an expansion member inside the pilot puncture, in a compacted state of the expansion member.

Example 113. The method of any example herein, particularly of example 112, further comprising, subsequent to the positioning the expansion member inside the pilot puncture, expanding the expansion member to expand the pilot puncture and form a tissue opening within the target tissue.

Example 114. The method of any example herein, particularly of example 113, wherein the expansion member comprises a hole-dilating balloon mounted on a balloon catheter, wherein the compacted state of the expansion member is a deflated state of the hole-dilating balloon, and wherein the expanding the expansion member comprises inflating the hole-dilating balloon.

Example 115. The method of any example herein, particularly of example 113 or 114, further comprising, subsequent to the expanding the expansion member, transitioning the expansion member back to its compacted state.

Example 116. The method of any example herein, particularly of example 115, wherein the target tissue is a host leaflet of a host valvular structure, and wherein the tissue opening is a leaflet opening.

Example 117. The method claim 116, further comprising, subsequent to the forming the leaflet opening, positioning a guest prosthetic valve in a radially compressed state thereof within the host valvular structure, and radially expanding the guest prosthetic valve.

Example 118. The method of any example herein, particularly of example 117, wherein the positioning the guest prosthetic valve within the host valvular structure comprises positioning the guest prosthetic valve within the leaflet opening.

Example 119. The method of any example herein, particularly of example 117, wherein the positioning the guest prosthetic valve within the host valvular structure comprises positioning the guest prosthetic valve between host leaflets of the host valvular structure.

Example 120. The method of any example herein, particularly of any one of examples 117 to 119, wherein the radially expanding the guest prosthetic valve comprises inflating a valve-expanding balloon over which the guest prosthetic valve is disposed.

Example 121. The method of any example herein, particularly of any one of examples 117 to 119, wherein the radially expanding the guest prosthetic valve comprises actuating a mechanical actuator of the guest prosthetic valve.

Example 122. The method of any example herein, particularly of any one of examples 117 to 119, wherein the guest prosthetic valve is a self-expandable prosthetic valve, and wherein radially expanding the guest prosthetic valve comprises removing a restraint from around the guest prosthetic valve.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A system comprising:
a handle comprising a rotation control assembly which comprises:
a rotatable body defining an aperture;
at least one first protrusion comprising a first contact surface and configured to extend radially into the aperture;
at least one second protrusion comprising a second contact surface and configured to extend radially into the aperture;
a tube comprising a tube wall having a tube wall thickness and defining a tube lumen, wherein the tube extends through the aperture and comprises:
at least one first window radially extending through the tube wall and defining a first contact edge; and
at least one second window radially extending through the tube wall and defining a second contact edge.

2. The system of claim 1, wherein the at least one first protrusion extends into the at least one first window, and the at least one second protrusion extends into the at least one second window.

3. The system of claim 1, wherein the first contact surface and the second contact surface face circumferentially opposite directions.

4. The system of claim 1, wherein, when the rotatable body is rotated in a first circumferential direction, the first contact surface is pushed against the first contact edge, causing the tube to rotate in the first circumferential direction along with the rotatable body.

5. The system of claim 1, wherein, when the rotatable body is rotated in a second circumferential direction which is opposite to the first circumferential direction, the second contact surface is pushed against the second contact edge, causing the tube to rotate in the second circumferential direction along with the rotatable body.

6. The system of claim 1, wherein the rotation control assembly comprises at least one first lug embedded in the rotatable body and at least one second lug embedded in the rotatable body, wherein the at least one first protrusion is defined as a portion of the corresponding first lug protruding radial from the rotatable body into the aperture, and wherein the at least one second protrusion is defined as a portion of the corresponding second lug protruding radially from the rotatable body into the aperture.

7. The system of claim 1, wherein the at least one first protrusion and the at least one second protrusion do not protrude into the tube lumen.

8. The system of claim 1, wherein the tube wall thickness is not greater to 200µ.

9. The system of claim 1, wherein the at least one first protrusion comprises two first protrusions, and the at least one second protrusion comprises two second protrusions.

10. The system of claim 9, wherein the first contact surface of each of the two first protrusions is circumferentially aligned with the second contact surface of the corresponding second radial protrusion of the two second protrusions.

11. The system of claim 9, wherein the two first protrusions are axially offset from the two second protrusions.

12. The system of claim 1, wherein the at least one first protrusion comprises one first protrusion, and the at least one second protrusion comprises one second protrusion.

13. The system of claim 12, wherein the one first protrusion is axially offset from the one second protrusion.

14. The system of claim 13, wherein the first contact surface of the one first protrusion is circumferentially aligned with the second contact surface of the one second protrusion.

15. The system of claim 13, wherein the one first protrusion is circumferentially offset from the one second protrusion.

16. The system of claim 12, wherein the one first protrusion is axially aligned with the one second protrusion.

17. A method comprising:
advancing a system comprising a tube, over a guidewire, to a target tissue, the system comprising a handle comprising a rotation control assembly, wherein the rotation control assembly comprises a rotatable body defining an aperture through which the tube extends, a first protrusion extending radially from the rotatable body into a first window of the tube, and a second protrusion extending radially from the rotatable body into a second window of the tube; and
rotating the rotatable body in a first circumferential direction or a second circumferential direction;
wherein rotating the rotatable body in the first circumferential direction causes a first contact surface thereof to push against a first contact edge of the first window, thereby causing the tube to rotate in the first circumferential direction along with the rotatable body;
wherein rotating the rotatable body in the second circumferential direction causes a second contact surface thereof to push against a second contact edge of the second window, thereby causing the tube to rotate in the second circumferential direction along with the rotatable body; and
wherein the first contact surface and the second contact surface face circumferentially opposite directions.

18. The method of claim 17, further comprising forming, with a perforating member tip of a perforating member of the system extending distally from the handle, a pilot puncture within a target tissue.

19. The method of claim 18, wherein the perforating member comprises a needle defining a needle lumen through which the guidewire extends, and wherein the perforating member tip comprises a needle tip.

20. The method of claim 19, wherein the needle is the tube.

21. The method of claim 18, wherein the system further comprises a helical anchor at a distal end of an anchor shaft, the anchor shaft defining an anchor shaft lumen through which the perforation member extends.

22. The method of claim 21, wherein the anchor shaft is the tube.

23. The method of claim 22, wherein the rotating the rotatable body causes the anchor shaft and the helical anchor to rotate therewith.

24. The method of claim 23, wherein the rotating the rotatable body comprises, prior to the forming the pilot puncture, securing the helical anchor to the target tissue.
